SEQUENCE LISTING (187 sequences)

SEQ ID NO: 1 - protein, 197 aa, Homo sapiens
QVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVA

SEQ ID NO: 2 - protein, 97 aa, Homo sapiens
VNCSQFLRGQECVEECRVLQGLPREYVNARHCLPCHPECQPQNGSVTCFGPEADQCVACAHYKDPPFCVARCPSGVKPDLSYMPIWKFPDEEGACQP

SEQ ID NO: 3 - protein, 302 aa, Artificial Sequence
  a designed akyrin repeat protein domain binding the extracellular domain 1 of HER2 and a designed akyrin repeat protein domain binding the extracellular domain 4 of HER2 connected by a glycine/serine peptide linker and with a N-terminal His-tag
MRGSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGETAEDLAKDNGNQDIADLLEKALKLGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 4 - protein, 335 aa, Artificial Sequence
  a designed akyrin repeat protein domain binding the extracellular domain 1 of HER2 and a designed akyrin repeat protein domain binding the extracellular domain 4 of HER2 connected by a glycine/serine peptide linker and with a N-terminal His-tag
MRGSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGETAEDLAKDNGNQDIADLLEKALKLGGGGSRSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLN

SEQ ID NO: 5 - protein, 298 aa, Artificial Sequence
  a designed akyrin repeat protein domain binding the extracellular domain 1 of HER2 and a designed akyrin repeat protein domain binding the extracellular domain 4 of HER2 connected by a glycine/serine peptide linker and with a N-terminal His-tag
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 6 - protein, 331 aa, Artificial Sequence
  a designed akyrin repeat protein domain binding the extracellular domain 1 of HER2 and a designed akyrin repeat protein domain binding the extracellular domain 4 of HER2 connected by a glycine/serine peptide linker and with a N-terminal His-tag
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSRSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLN

SEQ ID NO: 7 - protein, 336 aa, Artificial Sequence
  a designed akyrin repeat protein domain binding the extracellular domain 1 of HER2 and a designed akyrin repeat protein domain binding the extracellular domain 4 of HER2 connected by a glycine/serine peptide linker and with a N-terminal His-tag
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSGGGGSRSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLN

SEQ ID NO: 8 - protein, 341 aa, Artificial Sequence
  a designed akyrin repeat protein domain binding the extracellular domain 1 of HER2 and a designed akyrin repeat protein domain binding the extracellular domain 4 of HER2 connected by a glycine/serine peptide linker and with a N-terminal His-tag
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSGGGGSGGGGSRSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLN

SEQ ID NO: 9 - protein, 346 aa, Artificial Sequence
  a designed akyrin repeat protein domain binding the extracellular domain 1 of HER2 and a designed akyrin repeat protein domain binding the extracellular domain 4 of HER2 connected by a glycine/serine peptide linker and with a N-terminal His-tag
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSGGGGSGGGGSGGGGSRSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLN

SEQ ID NO: 10 - protein, 158 aa, Artificial Sequence
  designed akyrin repeat protein ligand binding the extracellular domain 1 of the HER2
GSDLGKKLLEAARAGQDDEVRILMANGADVNASDYYGITPLHLAAHTGHLEIVEVLLKHGADVNARNWGWTPLHLAAMTGHLEIVEVLLKYGADVNAVDEDGDTPLHLAATHGHLEIVEVLLKYGADVNAQDKFGKTAFDISIDNGNEDLAEILQKLN

SEQ ID NO: 11 - protein, 160 aa, Artificial Sequence
  designed akyrin repeat protein ligand binding the extracellular domain 1 of the HER2
GSDLGKKLLEAARAGQDDEVRILMANGADVNAKDWYGITPLHLAADTGHLEIVEVLLKHGADVNAFDSYTGHTPLHLAAQKGQLEIVEVLLKYGADVNAIDRHGKTPLHLAALMGHLEIVEVLLKYGADVNAQDKFGKTAFDISIDNGNEDLAEILQKLN

SEQ ID NO: 12 - protein, 125 aa, Artificial Sequence
  designed akyrin repeat protein ligand binding the extracellular domain 1 of the HER2
GSDLGKKLLEAARAGQDDEVRILMANGADVNASDFYGKTPLHLAATIGHLKIVEVLLKYGADVNATDWGNTPLHLAAINGHLEIVEVLLKHGADVNAQDKFGKTAFDISIDNGNEDLAEILQKLN

SEQ ID NO: 13 - protein, 159 aa, Artificial Sequence
  designed akyrin repeat protein ligand binding the extracellular domain 1 of the HER2
GSDLGKKLLEAARAGQDDEVRILMANGADVNAHDFHGLTPLHLAAGMGHLEIVEVLLKNGADVNAVDTDGITLLHLAAYYGHLEIVEVLLKHGADVNAHDYAGSTPLHLAANTGHLEIVEVLLKNGADVNAQDKFGKTAFDISIDNGNEDLAEILQKLN

SEQ ID NO: 14 - protein, 159 aa, Artificial Sequence
  designed akyrin repeat protein ligand binding the extracellular domain 1 of the HER2
GSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTAFDISIDNGNEDLAEILQKLN

SEQ ID NO: 15 - protein, 159 aa, Artificial Sequence
  designed akyrin repeat protein ligand binding the extracellular domain 1 of the HER2
GSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTPFDLAIDNGNEDIAEVLQKAA

SEQ ID NO: 16 - protein, 159 aa, Artificial Sequence
  designed akyrin repeat protein ligand binding the extracellular domain 1 of the HER2
GSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTAEDLAKDNGNQDIADLLEKAL

SEQ ID NO: 17 - protein, 159 aa, Artificial Sequence
  designed akyrin repeat protein ligand binding the extracellular domain 1 of the HER2
GSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTAMDLARDNGNEDIYKLLAKAL

SEQ ID NO: 18 - protein, 158 aa, Artificial Sequence
  designed akyrin repeat protein ligand binding the extracellular domain 1 of the HER2
GSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLN

SEQ ID NO: 19 - protein, 158 aa, Artificial Sequence
  designed akyrin repeat protein ligand binding the extracellular domain 1 of the HER2
GSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAA

SEQ ID NO: 20 - protein, 158 aa, Artificial Sequence
  designed akyrin repeat protein ligand binding the extracellular domain 1 of the HER2
GSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAEDLAKDNGNQDIADLLEKAL

SEQ ID NO: 21 - protein, 158 aa, Artificial Sequence
  designed akyrin repeat protein ligand binding the extracellular domain 1 of the HER2
GSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAMDLARDNGNEDIYKLLAKAL

SEQ ID NO: 22 - protein, 159 aa, Artificial Sequence
  designed akyrin repeat protein ligand binding the extracellular domain 1 of the HER2
GSDLGKKLLETARAGQDDEVRILMANGADVNATDFYGLTPLHLAAYYGHLEIVEVLLKNGADVNASDWNGYTPLRLAADAGHLEIVEVLLKNGADVNAFDQFGSTPLHLAAATGHLEIVEVLLKYGADVNAQDKFGKTAFDISIDNGNEDLAEILQKLN

SEQ ID NO: 23 - protein, 158 aa, Artificial Sequence
  designed akyrin repeat protein ligand binding the extracellular domain 1 of the HER2
GSDLGKKLLEAARAGQDDEVRILMANGADVNAHDFYGKTPLHLAAAIGHLEIVEVLLKNGADVNATDYGLTPLHLAADNGHLEIVEVLLKNGADVNAFDFTGRTPLHLAASQGHLEIVEVLLKNGADVNAQDKFGKTAFDISIDNGNEDLAEILQKLN

SEQ ID NO: 24 - protein, 160 aa, Artificial Sequence
  designed akyrin repeat protein ligand binding the extracellular domain 1 of the HER2
GSDLGKKLLEAARAGQDDEVRILMANGADVNAHDWHGITPLHLAAFYGHLEIVEVLLKNGADVNAFDDYDGSTPLHLAAWMGHLEIVEVLLKHGADVNATDHFGNTPLHLAAAMGHLEIVEVLLKHGADVNAQDKFGKTAFDISIDNGNEDLAEILQKLN

SEQ ID NO: 25 - protein, 126 aa, Artificial Sequence
  designed akyrin repeat protein ligand binding the extracellular domain 4 of the HER2
GSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 26 - protein, 159 aa, Artificial Sequence
  designed akyrin repeat protein ligand binding the extracellular domain 4 of the HER2
GSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLN

SEQ ID NO: 27 - protein, 159 aa, Artificial Sequence
  designed akyrin repeat protein ligand binding the extracellular domain 4 of the HER2
GSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTPFDLAIDNGNEDIAEVLQKAA

SEQ ID NO: 28 - protein, 159 aa, Artificial Sequence
  designed akyrin repeat protein ligand binding the extracellular domain 4 of the HER2
GSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAEDLAKDNGNQDIADLLEKAL

SEQ ID NO: 29 - protein, 159 aa, Artificial Sequence
  designed akyrin repeat protein ligand binding the extracellular domain 4 of the HER2
GSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAMDLARDNGNEDIYKLLAKAL

SEQ ID NO: 30 - protein, 158 aa, Artificial Sequence
  designed akyrin repeat protein ligand binding the extracellular domain 1 of the HER2
GSDLGKKLLEAARAGQDDEVRILMANGADVNASDYYGITPLHLAAHTGHLEIVEVLLKHGADVNARNWGWTPLHLAAMTGHLEIVEVLLKYGADVNAVDEDGDTPLHLAATHGHLEIVEVLLKYGADVNAQDKFGKTPFDLAIDNGNEDIAEVLQKAA

SEQ ID NO: 31 - protein, 158 aa, Artificial Sequence
  designed akyrin repeat protein ligand binding the extracellular domain 1 of the HER2
GSDLGKKLLEAARAGQDDEVRILMANGADVNASDYYGITPLHLAAHTGHLEIVEVLLKHGADVNARNWGWTPLHLAAMTGHLEIVEVLLKYGADVNAVDEDGDTPLHLAATHGHLEIVEVLLKYGADVNAQDKFGKTAEDLAKDNGNQDIADLLEKAL

SEQ ID NO: 32 - protein, 158 aa, Artificial Sequence
  designed akyrin repeat protein ligand binding the extracellular domain 1 of the HER2
GSDLGKKLLEAARAGQDDEVRILMANGADVNASDYYGITPLHLAAHTGHLEIVEVLLKHGADVNARNWGWTPLHLAAMTGHLEIVEVLLKYGADVNAVDEDGDTPLHLAATHGHLEIVEVLLKYGADVNAQDKFGKTAMDLARDNGNEDIYKLLAKAL

SEQ ID NO: 33 - protein, 160 aa, Artificial Sequence
  designed akyrin repeat protein ligand binding the extracellular domain 1 of the HER2
GSDLGKKLLEAARAGQDDEVRILMANGADVNAKDWYGITPLHLAADTGHLEIVEVLLKHGADVNAFDSYTGHTPLHLAAQKGQLEIVEVLLKYGADVNAIDRHGKTPLHLAALMGHLEIVEVLLKYGADVNAQDKFGKTPFDLAIDNGNEDIAEVLQKAA

SEQ ID NO: 34 - protein, 160 aa, Artificial Sequence
  designed akyrin repeat protein ligand binding the extracellular domain 1 of the HER2
GSDLGKKLLEAARAGQDDEVRILMANGADVNAKDWYGITPLHLAADTGHLEIVEVLLKHGADVNAFDSYTGHTPLHLAAQKGQLEIVEVLLKYGADVNAIDRHGKTPLHLAALMGHLEIVEVLLKYGADVNAQDKFGKTAEDLAKDNGNQDIADLLEKAL

SEQ ID NO: 35 - protein, 160 aa, Artificial Sequence
  designed akyrin repeat protein ligand binding the extracellular domain 1 of the HER2
GSDLGKKLLEAARAGQDDEVRILMANGADVNAKDWYGITPLHLAADTGHLEIVEVLLKHGADVNAFDSYTGHTPLHLAAQKGQLEIVEVLLKYGADVNAIDRHGKTPLHLAALMGHLEIVEVLLKYGADVNAQDKFGKTAMDLARDNGNEDIYKLLAKAL

SEQ ID NO: 36 - protein, 125 aa, Artificial Sequence
  designed akyrin repeat protein ligand binding the extracellular domain 1 of the HER2
GSDLGKKLLEAARAGQDDEVRILMANGADVNASDFYGKTPLHLAATIGHLKIVEVLLKYGADVNATDWGNTPLHLAAINGHLEIVEVLLKHGADVNAQDKFGKTPFDLAIDNGNEDIAEVLQKAA

SEQ ID NO: 37 - protein, 125 aa, Artificial Sequence
  designed akyrin repeat protein ligand binding the extracellular domain 1 of the HER2
GSDLGKKLLEAARAGQDDEVRILMANGADVNASDFYGKTPLHLAATIGHLKIVEVLLKYGADVNATDWGNTPLHLAAINGHLEIVEVLLKHGADVNAQDKFGKTAEDLAKDNGNQDIADLLEKAL

SEQ ID NO: 38 - protein, 125 aa, Artificial Sequence
  designed akyrin repeat protein ligand binding the extracellular domain 1 of the HER2
GSDLGKKLLEAARAGQDDEVRILMANGADVNASDFYGKTPLHLAATIGHLKIVEVLLKYGADVNATDWGNTPLHLAAINGHLEIVEVLLKHGADVNAQDKFGKTAMDLARDNGNEDIYKLLAKAL

SEQ ID NO: 39 - protein, 159 aa, Artificial Sequence
  designed akyrin repeat protein ligand binding the extracellular domain 1 of the HER2
GSDLGKKLLEAARAGQDDEVRILMANGADVNAHDFHGLTPLHLAAGMGHLEIVEVLLKNGADVNAVDTDGITLLHLAAYYGHLEIVEVLLKHGADVNAHDYAGSTPLHLAANTGHLEIVEVLLKNGADVNAQDKFGKTPFDLAIDNGNEDIAEVLQKAA

SEQ ID NO: 40 - protein, 159 aa, Artificial Sequence
  designed akyrin repeat protein ligand binding the extracellular domain 1 of the HER2
GSDLGKKLLEAARAGQDDEVRILMANGADVNAHDFHGLTPLHLAAGMGHLEIVEVLLKNGADVNAVDTDGITLLHLAAYYGHLEIVEVLLKHGADVNAHDYAGSTPLHLAANTGHLEIVEVLLKNGADVNAQDKFGKTAEDLAKDNGNQDIADLLEKAL

SEQ ID NO: 41 - protein, 159 aa, Artificial Sequence
  designed akyrin repeat protein ligand binding the extracellular domain 1 of the HER2
GSDLGKKLLEAARAGQDDEVRILMANGADVNAHDFHGLTPLHLAAGMGHLEIVEVLLKNGADVNAVDTDGITLLHLAAYYGHLEIVEVLLKHGADVNAHDYAGSTPLHLAANTGHLEIVEVLLKNGADVNAQDKFGKTAMDLARDNGNEDIYKLLAKAL

SEQ ID NO: 42 - protein, 159 aa, Artificial Sequence
  designed akyrin repeat protein ligand binding the extracellular domain 1 of the HER2
GSDLGKKLLETARAGQDDEVRILMANGADVNATDFYGLTPLHLAAYYGHLEIVEVLLKNGADVNASDWNGYTPLRLAADAGHLEIVEVLLKNGADVNAFDQFGSTPLHLAAATGHLEIVEVLLKYGADVNAQDKFGKTPFDLAIDNGNEDIAEVLQKAA

SEQ ID NO: 43 - protein, 159 aa, Artificial Sequence
  designed akyrin repeat protein ligand binding the extracellular domain 1 of the HER2
GSDLGKKLLETARAGQDDEVRILMANGADVNATDFYGLTPLHLAAYYGHLEIVEVLLKNGADVNASDWNGYTPLRLAADAGHLEIVEVLLKNGADVNAFDQFGSTPLHLAAATGHLEIVEVLLKYGADVNAQDKFGKTAEDLAKDNGNQDIADLLEKAL

SEQ ID NO: 44 - protein, 159 aa, Artificial Sequence
  designed akyrin repeat protein ligand binding the extracellular domain 1 of the HER2
GSDLGKKLLETARAGQDDEVRILMANGADVNATDFYGLTPLHLAAYYGHLEIVEVLLKNGADVNASDWNGYTPLRLAADAGHLEIVEVLLKNGADVNAFDQFGSTPLHLAAATGHLEIVEVLLKYGADVNAQDKFGKTAMDLARDNGNEDIYKLLAKAL

SEQ ID NO: 45 - protein, 158 aa, Artificial Sequence
  designed akyrin repeat protein ligand binding the extracellular domain 1 of the HER2
GSDLGKKLLEAARAGQDDEVRILMANGADVNAHDFYGKTPLHLAAAIGHLEIVEVLLKNGADVNATDYGLTPLHLAADNGHLEIVEVLLKNGADVNAFDFTGRTPLHLAASQGHLEIVEVLLKNGADVNAQDKFGKTPFDLAIDNGNEDIAEVLQKAA

SEQ ID NO: 46 - protein, 158 aa, Artificial Sequence
  designed akyrin repeat protein ligand binding the extracellular domain 1 of the HER2
GSDLGKKLLEAARAGQDDEVRILMANGADVNAHDFYGKTPLHLAAAIGHLEIVEVLLKNGADVNATDYGLTPLHLAADNGHLEIVEVLLKNGADVNAFDFTGRTPLHLAASQGHLEIVEVLLKNGADVNAQDKFGETKEDLAKDNGNQDIADLLEKAL

SEQ ID NO: 47 - protein, 158 aa, Artificial Sequence
  designed akyrin repeat protein ligand binding the extracellular domain 1 of the HER2
GSDLGKKLLEAARAGQDDEVRILMANGADVNAHDFYGKTPLHLAAAIGHLEIVEVLLKNGADVNATDYGLTPLHLAADNGHLEIVEVLLKNGADVNAFDFTGRTPLHLAASQGHLEIVEVLLKNGADVNAQDKFGETKMDLARDNGNEDIYKLLAKAL

SEQ ID NO: 48 - protein, 160 aa, Artificial Sequence
  designed akyrin repeat protein ligand binding the extracellular domain 1 of the HER2
GSDLGKKLLEAARAGQDDEVRILMANGADVNAHDWHGITPLHLAAFYGHLEIVEVLLKNGADVNAFDDYDGSTPLHLAAWMGHLEIVEVLLKHGADVNATDHFGNTPLHLAAAMGHLEIVEVLLKHGADVNAQDKFGKTPFDLAIDNGNEDIAEVLQKAA

SEQ ID NO: 49 - protein, 160 aa, Artificial Sequence
  designed akyrin repeat protein ligand binding the extracellular domain 1 of the HER2
GSDLGKKLLEAARAGQDDEVRILMANGADVNAHDWHGITPLHLAAFYGHLEIVEVLLKNGADVNAFDDYDGSTPLHLAAWMGHLEIVEVLLKHGADVNATDHFGNTPLHLAAAMGHLEIVEVLLKHGADVNAQDKFGKTAEDLAKDNGNQDIADLLEKAL

SEQ ID NO: 50 - protein, 160 aa, Artificial Sequence
  designed akyrin repeat protein ligand binding the extracellular domain 1 of the HER2
GSDLGKKLLEAARAGQDDEVRILMANGADVNAHDWHGITPLHLAAFYGHLEIVEVLLKNGADVNAFDDYDGSTPLHLAAWMGHLEIVEVLLKHGADVNATDHFGNTPLHLAAAMGHLEIVEVLLKHGADVNAQDKFGKTAMDLARDNGNEDIYKLLAKAL

SEQ ID NO: 51 - protein, 5 aa, Artificial Sequence
  designed peptide linker
GGGGS

SEQ ID NO: 52 - protein, 10 aa, Artificial Sequence
  designed peptide linker
GGGGSGGGGS

SEQ ID NO: 53 - protein, 15 aa, Artificial Sequence
  designed peptide linker
GGGGSGGGGSGGGGS

SEQ ID NO: 54 - protein, 20 aa, Artificial Sequence
  designed peptide linker
GGGGSGGGGSGGGGSGGGGS

SEQ ID NO: 55 - protein, 77 aa, Homo sapiens
EDNYALAVLDNGDPLNNTTPVTGASPGGLRELQLRSLTEILKGGVLIQRNPQLCYQDTILWKDIFHKNNQLALTLID

SEQ ID NO: 56 - protein, 27 aa, Homo sapiens
FLRGQECVEECRVLQGLPREYVNARHC

SEQ ID NO: 57 - protein, 65 aa, Homo sapiens
LPCHPECQPQNGSVTCFGPEADQCVACAHYKDPPFCVARCPSGVKPDLSYMPIWKFPDEEGACQP

SEQ ID NO: 58 - protein, 313 aa, Artificial Sequence
  9_20_G, a designed akyrin repeat protein domain binding the extracellular domain 1 of HER2 and a designed akyrin repeat protein domain binding the extracellular domain 4 of HER2 connected by a glycine/serine peptide linker and with a
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSGGGGSGGGGSGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 59 - protein, 303 aa, Artificial Sequence
  9_10_G, a designed akyrin repeat protein domain binding the extracellular domain 1 of HER2 and a designed akyrin repeat protein domain binding the extracellular domain 4 of HER2 connected by a glycine/serine peptide linker and with a
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 60 - protein, 314 aa, Artificial Sequence
  6_20_G, a designed akyrin repeat protein domain binding the extracellular domain 1 of HER2 and a designed akyrin repeat protein domain binding the extracellular domain 4 of HER2 connected by a glycine/serine peptide linker and with a
MRGSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTAFDISIDNGNEDLAEILQKLGGGGSGGGGSGGGGSGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 61 - protein, 168 aa, Artificial Sequence
  9_29, designed akyrin repeat protein ligand binding the extracellular domain 1 of the HER2 with N-terminal His-tag
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLN

SEQ ID NO: 62 - protein, 168 aa, Artificial Sequence
  9_29_newCcap, designed akyrin repeat protein ligand binding the extracellular domain 1 of the HER2 with N-terminal His-tag
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTPFDLAIDNGNEDIAEVLQKAA

SEQ ID NO: 63 - protein, 169 aa, Artificial Sequence
  9_26, designed akyrin repeat protein ligand binding the extracellular domain 1 of the HER2 with N-terminal His-tag
MRGSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTAFDISIDNGNEDLAEILQKLN

SEQ ID NO: 64 - protein, 169 aa, Artificial Sequence
  9_26_new_Ccap, designed akyrin repeat protein ligand binding the extracellular domain 1 of the HER2 with N-terminal His-tag
MRGSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTPFDLAIDNGNEDIAEVLQKAA

SEQ ID NO: 65 - protein, 254 aa, Artificial Sequence
  scFV-A21HL, a scFV binding the extracellular domain 1 of HER2 with a glycine serine linker connecting heavy and light chain
EVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKR

SEQ ID NO: 66 - protein, 254 aa, Artificial Sequence
  scFV A21LH, a scFV binding the extracellular domain 1 of HER2 with a glycine serine linker connecting heavy and light chain
DIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSS

SEQ ID NO: 67 - protein, 248 aa, Artificial Sequence
  scFV 4D5HL, a scFV binding the extracellular domain 4 of HER2 with a glycine serine linker connecting heavy and light chain
EVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSGGGGSGGGGSGGGGSGGGGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKR

SEQ ID NO: 68 - protein, 248 aa, Artificial Sequence
  scFV 4D5LH, a scFV binding the extracellular domain 4 of HER2 with a glycine serine linker connecting heavy and light chain
DIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSS

SEQ ID NO: 69 - protein, 249 aa, Artificial Sequence
  scFV 4D5LH alternative, a scFV binding the extracellular domain 4 of HER2 with an alternative linker connecting heavy and light chain
DIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRATPSHNSHQVPSAGGPTANSGEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSS

SEQ ID NO: 70 - protein, 412 aa, Artificial Sequence
  A21HL_L4_G3, a bispecific HER2 binding agent comprising a scFV antibody fragment binding domain 1 of HER2 and a DARPin binding domain 4 of HER2 connected by a glycine serine linker
AGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLGGGGSGGGGSGGGGSGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLGSHHHHHH

SEQ ID NO: 71 - protein, 412 aa, Artificial Sequence
  A21LH_L4_G3, a bispecific HER2 binding agent comprising a scFV antibody fragment binding domain 1 of HER2 and a DARPin binding domain 4 of HER2 connected by a glycine serine linker
AGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLGGGGSGGGGSGGGGSGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLGSHHHHHH

SEQ ID NO: 72 - protein, 445 aa, Artificial Sequence
  A21HL_L4_H14, a bispecific HER2 binding agent comprising a scFV antibody fragment binding domain 1 of HER2 and a DARPin binding domain 4 of HER2 connected by a glycine serine linker
AGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLGGGGSGGGGSGGGGSGGGGSRSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLGSHHHHHH

SEQ ID NO: 73 - protein, 438 aa, Artificial Sequence
  H14_L4_A21LH, a bispecific HER2 binding agent comprising a DARPin binding domain 4 of HER2 and a scFV antibody fragment binding domain 1 of HER2connected by a glycine serine linker
AGSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLGGGGSGGGGSGGGGSGGGGSRSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLN

SEQ ID NO: 74 - protein, 438 aa, Artificial Sequence
  H14_L4_A21HL, a bispecific HER2 binding agent comprising a DARPin binding domain 4 of HER2 and a scFV antibody fragment binding domain 1 of HER2connected by a glycine serine linker
AGSDLGKKLLEAARAGQDDEVRILMANGADVNATDIHGHTPLHLAAAMGHLEIVEVLLKNGADVNANDWRGFTPLHLAALNGHLEIVEVLLKNGADVNATDTAGNTPLHLAAWFGHLEIVEVLLKNGADVNAQDKFGKTAFDISIDNGNEDLAEILQKLGGGGSGGGGSGGGGSGGGGSRSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLN

SEQ ID NO: 75 - protein, 405 aa, Artificial Sequence
  G3_L4_A21LH, a bispecific HER2 binding agent comprising a DARPin binding domain 4 of HER2 and a scFV antibody fragment binding domain 1 of HER2connected by a glycine serine linker
AGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLGGGGSGGGGSGGGGSGGGGSRSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLN

SEQ ID NO: 76 - protein, 405 aa, Artificial Sequence
  G3_L4_A21HL, a bispecific HER2 binding agent comprising a DARPin binding domain 4 of HER2 and a scFV antibody fragment binding domain 1 of HER2connected by a glycine serine linker
AGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLGGGGSGGGGSGGGGSGGGGSRSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLN

SEQ ID NO: 77 - protein, 397 aa, Artificial Sequence
  A21HL_L1_G3, a bispecific HER2 binding agent comprising a DARPin binding domain 4 of HER2 and a scFV antibody fragment binding domain 1 of HER2connected by a glycine serine linker
AGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLGGGGSRSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLGSHHHHHH

SEQ ID NO: 78 - protein, 414 aa, Artificial Sequence
  9.29_L1_4D5LH, a bispecific HER2 binding agent comprising a DARPin binding domain 1 of HER2 and a scFV antibody fragment binding domain 4 of HER2connected by a glycine serine linker
AGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSRSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRATPSHNSHQVPSAGGPTANSGEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSS

SEQ ID NO: 79 - protein, 521 aa, Artificial Sequence
  diabody A21H_4D5LH_A21L, comprising scFV A21 binding to domain 1 of HER2 and scFV 4D5 binding to domain 4 of HER2
METDTLLLWVLLLWVPGSTGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLHHHHHH

SEQ ID NO: 80 - protein, 548 aa, Artificial Sequence
  4D5HL-L1-A21HL, a bispecific HER2 binding agent comprising a scFV antibody fragment binding domain I of HER2 and a a scFV antibody fragment binding domain 4 of HER2 connected by a glycine serine linker
MKKIWLALAGLVLAFSASADYKDDIGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSGGGGSGGGGSGGGGSGGGGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRKLGGGGSRSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLGSHHHHHH

SEQ ID NO: 81 - protein, 548 aa, Artificial Sequence
  4D5HL-L4-A21LH, a bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSGGGGSGGGGSGGGGSGGGGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRKLGGGGSRSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLGSHHHHHH

SEQ ID NO: 82 - protein, 548 aa, Artificial Sequence
  4D5LH-L1-A21HL, a bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLGGGGSRSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLGSHHHHHH

SEQ ID NO: 83 - protein, 563 aa, Artificial Sequence
  4D5LH-L4-A21HL, a bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLGGGGSGGGGSGGGGSGGGGSRSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLGSHHHHHH

SEQ ID NO: 84 - protein, 563 aa, Artificial Sequence
  4D5LH-L4-A21LH, a bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLGGGGSGGGGSGGGGSGGGGSRSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLGSHHHHHH

SEQ ID NO: 85 - protein, 563 aa, Artificial Sequence
  A21HL_L4_4D5LH, a bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSGGGGSGGGGSGGGGSGGGGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRKLGGGGSGGGGSGGGGSGGGGSRSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLGSHHHHHH

SEQ ID NO: 86 - protein, 548 aa, Artificial Sequence
  A21LH-L1-4D5LH,  a bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLGGGGSRSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLGSHHHHHH

SEQ ID NO: 87 - protein, 563 aa, Artificial Sequence
  A21LH-L4-4D5LH, a bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLGGGGSGGGGSGGGGSGGGGSRSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLGSHHHHHH

SEQ ID NO: 88 - protein, 467 aa, Artificial Sequence
  926E-L4-4D5HL,  a bispecific HER2 binding agent comprising a DARPin binding domain 1 of HER2 and a scFV antibody fragment binding domain 4 of HER2connected by a glycine serine linker
MKKIWLALAGLVLAFSASADYKDDIGSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGETAEDLAKDNGNQDIADLLEKALKLGGGGSGGGGSGGGGSGGGGSRSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSGGGGSGGGGSGGGGSGGGGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRKLN

SEQ ID NO: 89 - protein, 467 aa, Artificial Sequence
  926E-L4-4D5LH, a bispecific HER2 binding agent comprising a DARPin binding domain 1 of HER2 and a scFV antibody fragment binding domain 4 of HER2connected by a glycine serine linker
MKKIWLALAGLVLAFSASADYKDDIGSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGETAEDLAKDNGNQDIADLLEKALKLGGGGSGGGGSGGGGSGGGGSRSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLN

SEQ ID NO: 90 - protein, 463 aa, Artificial Sequence
  929-L4-4D5HL,  a bispecific HER2 binding agent comprising a DARPin binding domain 1 of HER2 and a scFV antibody fragment binding domain 4 of HER2connected by a glycine serine linker
MKKIWLALAGLVLAFSASADYKDDIGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSGGGGSGGGGSGGGGSRSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSGGGGSGGGGSGGGGSGGGGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRKLN

SEQ ID NO: 91 - protein, 463 aa, Artificial Sequence
  929-L4-4D5LH,  a bispecific HER2 binding agent comprising a DARPin binding domain 1 of HER2 and a scFV antibody fragment binding domain 4 of HER2connected by a glycine serine linker
MKKIWLALAGLVLAFSASADYKDDIGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQKLGGGGSGGGGSGGGGSGGGGSRSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLN

SEQ ID NO: 92 - protein, 281 aa, Artificial Sequence
  scFV 4D5LH alternative, a scFV binding the extracellular domain 4 of HER2 with a glycine serine linker connecting heavy and light chain with C-term His-tag and additional N-term peptide
MKKTAIAIAVALAGFATVAQADIGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLHHHHHH

SEQ ID NO: 93 - protein, 287 aa, Artificial Sequence
  scFV A21LH alternative, scFV 4D5 alternative, a scFV binding the extracellular domain 1 of HER2 with a glycine serine linker connecting heavy and light chain with C-term His-tag and additional N-term peptide
MKKTAIAIAVALAGFATVAQADIGSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLHHHHHH

SEQ ID NO: 94 - protein, 27 aa, Artificial Sequence
  N-terminal peptide for periplasmic expression
  SITE (1)..(19): DsbA-signal sequence
  SITE (20)..(25): FLAG-Tag M1
  SITE (26)..(27): BamHI cloning site
MKKIWLALAGLVLAFSASADYKDDIGS

SEQ ID NO: 95 - protein, 24 aa, Artificial Sequence
  a glycine/serine linker comprising a N-terminal HindIII cloning site and a C-terminal BgIII cloning site
  SITE (1)..(2): HindIII cloning site
  SITE (3)..(22): flexible glycine/serine linker
  SITE (23)..(24): BglII cloning site
KLGGGGSGGGGSGGGGSGGGGSRS

SEQ ID NO: 96 - protein, 10 aa, Artificial Sequence
  His-Tag with flexible linker and Bsa1 cloning site
  SITE (1)..(2): Bsa1 cloning site
  SITE (3)..(4): flexible glycine serine linker
  SITE (5)..(10): His-tag
KLGSHHHHHH

SEQ ID NO: 97 - protein, 21 aa, Artificial Sequence
  N-terminal peptide for expression in CHO cells
  SITE (1)..(19): mouse Ig Kappa light chain signal sequence
  SITE (20)..(21): BamHI cloning site
METDTLLLWVLLLWVPGSTGS

SEQ ID NO: 98 - protein, 8 aa, Artificial Sequence
  His-tag with HindIII cloning site
  SITE (1)..(2): HindIII cloning site
  SITE (3)..(8): His-tag
KLHHHHHH

SEQ ID NO: 99 - protein, 9 aa, Apis mellifera
MVVYISYIY

SEQ ID NO: 100 - protein, 548 aa, Artificial Sequence
  4D5LH-L1-A21LH,  bispecific HER2 binding agent comprising a scFV antibody fragment binding domain 1 of HER2 and a a scFV antibody fragment binding domain 4 of HER2 connected by a glycine serine linker
MKKIWLALAGLVLAFSASADYKDDIGSDIVMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVELKRGGGGSGGGGSGGGGSGGGGSEVKLVESGGGLVQPGGSLRLSCATSGFNIKDTYIHWVRQAPGKGLEWVARIYPTNGYTRYADSVKGRFTISADTSKNTAYLQMNSLRAEDTAVYYCSRWGGDGFYAMDYWGQGTTVTVSSKLGGGGSRSDIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSADYYCVRSGNYEEYAMDYWGQGTSVTVSSKLGSHHHHHH

SEQ ID NO: 101 - protein, 21 aa, Apis mellifera
MKFLVNVALVFMVVYISYIYA

SEQ ID NO: 102 - protein, 250 aa, Artificial Sequence
  9.29_SH_G3 #2, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVSAQDKFGKTPGDLAGDNGNEWIAKKLLLAAAREGHREAVERAIKAGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 103 - protein, 254 aa, Artificial Sequence
  9.29_SH_G3 #6, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVFAQDKFGKTPFDLARDNGNEWIAKLLLAAALLEAARQGQRDRVEKLMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 104 - protein, 257 aa, Artificial Sequence
  9.29_SH_G3 #9, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVSAQDKFGKTPRDLARDNGNEWIWKLLLDALKYLLLEAAREGHRDRVEKLIKAGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 105 - protein, 258 aa, Artificial Sequence
  9.29_SH_G3 #10, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNAQDKFGKTPYDLATDNGNQWIAELLKRAALRRKLLEAARAGHRDEVEDLIKNGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 106 - protein, 259 aa, Artificial Sequence
  9.29_SH_G3 #11, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGALVKAKDKFGKTPKDLARDNGNQFIYELLEKAELLEKLLLEAAREGHRDRVEEFIKRGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 107 - protein, 260 aa, Artificial Sequence
  9.29_SH_G3 #12, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGAAVGAQDKFGKTPKDLARDNGNQWIYELLEKAEKDLRRKLLEAARAGHREEVEKLIKLGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 108 - protein, 261 aa, Artificial Sequence
  9.29_SH_G3 #13, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVNAQDKFGKTPEDLAKDNGNQWIAQKLEEAKKEDLDRKLLEAARAGHRDEVEDLIKNGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 109 - protein, 262 aa, Artificial Sequence
  9.29_SH_G3 #14, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVEAQDKFGKTPEDLAKDNGNQWIAQKLEEAKKKKDLDEKLLEAARAGHRDEVEDLIKNGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 110 - protein, 263 aa, Artificial Sequence
  9.29_SH_G3 #15, a bispecific HER2 binding agent comprising a DARPin binding domain 1 of HER2 and a a DARPin binding domain 4 of HER2 connected by a shared alpha helix
MRGSHHHHHHGSDLGKKLLEAARAGQDDEVRILMANGADVNAHDFYGITPLHLAANFGHLEIVEVLLKHGADVNAFDYDNTPLHLAADAGHLEIVEVLLKYGADVRAQDKFGKTPKDLARDNGNEWIRELLEKAERKLKDLDRKLLEAARAGHRDEVEDLIKNGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 111 - protein, 25 aa, Artificial Sequence
  artificial GGGGS linker with 5 repeats
GGGGSGGGGSGGGGSGGGGSGGGGS

SEQ ID NO: 112 - protein, 19 aa, Escherichia coli
MKKIWLALAGLVLAFSASA

SEQ ID NO: 113 - protein, 6 aa, Artificial Sequence
  FLAG-Tag M1
DYKDDI

SEQ ID NO: 114 - protein, 6 aa, Artificial Sequence
  6xHis-Tag
HHHHHH

SEQ ID NO: 115 - protein, 19 aa, Mus musculus
METDTLLLWVLLLWVPGST

SEQ ID NO: 116 - protein, 10 aa, Artificial Sequence
  Trastuzumab heavy chain CDR 1
GFNIKDTYIH

SEQ ID NO: 117 - protein, 17 aa, Artificial Sequence
  Trastuzumab heavy chain CDR 2
RIYPTNGYTRYADSVKG

SEQ ID NO: 118 - protein, 11 aa, Artificial Sequence
  Trastuzumab heavy chain CDR 3
WGGDGFYAMDY

SEQ ID NO: 119 - protein, 11 aa, Artificial Sequence
  Trastuzumab light chain CDR 1
RASQDVNTAVA

SEQ ID NO: 120 - protein, 7 aa, Artificial Sequence
  Trastuzumab light chain CDR 2
SASFLYS

SEQ ID NO: 121 - protein, 9 aa, Artificial Sequence
  Trastuzumab light chain CDR 3
QQHYTTPPT

SEQ ID NO: 122 - protein, 10 aa, Artificial Sequence
  A21 light chain CDR 1
GYSFTGYFIN

SEQ ID NO: 123 - protein, 17 aa, Artificial Sequence
  A21 heavy chain CDR 2
HISSSYATSTYNQKFQG

SEQ ID NO: 124 - protein, 11 aa, Artificial Sequence
  A21 heavy chain CDR 3
SGNYEEYAMDY

SEQ ID NO: 125 - protein, 17 aa, Artificial Sequence
  A21 light chain CDR 1
RSSQTLLYSNNQKNYLA

SEQ ID NO: 126 - protein, 7 aa, Artificial Sequence
  A21 light chain CDR 2
WAFTRKS

SEQ ID NO: 127 - protein, 9 aa, Artificial Sequence
  A21 light chain CDR 3
QQYSNYPWT

SEQ ID NO: 128 - protein, 107 aa, Artificial Sequence [sequence omitted]

SEQ ID NO: 129 - protein, 120 aa, Artificial Sequence [sequence omitted]

SEQ ID NO: 130 - protein, 107 aa, Artificial Sequence
  scFv Trastuzumab light chain variant 1
DIQMTQSPSSLSASVGDRVTITCRASQDVSTAVAWYQQKPGKAPKLLIYSASFLYSGVPSRFSGSRSGTDFTLTISSLQPEDFATYYCQQHYTTPPTFGQGTKVEIK

SEQ ID NO: 131 - protein, 120 aa, Artificial Sequence
  scFv trastuzumab heavy chain variant 1
EVQLVESGGGLVQPGGSLRLSCAASGFNIKDTYIHWVRQAPGKGLEWVARIYPTNAYTRYADSVKGRFTISADTSKNTAYLQMNSLRAEDTAVYYCSRWGGTGFYAMDYWGQGTLVTVSS

SEQ ID NO: 132 - protein, 120 aa, Artificial Sequence
  trastuzumab heavy chain variant 2
QVQLVQSGAEVKKPGASVKVSCKASGFNIKDTYIHWVRQAPGQGLEQMGRIYPTNGYTRYDPKFQGRVTITADTSSNTAYMELSSLRSEDTAVYYCSRWGGDGFYAMDYWGQGTLVTVSS

SEQ ID NO: 133 - protein, 120 aa, Artificial Sequence
  trastuzumab heavy chain variant 3
EVQLVQSGPELVQPGGSLRLSCAASGFNIKDTYIHWVKQAPGKGLEWISRIYPTNGYTRYDPSFKGRATISADTSSNTAYLQVNSLRAEDTAVYYCSRWGGDGFYAMDYWGQGTLVTVSS

SEQ ID NO: 134 - protein, 220 aa, Artificial Sequence
  A21 light chain
DIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 448 aa, Artificial Sequence
  A21 heavy chain with Fc
QVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 136 - protein, 223 aa, Artificial Sequence
  A21 heavy chain Fab portion
QVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSC

SEQ ID NO: 137 - protein, 220 aa, Artificial Sequence
  A21 light chain variant 1
DIVLTQSPDSLAVSLGERATINCRSSQTLLYSNNQKNYLAWYQQKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 223 aa, Artificial Sequence
  A21 heavy chain variant 1
QVQLVQSGAEVKKPGASVKVSCKASGYSFTGYFINWVRQAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 139 - protein, 220 aa, Artificial Sequence
  A21 light chain variant 2
DIVLTQSPDSLAVSLGERATINCRSSQPLEYSNNQWNYLAWYQKKPGQPPKLLISWAFTRKSGVPDRFSGSGSGTDFTLTISSLQAEDVAVYYCGQYSDYPNTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 448 aa, Artificial Sequence
  A21 heavy chain variant 2
QVQLVQSGAEVKKPGASVKVSCKASGYPFTQYFIHWVREAPGQGLEWMGHISSSYATVDYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 141 - protein, 220 aa, Artificial Sequence
  A21 light chain variant 3
DIQLTQSPSSLSASVGDRVTITCRSSQTLLYSNNQKNYLAWYQQKPGKAPKLLISWAFTRKSGVPSRFSGSGSGTDFTLTISSLQPEDFATYYCQQYSNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 223 aa, Artificial Sequence
  A21 heavy chain variant 3
EVQLVQSGPELVQPGGSVRISCAASGYSFTGYFINWVKQAPGKGLEWISHISSSYATSTYNQSFKGRATFSVDTSSSTAYMQLNSLRAEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 143 - protein, 222 aa, Artificial Sequence
  A21 heavy chain variant 4
VQLVESGGGLVQPGGSLRLSCAASGYSFTGYFINWVRQAPGKGLEWVSHISSSYATSTYNQSVKGRFTFSVDTSSSTAYLQMNSLRAEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 144 - protein, 219 aa, Artificial Sequence
  7C2 light chain
DIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 446 aa, Artificial Sequence
  A21 heavy chain
EVQLVQSGAEVKKPGASVKVSCKASGYSFTGYWMNWVRQAPGQGLEWIGMIHPLDAEIRANQKFRDRVTITVDTSTSTAYLELSSLRSEDTAVYYCARGTYDGGFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 146 - protein, 217 aa, Artificial Sequence
  H218 light chain
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 445 aa, Artificial Sequence
  heavy chain of antibody H218
EVQLVQSGAEVKKPGESLKISCKGSGYSFTSYWIGWVRQAPGQGLEWMGWISAYNGNTNYAQKLQGRVTMTTDTSTSTAYMELRSLRSDDTAVYYCAREGDGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 148 - protein, 220 aa, Artificial Sequence
  39s light chain
DIVMTQTPLSLSVTPGQPASISCKSSQSVFFRSNNKNILAWYLQKPGQPPQLLIYWASSRESGVPDRFSGSGSGTDFTLKISRVEAEDVGVYYCQQYFGSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 449 aa, Artificial Sequence
  39s heavy chain
EVQLVESGGGLVKPGGSLRLSCAASGFTFSSYSMSWVRQAPGKGLEWVSSISSSSSYIYYADSVKGRFTISRDNAKNSLYLQMNSLRAEDTAVYYCARGGDAYNYYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 150 - protein, 214 aa, Artificial Sequence
  MF3958 light chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 449 aa, Artificial Sequence
  MF3958 heavy chain
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPPVYYDSAWFAYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 152 - protein, 217 aa, Artificial Sequence
  Alternative Fc portion no mutation
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 217 aa, Artificial Sequence
  Alternative Fc part - Knob into hole - knob site V1
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 217 aa, Artificial Sequence
  Alternative Fc part - Knob into hole - hole site V1
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 155 - protein, 217 aa, Artificial Sequence
  Alternative Fc part - Knob into hole - knob site V2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 217 aa, Artificial Sequence
  Alternative Fc part - Knob into hole - hole site V2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 157 - protein, 469 aa, Artificial Sequence
  A21-TZB-441 light chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSDIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 448 aa, Artificial Sequence; identical to SEQ ID NO: 135
  A21-TZB-441 heavy chain

SEQ ID NO: 159 - protein, 223 aa, Artificial Sequence; identical to SEQ ID NO: 136
  A21-TZB-841 HC

SEQ ID NO: 160 - protein, 220 aa, Artificial Sequence; identical to SEQ ID NO: 134
  A21-TZB-241 light chain

SEQ ID NO: 161 - protein, 697 aa, Artificial Sequence
  A21-TZB-241 heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSQVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSK

SEQ ID NO: 162 - protein, 472 aa, Artificial Sequence
  A21-TZB-641 heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSQVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSC

SEQ ID NO: 163 - protein, 696 aa, Artificial Sequence
  A21-TZB-4oa light chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSDIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 164 - protein, 450 aa, Artificial Sequence
  A21-TZB-4oa heavy chain
QVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 165 - protein, 447 aa, Artificial Sequence
  A21-TZB-2oa light chain
DIVLTQSPDSLAVSLGERATINCRSSQTLLYSNNQKNYLAWYQKKPGQPPKLLISWAFTRKSGVPDRFSGSGSGTDFTLTISSLQAEDVAVYYCQQYSNYPWTFGQGTKVEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGESDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 166 - protein, 699 aa, Artificial Sequence
  A21-TZB-2oa heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSQVQLVQSGAEVKKPGASVKVSCKASGYSFTGYFINWVREAPGQGLEWMGHISSSYATSTYNQKFQGRVTFTVDTSSSTAYMELSSLRSEDTAVYYCVRSGNYEEYAMDYWGQGTLVTVSSASTKGPSVFPLAPSSKSTSGGTAALGCLVKDYFPEPVTVSWNSGALTSGVHTFPAVLQSSGLYSLESVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 167 - protein, 7 aa, Artificial Sequence
  Serine Alanine Linker
GAAGAAG

SEQ ID NO: 168 - protein, 6 aa, Artificial Sequence
  Gylcine Threonine Linker
GGTGGT

SEQ ID NO: 169 - protein, 5 aa, Artificial Sequence
  Serine Threonine Linker
STSTS

SEQ ID NO: 170 - protein, 5 aa, Artificial Sequence
  Proline Alanine Linker
PAPAP

SEQ ID NO: 171 - protein, 5 aa, Artificial Sequence
  Glycine Linker
GGGGG

SEQ ID NO: 172 - protein, 6 aa, Artificial Sequence
  Ovalabumin Linker
GSGSGS

SEQ ID NO: 173 - protein, 11 aa, Artificial Sequence
  Transthyretin Linker
GSGGGTGGGSG

SEQ ID NO: 174 - protein, 3 aa, Artificial Sequence
  Proline Linker
PPP

SEQ ID NO: 175 - protein, 21 aa, Artificial Sequence
  Proline Alanine Serine linker
ASPAAPAPASPAAPAPSAPAA

SEQ ID NO: 176 - protein, 21 aa, Artificial Sequence
  Proline Alanine Serine linker
ASAAAPAAASAAASAPSAAAA

SEQ ID NO: 177 - protein, 25 aa, Artificial Sequence
  Proline Alanine Serine linker
AASPAAPSAPPAAASPAAPSAPPAA

SEQ ID NO: 178 - protein, 6 aa, Artificial Sequence
  Proline Alanine Serine Linker
ASPASA

SEQ ID NO: 179 - protein, 9 aa, Artificial Sequence
  Proline Alanine Serine linker
ASPASPASA

SEQ ID NO: 180 - protein, 5 aa, Artificial Sequence
  XTEN-like-linker1
PAGSP

SEQ ID NO: 181 - protein, 5 aa, Artificial Sequence
  XTEN-like-linker2
STEPS

SEQ ID NO: 182 - protein, 5 aa, Artificial Sequence
  XTEN-like-linker3
STEEG

SEQ ID NO: 183 - protein, 5 aa, Artificial Sequence
  XTEN-like-linker4
GSAPG

SEQ ID NO: 184 - protein, 5 aa, Artificial Sequence
  ProB-linker1
GASTP

SEQ ID NO: 185 - protein, 5 aa, Artificial Sequence
  ProB-linker2
GPSAT

SEQ ID NO: 186 - protein, 7 aa, Artificial Sequence
  SA peptide linker
SAASAAS

SEQ ID NO: 187 - protein, 468 aa, Artificial Sequence
  construct 7C2LF light chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSDIVMTQSPDSLAVSLGERATINCRASQSVSGSRFTYMHWYQQKPGQPPKLLIKYASILESGVPDRFSGSGSGTDFTLTISSLQAEDVAVYYCQHSWEIPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0135700 A1
(43) Pub. Date: May 5, 2022

(54) Bispecific HER2 Ligands for Cancer Therapy

(71) Applicant: UNIVERSITÄT ZÜRICH, ZÜRICH (CH)

(72) Inventors: Andreas Plückthun, Zürich (CH); Florian Kast, Zürich (CH); Martin Schwill, Zürich (CH); Annemarie Honegger, Zürich (CH); Rastislav Tamaskovic, Rheinfelden (CH); Christian Jost, Zürich (CH)

(73) Assignee: UNIVERSITÄT ZÜRICH, ZÜRICH (CH)

(21) Appl. No.: 17/577,055

(22) Filed: Jan. 17, 2022

Related U.S. Application Data
(63) Continuation of application No. 16/153,857, filed on Oct. 8, 2018.
Application No. 16/153,857 is a continuation-in-part of application No. 14/430,224, filed on Mar. 22, 2015, now Pat. No. 10,093,740.

(30) Foreign Application Priority Data
Oct. 15, 2012 (EP) 12188598.2
Nov. 7, 2012 (EP) 12191673.8
Nov. 13, 2012 (EP) 12192465.8
Sep. 24, 2013 (EP) 13185724.5

(51) Int. Cl. C07K 16/32; A61P 35/00

(52) U.S. Cl. CPC C07K 16/32; C07K 2317/626; C07K 2317/31; C07K 2317/515; C07K 2317/52; C07K 2317/51; C07K 2317/24; C07K 2317/21; C07K 2317/622; C07K 2317/73; C07K 2317/92; C07K 2317/76; C07K 2318/10; C07K 2317/55; A61P 35/00

(57) ABSTRACT

The invention relates to a bispecific HER2-targeting agent that includes (a) a first polypeptide ligand that binds to HER2 extracellular domain 1, (b) a second polypeptide ligand that binds to HER2 extracellular domain 4, and (c) a linker covalently attaching said first polypeptide ligand to said second polypeptide ligand.

### CROSS-REFERENCE TO RELATED APPLICATIONS

This is a Continuation of U.S. patent application Ser. No. 16/153,857, filed Oct. 8, 2018, which is a Continuation-in-Part of U.S. patent application Ser. No. 14/430,224, filed Mar. 22, 2015, which issued as U.S. Pat. No. 10,093,740 on Oct. 9, 2018, which is the US National Stage of International Patent Application No. PCT/EP2013/071443, filed Oct. 14, 2013, which in turned claimed priority to European Patent Application Nos. 13185724.5, filed Sep. 24, 2013; 12192465.8, filed Nov. 13, 2012; 12191673.8, filed Nov. 7, 2012; and 12188598.2, filed Oct. 15, 2012. The contents of the foregoing patent applications are incorporated by reference herein in their entirety.

### FIELD

The present invention relates to bispecific targeting agents, particularly to antibodies, antibody fragments or other polypeptide ligands targeting HER2, and their use in cancer therapy.

### BACKGROUND

Epidermal Growth Factor Receptor 2 (HER2, ErbB2, Neu)

ErbB2/HER2/Neu is an orphan receptor tyrosine kinase, which preferentially dimerizes with other members of the epidermal growth factor receptor (EGFR) family (Yarden & Sliwkowski, 2001). HER2 has no known ligand, and its extracellular domain adopts a constitutively extended conformation, making HER2 the preferred heterodimerization partner for the ligand-activated receptors of the ErbB family (Franklin et al., 2004). Therefore, ErbB2 amplifies ligand-induced signaling of, e.g., ErbB3 receptor, by providing a scaffold for dimer formation and an active kinase domain for receptor transactivation. Overexpression of human epidermal growth factor receptor 2 (ErbB2/HER2/neu) is found in cancers of different tissue origin, such as on breast cancer (Slamon et al., 1987; Van de Vijver et al., 1988), prostate cancer (Minner et al., 2010), ovarian cancer (Tuefferd et al., 2007), gastric cancer (Rüschoff et al., 2007), adenocarcinomas (Reichelt et al., 2007) and non-small cell lung cancer (Mar et al., 2015). Overexpression of ErbB2 causes ErbB2 homodimer formation in cancer cells (Ghosh et al., 2011), and consequently, homodimers generate constitutive, ligand-independent, pro-proliferative and anti-apoptotic signaling (Junttila et al., 2009; Tamaskovic et al., 2016). HER2 is thus regarded as a non-autonomous amplifier of ErbB signaling that, additionally, enhances the affinity for ligands bound by other family members, attenuates receptor ubiquitination and increases receptor promiscuity by engaging a broader range of signaling adapters (Citri and Yarden, 2006; Jones et al., 2006). When overexpressed, HER2 spontaneously forms signaling-competent homodimers and ligand-independent heterodimers (FIG. 1A), thereby becoming a key regulatory signaling element driving cell proliferation, survival, migration and invasiveness of cancer cells (Tamaskovic et al., 2016; Junttila et al., 2009; Penuel et al., 2002). Overexpression of HER2 (ErbB2/Neu) may therefore be synonymous with highly aggressive and metastatic forms of cancer, and in particular, breast cancer (Slamon et al., 2001; Jackisch et al., 2014). Activated HER2 receptors trigger signaling pathways such as MAPK, PI3K/AKT, SRC/FAK, JAK/STAT and PKC (Hynes and MacDonald, 2009), but since each receptor recruits a specific set of phospho-Tyr-binding effector proteins, the individual ErbB members activate their own downstream signaling pattern (Jones et al., 2006; Schulze et al., 2005). For instance, within HER2-HER3 heterodimers, the phosphorylated cytoplasmic tail of HER3 strongly activates the PI3K/AKT survival pathway, whereas phosphorylated HER2 powerfully signals through the RAS/MAPK pathway. Since this combination of PI3K/AKT and RAS/MAPK signaling drives cell proliferation and survival, HER2/3 forms a potent oncogenic unit in HER2-addicted cancers (Holbro et al., 2003; Lee-Hoeflich et al., 2008).

Current Treatment of HER2-Positive Cancer

Monoclonal antibodies (mAbs) against HER2 with therapeutic efficacy target only few epitopes (Yip and Ward, 2002). The humanized mAb 4D5 (trastuzumab, Herceptin®) is directed against the membrane-proximal domain IV of HER2 (Cho et al., 2003). It specifically inhibits the growth of breast cancer cell lines addicted to HER2, inducing cell cycle arrest in G1 phase (Lane et al., 2000; Yakes et al., 2002) by inducing the dissociation of the ligand-independent HER2-HER3 heterodimers (Junttila et al., 2009), an action which is a likely component of the molecular action of trastuzumab. Another approved HER2-binding antibody, 2C4 (pertuzumab, Perjeta®), binds adjacent to the domain II dimerization arm, thereby disturbing the heterodimerization of HER2 with the other ligand-bound EGFR-family members (Franklin et al., 2004). Pertuzumab thus abrogates solely the ligand-stimulated growth, independent of HER2 overexpression. In fact, pertuzumab failed to show substantial effects on the proliferation of HER2-overexpressing breast cancer cells in vitro (Junttila et al., 2009), implying that the in vivo effects of pertuzumab are critically potentiated by mechanisms such as ADCC and CDC.

Since none of the known HER2-targeting mAbs is sufficient to trigger a robust cell death response in single-agent formats, they cannot fully exploit the addiction to oncogenic HER2 as a fragile point for therapeutic intervention. Importantly, the intra-ErbB pathway compensation through a number of feedback loops as well as other mechanisms rapidly neutralizes the perturbation caused by the approved antibodies, thereby leading to an acquired resistance against mAb treatment (Garrett and Arteaga, 2011). These obstacles have fostered the development of toxin-conjugated HER2-binding molecules that may give rise to a response in patients who failed trastuzumab therapy (Burris et al., 2011). Trastuzumab emtansine (T-DM1) (Kadcyla™), a maytansinoid conjugate, is thought to be endocytosed with the slow internalization and recycling rates intrinsic to HER2 and thus to release the toxin.

Although 60-70% of patients with HER2-positive metastatic breast cancer show initially high response rates to targeted anti-HER2 therapy (Valero et al. 2011, Bringolf et al., 2016), the majority of patient tumors develops cancer drug resistance within several months (Thery et al., 2014; Blackwell et al. 2012; O'Brien et al., 2010). Despite initial strong responses to the antibody therapy, trastuzumab treatment ultimately leads in the majority of treated patients to the development of acquired drug resistance (Esteva et al.; 2010). In view of the above mentioned state of the art, the objective of the present invention is to provide improved means and methods for targeting the HER2 protein for use in therapy of cancer. This objective is attained by the subject-matter of the independent claims.

### SUMMARY OF THE INVENTION

According to one aspect of the invention, a bispecific agent is provided, comprising
a. a first ligand that binds HER2 extracellular domain 1,
b. a second ligand that binds HER2 extracellular domain 4, and
c. a linker that connects said first ligand to said second ligand.

In some embodiments, the bispecific agent is a polypeptide. While the person skilled in the art can conceive of non-polypeptide targeting agents that can be rationally designed simply on the basis of the present specification, such as, by way of non-limiting example, RNA aptamers or L-RNA aptamers (see U.S. Pat. No. 6,605,713 and documents citing this publication), the majority of contemplated embodiments of the present invention relate to polypeptide ligands. For reasons of structural definition, the majority of these embodiments again are linked by a polypeptide linker as part of one single amino acid chain. While non-polypeptide bispecific agents are explicitly encompassed in the present invention, all embodiments mentioned herein below are to be read to explicitly include a polypeptide agent, particularly a single amino acid chain polypeptide agent.

In some embodiments, the bispecific agent is composed of a single sequence of amino acids. In some embodiments, the first ligand is connected to the second ligand covalently through a bridging moiety attached to amino acid side chains on the first and second ligands. In some embodiments, the first ligand is connected to the second ligand through a dimerization domain binding both the first ligand and the second ligand by non-covalent interactions.

According to an alternative to this aspect of the invention, a polypeptide is provided, comprising
a. a first binding site that binds HER2 extracellular domain 1,
b. a second binding site that binds HER2 extracellular domain 4, and
c. a linker that covalently links the first binding site and the second binding site.

The term “binding site” in the context of the present specification refers to the constituent parts, in particular the amino acid residues, of the first or second polypeptide ligand that in binding interact with particular constituent parts, for example a particular epitope, of the extracellular domain 1 or 4 of HER2.

According to another alternative of this aspect of the invention, a bispecific HER2-targeting agent is provided, comprising
a. a first polypeptide ligand that binds to HER2 extracellular domain 1 (Seq. ID 01),
b. a second polypeptide ligand that binds to HER2 extracellular domain 4 (Seq. ID 02) and
c. a linker covalently attaching the first polypeptide ligand to the second polypeptide ligand.

The term “bispecific” in the context of the present specification refers to the ability of the agent to specifically bind to two different epitopes of HER2.

“Binding” or “specifically binding” in the context of the present specification refers to the ability of the first (and respectively, second) polypeptide ligand to specifically and noncovalently attach to domain 1 (or, respectively, domain 4) of HER2 with a dissociation constant of equal or less than 10⁻⁷ M, 10⁻⁸ M or 10⁻⁹ M.

Domain 1 (SEQ ID 01) of HER2 (ErbB-2; Accession no. NP_004439.2) is the amino acid sequence

QVCT GTDMKLRLPA SPETHLDMLR HLYQGCQVVQ GNLELTYLPT
NASLSFLQDI QEVQGYVLIA HNQVRQVPLQ RLRIVRGTQL
FEDNYALAVL DNGDPLNNTT PVTGASPGGL RELQLRSLTE
ILKGGVLIQR NPQLCYQDTI LWKDIFHKNN QLALTLIDTN
RSRACHPCSP MCKGSRCWGE SSEDCQSLTR TVA.

Domain 4 (SEQ ID02) of HER2 (ErbB-2; Accession no. NP_004439.2) is the amino acid sequence

VNCS QFLRGQECVE ECRVLQGLPR EYVNARHCLP CHPECQPQNG
SVTCFGPEADQCVACAHYKD PPFCVARCPS GVKPDLSYMP
IWKFPDEEGA CQP

Accession numbers and Gene ID numbers refer to entries in the National Center for Biotechnology Information, Bethesda, Md.

UniProt. No refer to entries in the UniProt Knowledgebase.

ATCC numbers refer to entries in the American Type Culture Collection.

PDB IDs refer to entries in the protein data bank.

In some embodiments, the first polypeptide ligand or the second polypeptide ligand is an antibody, antibody fragment, or an antibody-like molecule.

In some embodiments, the antibody is an immunoglobulin consisting of two heavy chains and two light chains.

In some embodiments, the antibody fragment is a Fab fragment, i.e. the antigen-binding fragment of an antibody, or a single-chain variable fragment, i.e. a fusion protein of the variable region of heavy and the light chain of an antibody connected by a peptide linker. An antibody-like molecule in the context of the present specification refers to a molecule showing a specific binding to another molecule or target similar to the specific binding of an antibody. In some embodiments, the antibody-like molecule is a repeat protein, such as a designed ankyrin repeat protein (Molecular Partners, Zurich), a polypeptide derived from armadillo repeat proteins, a polypeptide derived from leucine-rich repeat proteins or a polypeptide derived from tetratricopeptide repeat proteins.

In some embodiments, the first polypeptide ligand and/or the second polypeptide ligand is selected from
a. an immunoglobulin Fab fragment,
b. an immunoglobulin scFv fragment, or
c. an immunoglobulin variable domain (domain antibody).

According to another aspect of the invention, a bispecific antibody is provided, which is selected from
a. an antibody, particularly an IgG, targeting HER2 domain 4 connected to a polypeptide ligand selected from an immunglobuline variable domain, Fab fragment, scFv Fragment and an ankyrin based polypeptide targeting domain 1 of HER2, wherein the polypeptide ligand is connected to
   i. the N-terminus of a heavy chain of the IgG,
   ii. the C-terminus of a heavy chain of the IgG,
   iii. the N-terminus of a light chain of the IgG or
   iv. the C-terminus of a light chain of the IgG, or
b. an antibody, particularly an IgG, targeting HER2 domain 1 connected a polypeptide ligand selected from an immunoglobulineimmunglobuline variable domain, Fab fragment, scFv Fragment and an ankyrin based polypeptide targeting domain 4 of HER2, wherein the polypeptide ligand is connected to
   i. the N-terminus of a heavy chain of the IgG,
   ii. the C-terminus of a heavy chain of the IgG,
   iii. the N-terminus of a light chain of the IgG or
   iv. the C-terminus of a light chain of the IgG.

The term “VL domain” in the context of the present specification refers to the variable domain of the light chain of an antibody.

Likewise, the term “VH domain” in the context of the present specification refers to the variable domain of the heavy chain of an antibody.

In some embodiments, a bispecific IgG is provided, consisting exclusively of a VH domain binding to domain 1 of HER2 and a VL domain binding to domain 4 of HER2 or exclusively of a VH domain binding to domain 1 of HER2, a VL domain binding to domain 4 of HER2 and a linker.

In some embodiments, the bispecific HER2-targeting agent of the invention is a bispecific IgG, consisting exclusively of an IgG targeting HER2 domain 4, where one or more of the structural loops of the Fc chain have been modified to bind to an epitope in HER2 domain 1 (see Wozniak-Knopp et al. (2010), Protein Engineering, Design and Selection 23, 289-297).

In some embodiments, the bispecific HER2-targeting agent of the invention is a bispecific IgG, consisting exclusively of an IgG targeting HER2 domain 1, where one or more of the structural loops of the Fc chain have been modified binding to an epitope in HER2 domain 4.

In some embodiments, the first polypeptide ligand and/or the second polypeptide ligand is an ankyrin repeat based polypeptide.

An ankyrin repeat based polypeptide in the context of the present specification refers to a polypeptide that comprises repetitive amino acid sequences, each repetitive sequence comprising two α-helices separated by loops.

In one embodiment, the antibody-like molecules are the Designed Ankyrin Repeat Proteins (DARPins) disclosed in US2012142611 (A1).

In some embodiments, the first polypeptide ligand comprises or is a sequence selected from the group composed of SEQ ID 10, SEQ ID 11, SEQ ID 12, SEQ ID 13, SEQ ID 14, SEQ ID 15, SEQ ID 16, SEQ ID 17, SEQ ID 18, SEQ ID 19, SEQ ID 20, SEQ ID 21, SEQ ID 22, SEQ ID 23, SEQ ID 24, SEQ ID 30, SEQ ID 31, SEQ ID 32, SEQ ID 33, SEQ ID 34, SEQ ID 35, SEQ ID 36, SEQ ID 37, SEQ ID 38, SEQ ID 39, SEQ ID 40, SEQ ID 41, SEQ ID 42, SEQ ID 43, SEQ ID 44, SEQ ID 45, SEQ ID 46, SEQ ID 47, SEQ ID 48, SEQ ID 49, SEQ ID 50, SEQ ID 61, SEQ ID 62, SEQ ID 63, SEQ ID 64, SEQ ID 65, SEQ ID 66, SEQ ID 93, SEQ ID 122 to SEQ ID 127, and SEQ ID 134 to SEQ ID 151, or functional equivalent having a sequence identity of at least 70%, 80%, 90%, 95%, or 98% to said sequence, particularly if the second polypeptide is an antibody targeting HER2 domain 43. Such polypeptide, which comprises or is a sequence described in the preceding paragraph, is an ankyrin repeat based polypeptide, an antibody fragment or an antibody that binds the extracellular domain 1 of HER2.

In certain embodiments, the first polypeptide ligand comprises a sequence selected from SEQ ID 122, SEQ ID 123, SEQ ID 124, SEQ ID 125, SEQ ID 126 and/or SEQ ID 127.

In some embodiments, the second polypeptide ligand comprises or is a sequence from the group composed of SEQ ID 25, SEQ ID 26, SEQ ID 27, SEQ ID 28, SEQ ID 29, SEQ ID 67, SEQ ID 68, SEQ ID 69, SEQ ID 92, and SEQ ID 116 to SEQ ID 121, and SEQ ID 128 to SEQ ID 133 or functional equivalent having a sequence identity of at least 70%, 80%, 90%, 95% or 98% to said sequence, particularly if the first polypeptide is an antibody targeting HER2 domain 1.

Such polypeptide, which comprises or is a sequence described in the preceding paragraph, is an ankyrin repeat based polypeptide, an antibody fragment or an antibody that binds the extracellular domain 4 of HER2.

In certain embodiments, the second polypeptide ligand comprises a sequence selected from SEQ ID 116, SEQ ID 117, SEQ ID 118, SEQ ID 119, SEQ ID 120 and/or SEQ ID 121.

Where reference is made herein to a polypeptide characterized by a particular sequence, such reference is meant to also encompass polypeptides having an identical function to the particular sequence, and showing a sequence identity of at least 70%, 80%, 90% or 95% to the certain sequence.

Identity in the context of the present invention is a single quantitative parameter representing the result of a sequence comparison position by position. Methods of sequence comparison are known in the art; the BLAST algorithm available publicly is an example.

In some embodiments, the first polypeptide ligand and the second polypeptide ligand are attached to each other by an oligopeptide linker, the first polypeptide, the second polypeptide ligand and the linker forming a continuous polypeptide chain.

One advantage of a bispecific HER2-targeting agent consisting of a continuous polypeptide chain is that such agent easily can be manufactured by recombinant biotechnology in a suitable host such as E. coli, yeast or mammal cells by expression of a single nucleotide sequence coding the continuous polypeptide chain.

In certain embodiments, the first polypeptide ligand is an antibody or antibody fragment and comprises or consists of a sequence selected from one of SEQ ID 134 to SEQ ID 142 and SEQ ID 144 to 151 or a functional equivalent polypeptide having a sequence identity of at least 70%, 80%, 90%, 95% or 98% to the aforementioned sequence.

In certain embodiments, the first polypeptide is an antibody or antibody fragment and comprises or consists of
a first sequence selected from one of SEQ ID 134, SEQ ID137, SEQ ID 139, SEQ and SEQ ID141 or a functional sequence having a sequence identity of at least 70%, 80%, 90%, 95% or 98% to the first sequence, and
a second sequence selected from one of SEQ ID 135, SEQ ID, 136, SEQ ID 138, SEQ ID140, SEQ ID 142 and SEQ ID 143, a functional sequence having a sequence identity of at least 70%, 80%, 90%, 95% or 98% to the second sequence.

In certain embodiments, the first polypeptide is an antibody or an antibody fragment and comprises or consists of
a first sequence characterized by SEQ ID EQ ID144 or a functional sequence having a sequence identity of at least 70%, 80%, 90%, 95% or 98% to the first sequence; and
a second sequence characteried by SEQ ID 145 or a functional sequence having a sequence identity of at least 70%, 80%, 90%, 95% or 98% to the second sequence.

In certain embodiments, the first polypeptide is an antibody or antibody fragment and comprises or consists of
a first sequence characterized by SEQ ID EQ ID146 a functional sequence having a sequence identity of at least 70%, 80%, 90%, 95% or 98% to the first sequence; and
a second sequence characteried by SEQ ID 147 a functional sequence having a sequence identity of at least 70%, 80%, 90%, 95% or 98% to the second sequence.

In certain embodiments, the first polypeptide is an antibody or antibody fragment and comprises or consists of
a first sequence characterized by SEQ ID EQ ID148 or a functional sequence having a sequence identity of at least 70%, 80%, 90%, 95% or 98% to the first sequence; and
a second sequence characteried by SEQ ID 149 or a functional sequence having a sequence identity of at least 70%, 80%, 90%, 95% or 98% to the second sequence.

In certain embodiments, the first polypeptide is an antibody or antibody fragment and comprises or consists of
a first sequence characterized by SEQ ID EQ ID150 or a functional sequence having a sequence identity of at least 70%, 80%, 90%, 95% or 98% to the first sequence and
a second sequence characteried by SEQ ID 151 or a functional sequence having a sequence identity of at least 70%, 80%, 90%, 95% or 98% to the second sequence.

In certain embodiments, the second polypeptide ligand is an antibody or antibody fragment and comprises or consists of a sequence selected from one of SEQ ID 128 to SEQ ID 133 or a functional equivalent polypeptide having a sequence identity of at least 70%, 80%, 90%, 95% or 98% to the aforementioned sequence.

In certain embodiments, the second polypeptide is an antibody and comprises or consists of
a first sequence selected from one of SEQ ID 128 and SEQ ID130 or a functional sequence having a sequence identity of at least 70%, 80%, 90%, 95% or 98% to the first sequence and
a second sequence selected from one of SEQ ID 129, SEQ ID, 131, SEQ ID 132, and SEQ ID133 a functional sequence having a sequence identity of at least 70%, 80%, 90%, 95% or 98% to the second sequence.

In some embodiments, the first polypeptide ligand is located at the N-terminus of the continuous polypeptide chain, the second polypeptide ligand is located at the C-terminus of the continuous polypeptide chain, and the linker is located between the first and the second polypeptide ligand. Embodiments wherein the agent of the invention is constituted by one continuous polypeptide chain offers advantages of production of the agent in a single step by methods of recombinant biotechnology, facilitating reproducibility of composition of the agent.

In some embodiments, the first polypeptide ligand and the second polypeptide ligand are attached covalently to each other by a bridging moiety or a crosslinker.

In some embodiments, the crosslinker connects a functionality such as an amino function on the side chain of lysine or a thiol function on a side chain of cysteine or the N-terminal amino group in the first polypeptide ligand to an amino acid side chain functional group in the second polypeptide ligand.

In some embodiments, the crosslinker is selected from glutaraldehyde, succinimide, tris[2-maleimidoethyl]amine, 1,4-bismaleimidobutane, and 1,4 bismaleimidyl-2,3-dihydroxybutane.

In some embodiments, a bispecific HER2-targeting agent according to the above aspects or embodiments of the invention is provided, wherein
a) the first polypeptide ligand partially or fully interacts non-covalently with
   i. a first D1 (domain 1) epitope, wherein the first D1 epitope comprises the amino acid residues E87, N89, Y90, L132, R135, D143, I145, W147, K148, L157, A158, L159, T160, L161 and I162 comprised within the amino acid sequence of HER2,
   ii. a second D1 epitope, wherein the second D1 epitope comprises the amino acid residues D88, A93, V94, I133, Q134, Q142, T144, L146, F151, H152, K153, N154, Q156 and D163 comprised within the amino acid sequence of HER2,
   iii. a third D1 epitope characterized by Seq. ID 55,
   iv. a fourth D1 epitope, wherein the fourth D1 epitope comprises the amino acid residues P100, L101, N102, N103, T104, R135, N136, P137, Y141, D143, T144, or
   v. a D1 epitope of domain 1 of HER2 (SEQ ID 01), wherein binding to the D1 epitope is competed by a polypeptide selected from SEQ ID 10, SEQ ID 11, SEQ ID 12, SEQ ID 13, SEQ ID 14, SEQ ID 15, SEQ ID 16, SEQ ID 17, SEQ ID 18, SEQ ID 19, SEQ ID 20, SEQ ID 21, SEQ ID 22, SEQ ID 23, SEQ ID 24, SEQ ID 30, SEQ ID 31, SEQ ID 32, SEQ ID 33, SEQ ID 34, SEQ ID 35, SEQ ID 36, SEQ ID 37, SEQ ID 38, SEQ ID 39, SEQ ID 40, SEQ ID 41, SEQ ID 42, SEQ ID 43, SEQ ID 44, SEQ ID 45, SEQ ID 46, SEQ ID 47, SEQ ID 48, SEQ ID 49, SEQ ID 50, SEQ ID 61, SEQ ID 62, SEQ ID 63, SEQ ID 64, SEQ ID 65, SEQ ID 66 and SEQ ID 93, and/or,
b) the second polypeptide ligand partially or fully interacts non-covalently with
   i. a first D4 (domain 4) epitope, wherein the first D4 epitope comprises the amino acid residues F512, E521, V524, L525, Q526, Y532, V533, N534, A535, R536, D549, G550, S551, V552, C554, F555 and V563 comprised within the amino acid sequence of HER2,
   ii. a second D4 epitope, wherein the second D4 epitope comprises the amino acid residues C522, R523, T553, C562 and A564 comprised within the amino acid sequence of HER2,
   iii. a third D4 epitope characterized by Seq. ID 56,
   iv. a fourth D4 epitope characterized by Seq. ID 57,
   v. a fifth D4 epitope, wherein the fifth epitope comprises the amino acid residues P557, E558, A559, D560, Q561, D570, P571, P572, F573, P595, D596, E597, E598, G599, A600, C601, Q602 and P603 comprised within the amino acid sequence of HER2, or
   vi. a D4 epitope of domain 4 of HER2 (SEQ ID 02), wherein binding to the D4 epitope is competed by a polypeptide having a sequence selected from SEQ ID 25, SEQ ID 26, SEQ ID 27, SEQ ID 28, SEQ ID 29, SEQ ID 67, SEQ ID 68, SEQ ID 69 and SEQ ID 92.

Non-covalent interactions in the context of the present specification include, without being restricted to, electrostatic interaction, hydrophobic interactions and van-der-Waals-interactions.

In some embodiments, the non-covalently interaction mediates the binding of the polypeptide ligand with a dissociation constant of equal or less than 10⁻⁷ M, 10⁻⁸ M or 10⁻⁹ M.

The term “epitope” in the context of the present specification refers to the part of the extracellular domain 1 or 4 of HER2 that is bound by the first or second polypeptide.

A polypeptide ligand is deemed to interact partially with an epitope in the context of the above definition if about 20%, 30%, 40%, 50%, 60%, 70%, 80%, or 90% of the indicated amino acid residues of the epitope, as laid out above, show interaction (e.g. hydrogen bond, van-der-Waals and similar non-covalent interaction) with the polypeptide ligand. Likewise, a polypeptide ligand interacts fully with an epitope, when all or at least about 95% of the indicated amino acid residues of the epitope show interaction with the polypeptide ligand.

In some embodiments, a bispecific HER2-targeting agent according to the invention is provided, wherein
a) the first polypeptide ligand is an ankyrin repeat based polypeptide, and the second polypeptide ligand is an antibody, an antibody fragment, or an antibody variable domain, or
b) the first polypeptide ligand is an antibody, an antibody fragment, or an antibody variable domain, and the second polypeptide ligand is an ankyrin repeat based polypeptide.

In some embodiments, a bispecific HER2-targeting agent according to the invention is provided, wherein the first polypeptide ligand is an antibody, an antibody fragment or an antibody variable domain, and the second polypeptide ligand is an antibody, an antibody fragment or an antibody variable domain.

In some embodiments, the linker has a length of equal or less than 65 Å, 60 Å, 55 Å, 50 Å, 45 Å, 40 Å, 35 Å, 30 Å, 25 Å, 20 Å, 15 Å, 10 Å or 5 Å.

In some embodiments, a bispecific HER2-targeting agent according to the above aspects or embodiments is provided, wherein
a) the first polypeptide ligand contacts the HER2 extracellular domain 1 through a D1 binding site,
b) the second polypeptide ligand contacts the HER2 extracellular domain 4 through a D4 binding site, and
c) the linker is selected to allow a direct spatial separation, or in other words a maximal distance between the D1 binding site and the D4 binding site of less than 80 Å, 75 Å. 70 Å, 65 Å, 60 Å, 55 Å, 50 Å, 45 Å, 40 Å, 35 Å, 30 Å, 25 Å, 20 Å, 15 Å, 10 Å or 5 Å.

In some embodiments, the linker consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids. In some embodiments, the linker consists of 1-10, 1-15, 1-20, 5-15, 5-10, 5-20, or 5-25 amino acids.

In some embodiments, the linker is a polyglycine/serine linker.

The term “polyglycine/serine linker” refers to a polypeptide linker that is composed of at least 50%, 60%, 70%, 80%, 90% or 100% of glycine and/or serine residues.

In some embodiments, the linker is characterized by an amino acid sequence (GGGGS)ₙ with n being 1, 2, 3, 4 or 5.

In some embodiments, the linker has one of the sequences SEQ ID 51, SEQ ID 52, SEQ ID 53, SEQ ID 54, SEQ ID 111, and SEQ ID 167 to SEQ ID 186.

In an alternative aspect of the present invention, a bispecific HER2-targeting agent is provided that comprises
a. a first polypeptide ligand that binds to HER2 extracellular domain 1,
b. a second polypeptide ligand that binds to HER2 extracellular domain 4 and
c. wherein said first polypeptide ligand and said second polypeptide ligand are covalently linked by a structural element common to said first polypeptide ligand and said second polypeptide ligand.

In other words, instead of having a flexible linker, the first and second ligands are rigidly connected by a sequence tract defined by structural motif of peptide secondary structure, wherein said connecting sequence tract is common to, or shared by, both of the ligands, such as, by way of non-limiting example, an alpha helix.

In some embodiments, the linker is formed by the C-terminus of the first polypeptide ligand and the N-terminus of the second polypeptide ligand, or the linker is formed by the C-terminus of the second polypeptide ligand and the N-terminus of the first polypeptide ligand.

In some embodiments, the linker is or comprises a secondary structure element, which is shared by the first polypeptide ligand and the second polypeptide ligand. In some embodiments, the shared structural element connecting the first polypeptide ligand and the second polypeptide ligand is an α-helix, in other words, the same alpha helix secondary structure motif is shared by the first polypeptide ligand and the second polypeptide ligand.

In some embodiments, the first polypeptide ligand is an ankyrin repeat based polypeptide, for example a “DARPin” as set forth in US20120142611 (A1), and the second polypeptide is also an ankyrin repeat based polypeptide or DARPin, and the C-terminal α-helix of the first polypeptide ligand and the N-terminal α-helix of the second polypeptide ligand together form a shared α-helix connecting the first polypeptide ligand and the second polypeptide ligand, or the C-terminal α-helix of the second polypeptide ligand and the N-terminal α-helix of the first polypeptide ligand form together a shared α-helix connecting the first polypeptide ligand and the second polypeptide ligand.

According to another aspect of the invention, a bispecific HER2-targeting agent is provided, wherein the bispecific HER2-targeting agent is characterized by a sequence selected from one of SEQ ID 157 to SEQ ID 166.

In certain embodiments, the bispecific HER2-targeting agent comprises or consists of
a first sequence characterized by SEQ ID 157 or a functional sequence having a sequence identity of at least 70%, 80%, 90%, 95% or 98% to the first sequence and
a second sequence characterized by SEQ ID 158 or a functional sequence having a sequence identity of at least 70%, 80%, 90%, 95% or 98% to the second sequence.

In certain embodiments, the bispecific HER2-targeting agent comprises or consists of
a first sequence characterized by SEQ ID 157 or a functional sequence having a sequence identity of at least 70%, 80%, 90%, 95% or 98% to the first sequence and
a second sequence characterized by SEQ ID 159 a functional sequence having a sequence identity of at least 70%, 80%, 90%, 95% or 98% to the second sequence.

In certain embodiments, the bispecific HER2-targeting agent comprises or consists of
a first sequence characterized by SEQ ID 160 or a functional sequence having a sequence identity of at least 70%, 80%, 90%, 95% or 98% to the first sequence; and
a second sequence characterized by SEQ ID 161 or a functional sequence having a sequence identity of at least 70%, 80%, 90%, 95% or 98% to the second sequence.

In certain embodiments, the bispecific HER2-targeting agent comprises or consists of
a first sequence characterized by SEQ ID 160 or a functional sequence having a sequence identity of at least 70%, 80%, 90%, 95% or 98% to the first sequence and
a second sequence characterized by SEQ ID 162 or a functional sequence having a sequence identity of at least 70%, 80%, 90%, 95% or 98% to the second sequence.

In certain embodiments, the bispecific HER2-targeting agent comprises or consists of
a first sequence characterized by SEQ ID 163 or a functional sequence having a sequence identity of at least 70%, 80%, 90%, 95% or 98% to the first sequence; and
a second sequence characterized by SEQ ID 164 or a functional sequence having a sequence identity of at least 70%, 80%, 90%, 95% or 98% to the second sequence.

In certain embodiments, the bispecific HER2-targeting agent comprises or consists of
a first sequence characterized by SEQ ID 165 or a functional sequence having a sequence identity of at least 70%, 80%, 90%, 95% or 98% to the first sequence; and
a second sequence characterized by SEQ ID 166 or a functional sequence having a sequence identity of at least 70%, 80%, 90%, 95% or 98% to the second sequence.

In certain embodiments, the bispecific HER2-targeting agent comprises or consists of
a first sequence characterized by SEQ ID 187 or a functional sequence having a sequence identity of at least 70%, 80%, 90%, 95% or 98% to the first sequence; and
a second sequence characterized by SEQ ID 145 or a functional sequence having a sequence identity of at least 70%, 80%, 90%, 95% or 98% to the second sequence.

According to another aspect of the invention, an isolated nucleic acid molecule is provided, wherein the isolated nucleic acid molecule encodes a bispecific HER2-targeting agent according to any one of the above aspects or embodiments of the invention.

According to another aspect of the invention, a bispecific HER2-targeting agent according to any of the above aspect or embodiments of the invention is provided for use in a method for preventing or treating malignant neoplastic diseases.

According to another aspect of the invention, a bispecific HER2-targeting agent according to any of the above aspect or embodiments of the invention is provided for use in a method for preventing or treating malignant neoplastic diseases, wherein the disease is characterized by cells overexpressing HER2.

A disease characterized by cells overexpressing HER2 or a HER2-positive disease is defined in the context of the present specification to be present if a high HER2 (protein) expression level is detected by immunohistochemical methods, by flow-cytometric methods such as FACS, or as HER2 gene amplification, for example a HER2 gene copy number higher than 4 copies of the HER2 gene per tumor cell, or by a combination of these methods, in samples obtained from the patient. One example of such disease is often breast cancer, where cells overexpressing HER2 can be cells obtained from breast tissue biopsies or breast tissue resections or in tissue derived from metastatic sites. One frequently applied method for detecting HER2 overexpression and amplification at the gene level is fluorescence in situ hybridization (FISH), which is also described in US2003/0152987 to Cohen et al.

In some embodiments, a cell overexpressing HER2 is characterized by at least 2, 4, 6, 8, 10, 15, 20 or 25 copies of the HER2 gene (ERBB2 gene, Gene ID: 2064) in the nucleus in a FISH (fluorescence in-situ hybridization) assay.

In one embodiment, the copy number of the HER2 gene is measured by fluorescence in situ hybridization.

In one embodiment, a cell overexpressing HER2 is characterized by at least 2, 4, 6, 8, 10, 15, 20 or 25 signals per nucleus in a fluorescence in situ hybridization assay.

According to yet another aspect of the invention, a method is provided for treating a patient suffering from malignant neoplastic disease, comprising the administration of a bispecific agent according to any of the above specified aspects or embodiments of the invention to said patient.

In some embodiments, the malignant neoplasitic disease is a carcinoma of the stomach, endometrium, salivary gland, lung, kidney, colon, thyroid, pancreas or bladder.

### BRIEF DESCRIPTION OF DESCRIBED SEQUENCES

The nucleic and amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named 95083_303_1402_seqlist, about 360 KB, which is incorporated by reference herein.

### BRIEF DESCRIPTION OF THE FIGURES

FIGS. 1a-c show the increased anti-tumor activity of bispecific targeting agents in cell proliferation assays. The Y axis shows cell viability in different cell lines expressing HER2 after treatment with any of the agents identified in the legend.

FIG. 2 shows the quantification of cellular DNA content by flow cytometry in absence and presence of different anti-tumor agents.

FIGS. 3a-b show the induction of apoptosis by bispecific targeting agents quantified by terminal transferase dUTP nick end labeling (TUNEL) assays and flow cytometry.

FIG. 4 shows the Western blot analysis of HER2/HER3 signaling pathway, PI3K/AKT and MAPK pathway and downstream targets of cell cycle and apoptosis.

FIGS. 5a-b show quantitative western blot analyses of the treatment time course measuring HER2/HER3 receptor expression and phosphorylation after treatment with anti-HER2 binding agents.

FIG. 6 shows the inhibition of ligand-stimulated growth by bispecific targeting agents in cell proliferation assays.

FIG. 7 shows the pictorial summary of anti-HER2 targeting formats.

FIG. 8 shows the anti-tumor activity of bispecific binding reagents quantified by cell proliferation assays, shown is the effect of different concentrations of anti-tumor agents on the cell viability.

FIG. 9 shows the anti-tumor activity of all constructs that share a similar epitope on domain I of ECD HER2 in a cell proliferation assay, the Y-axis showing the viability of BT474 cells after treatment with any of the agents identified in the legend.

FIG. 10 shows the anti-tumor activity of single binding agents. The Y-axis shows the viability of BT474 cells after treatment with any of the agents identified in the legend.

FIG. 11 shows the effect of combination treatment of the single anti-HER2 binding agents on the cell viability (Y-axis) of BT474 cells after treatment with any of the agents identified in the legend.

FIGS. 12a-e show the effect of different anti-tumor agents on the viability of trastuzumab-resistant cell lines in cell proliferation assays, the Y-axis showing the viability of the cell lines determined by absorbance of reduced XTT after treatment with any of the agents identified in the legend.

FIGS. 13a-b show the effect of trastuzumab and pertuzumab on the anti-tumor activity of the bispecific targeting agents in cell proliferation assays. Data presentation as in FIG. 11.

FIG. 14 shows the anti-tumor activity of different anti-tumor agents in cell proliferation assays. Data presentation as in FIG. 11.

FIG. 15 shows the results of an ELISA with bispecific targeting agents and pertuzumab. The Y-axis shows the concentration of the agents identified in the legend bound to HER2 in presence of pertuzumab.

FIG. 16 shows the competitive binding of G3 and H14 with trastuzumab. The Y-axis shows the percent binding of the agents identified in the legend to domain 4 of HER2 in presence of trastuzumab.

FIGS. 17A-D show the binding affinity, binding stoichiometry and binding mode of single binding units and bispecific binding agent to HER2 on the surface of cancer cells; A and B, on-rate determination of single binding agents; C, on-rate determination of bispecific binding agents; D, off-rate determination of single and bispecific binding agents; MFI mean fluorescence intensity.

FIGS. 18A-G show the dissociation from the surface of BT474 cells (A) and the anti-tumor activity (B-G) of single binding agents and bispecific binding agents. A: median fluorescence intensities of fluorescently labeled agents bound to the BT474 surface are plotted as function of dissociation time; B-G: The Y-axes show the viability of BT474 (B-D, F,G) or MCF7 (E) cells after treatment with any of the agents identified in the legend.

FIG. 19 shows the construction principle of the A21H_4D5LH_A21L (top, “A”) and a cartoon of the complete diabody construct as expressed in CHO cells (bottom, “B”). Here “heavy chain” refers to the VH domain, “light chain” to the VL domain.

FIG. 20 shows the effect of different anti-tumor agents on the viability of BT474 cells in cell proliferation assays, the Y-axis showing the viability of the cell lines determined by absorbance of reduced XTT after treatment with any of the agents (100 nM) identified in the legend. Data were normalized to the control, which was set to 100%.

FIG. 21 shows the effect of different anti-tumor agents on the viability of HCC1419 cells in cell proliferation assays, the Y-axis showing the viability of the cell lines determined by absorbance of reduced XTT after treatment with any of the agents (100 nM) identified in the legend. Data were normalized to the control, which was set to 100%.

FIG. 22 shows the induction of apoptosis in BT474 cells by bispecific targeting agents quantified by terminal transferase dUTP nick end labeling (TUNEL) assays and flow cytometry.

FIG. 23 shows the Western blot analysis of apoptosis as detected by the cleavage of Poly ADP Ribose Polymerase (PARP). GAPDH is a loading control.

FIG. 24 shows the effect of different anti-tumor agents on the viability of BT474 cells in cell proliferation assays, the Y-axis showing the viability of the cell lines determined by absorbance of reduced XTT after treatment with any of the agents identified in the legend.

FIG. 25 shows the signaling scheme summarizing the mechanisms of action of trastuzumab, pertuzumab and biparatopic anti-HER2 binding agents and relevant downstream pathways. The EGFR family shows a broad potential to activate various downstream pathways, including RAS/MAPK, PI3K/AKT/mTOR and SRC/FAK1/NFkB signaling pathways.

FIG. 26 shows schemes of preferred biparatopic anti-HER2 binding agents.

FIG. 27 shows the viability testing of CHOs during the expression of construct 441 (scFV-IgG). Expression optimization of construct 441 in CHOs cells for indicated time. Cells were cultured in CHOgro medium from Mlrus (MIR 6260) and additionally fed with free cysteine (reduced form) (2), glutathione (3), fetal calf serum (4) or all additives respectively (5). CHO cells were analyzed on CASY cell counter (Schärfe System).

FIG. 28 shows a Western blot of construct 441 expression, secreted to the medium of CHO cells after indicated times. Cells were cultured in CHOgro medium from Mlrus (MIR 6260) (1) and additionally fed with free cysteine (reduced form) (2), glutathione (3), fetal calf serum (4) or all additives together (5), respectively. Protein was precipitated from medium by acetone precipitation and re-solubilized in SDS PAGE buffer. Proteins were resolved on 4-12% gradient gel and the western blot was analyzed on an Odyssey system (LI-COR). Purified intact full length construct 441 is shown as control (A) and runs above the 170 kDa marker. Molecular weight marker Page ruler from Thermo Scientific is shown in red.

FIG. 29 shows vector map for the expression plasmid of bispecific constructs (Pymex10 based vectors with double expression cassette [CMV GOI polyA]).

FIG. 30 shows cell proliferation assays (XTT) with BT474 cells after 4 days of treatment. Trastuzumab (TZB), biparatopic DARPin (6L1G) and different fusion variants of the biparatopic construct. LF IgG HL (murine parent of construct 441), HF IgG HL (murine parent of construct 241) show similar anti-proliferative activity compared to the biparatopic DARPin 6L1G, which is superior to trastuzumab (TZB). HF IgG LH (murine variant, no seq.) and LF IgG LH (murine variant, no seq.) show reduced anti-proliferative activity compared to biparatopic DARPin and higher IC₅₀ concentrations.

FIG. 31 shows cell proliferation assays (XTT) with BT474 cells after 4 days of treatment to test the effect of the linker length. Biparatopic DARPin (6L1G) and different fusion linker variants of the biparatopic construct (murine parent construct of 441) are compared. The 2-AA linker (GS) shows highest anti-proliferative activity. The 4-, 7- and 12-AA linkers show similar activity. The 22-AA linker variant shows reduced activity.

FIG. 32 shows cell proliferation assays (XTT) with BT474 cells after 4 days of treatment. Biparatopic DARPin (6G; 6L1G), biparatopic construct 441 (441), biparatopic construct 411 (humanized kappa1 VH1) and biparatopic construct 443 (humanized kappa4 VH3). All show similar plateau levels of anti-proliferative activity, except 443, which shows reduced activity.

FIG. 33 shows cell proliferation assays (XTT) with BT474 cells after 4 days of treatment with different humanized versions of A21 IgG, when fused to TZB scFv. The strategy of humanization is described above. Different variants use humanized kappa1 VH3 or a humanized kappa1 VH core graft.

FIG. 34 shows XTT cell proliferation assay with BT474 cells after 4 day of treatment. Tetravalent IgG (HF IgG HL and LF IgG HL murine) versus bivalent Fab fusions (HF Fab HL and LF Fab HL murine). All constructs show similar plateau and IC50 values.

FIG. 35 shows XTT cell proliferation assay with SKBR3 cells after 4 day of treatment. Biparatopic DARPin (6G) biparatopic construct (441 tf), trastuzumab (TZB).

FIG. 36 shows cell proliferation assays (XTT) with CALU-3 cells after 4 days of treatment. Biparatopic DARPin (6G), biparatopic construct (construct 441 (441tf) (SEQ ID Nos. 157, 158)), trastuzumab (TZB).

FIG. 37 shows cell proliferation assays (XTT) with BT474 cells after 4 days of treatment, testing effect of domain 1 binding unit. Biparatopic construct with A21 (construct 441tf (SEQ ID Nos. 157, 158)) or 7C2 fusions show different IC50 and plateau level.

FIG. 38 shows cell proliferation assays (XTT) with BT474 cells after 4 days of treatment, testing the effect of domain 1 binding unit. Biparatopic construct with A21 (construct 441) or with 39S (39s HF IgG H)L

FIG. 39 shows XTT cell proliferation assays with HCC1419 cells after 4 days of treatment. Biparatopic DARPin (6G; 6L1G), biparatopic construct 441 (441tf) and bivalent LF-oaFabFc (A21-TZB-4oa). 441 and 6G show similar inhibition of cell proliferation after 4 days. LF-oaFabFc show slightly reduced inhibition of cell proliferation compared to 441.

FIG. 40 shows XTT cell proliferation assay with BT474 and HCC1419 cells after 4 day of treatment. All human.

FIG. 41 shows XTT cell proliferation assay with BT474 and HCC1419 cells after 4 day of treatment. All human.

FIGS. 42A-B show a) in the upper panel XTT cell proliferation assays with BT474 (left) and HCC1419 (right) cells after 4 day of treatment; and in the lower panel XTT cell proliferation assays with BT474 (left) and HCC1419 (right) cells after 4 day of treatment (variants with higher affinity (NGS and GGG)); b) repeated experiments with a new expression of NGS.

FIG. 43 shows XTT cell proliferation assays with BT474 (left) and HCC1419 (right) cells after 4 day of treatment.

FIG. 44 shows XTT cell proliferation assays with HCC1419 cells grown as 3D spheroids.

FIG. 45 shows Western Blots 24 hours post treatment (BT474) with indicated agents (murine).

FIG. 46 shows in the upper panel Induction of apoptosis in BT474 cells after 3 days of treatment. Average number of propidium iodide (P1) positive cells was determined for 4 replicates, counted by cell profiler and was analyzed with Student's t-test. Biparatopic construct (441, 441tf) induced significantly more cell death than trastuzumab (TZB). 441 and biparatopic DARPin (6L1G) show similar level of cell death; and in the lower panel Induction of apoptosis in BT474 cells after 3 days of treatment. Average number of annexin-V positive cells was determined for 3-4 replicates, counted by cell profiler and was analyzed with Student's t-test. Biparatopic construct 441 induced significantly more apoptosis than trastuzumab (TZB). Construct 441 and 6L1G show similar level of apoptosis.

FIG. 47 shows images of BT474 cells treated with the indicated agents for 3 days.

FIG. 48 shows Alexa647-labeled trastuzumab (TZB), biparatopic construct 441 and biparatopic one armed constructs oaLF and oaHF were incubated for 1 h at 100 nM concentration with 3 million BT474 cells in 3 ml PBS containing NaN₃ (0.1%) and BSA (1%) at 4° C. Note that BT474 cells were pre-treated with 0.1% NaN₃ in PBS with 1% BSA to block internalization before binding. Cells were analyzed afterwards on CyFlow Space instrument (Partec). All binding agents show specific binding to the surface of HER2-positive BT474 cells.

FIG. 49 shows the induction of cell death after treatment with 100 nM of indicated agents. BT474, N87, HCC1419 and SKBR3 cells were seeded 24 h before treatment in 96 black clear-well microscopy plates (Nunc), continuously treated for 3 days and stained with HOECHST-33342 (Invitrogen) for total cells and with propidium iodide (Sigma) for membrane-permeable dead cells. Cells were analyzed on a Lionheart FX Automated Microscope (BioTek Instruments) and the number of propidium iodide and HOECHST-33342 positive cells was quantified with Gen5 software (BioTek Instruments). The ratio of propidium iodide and HOECHST-33342 positive cells was calculated for 3 biological replicates and the mean and SD is shown in the corresponding column plots. Biparatopic binding agents (6L1G, 441, 841, LFoa, 241, 641, HFoa, 7C2LF) binding to domain 1 and 4 of HER2 induce continuously more dead cells than trastuzumab (TZB) or the combination of trastuzumab and pertuzumab (TZB+PZB) in HER2-positive cancer cells.

### DETAILED DESCRIPTION OF CERTAIN EMBODIMENTS

The Principle of Anti-Tumor Activity of Bispecific Targeting Agents

The minimal setup of a bispecific targeting agent of the invention is composed of 3 units. Firstly, the bispecific binding agent comprises a binding unit targeting domain 1 of the extracellular domain (ECD) of HER2. Secondly, the bispecific binding agent comprises a binding unit targeting domain 4 of the ECD of HER2. Thirdly, the bispecific binding agent comprises a linker unit or linker in-between the binding unit targeting domain 1 of HER2 and the binding unit targeting domain 4 of HER2, whose optimal length depends on the nature of both binding units.

In some embodiments, the linker or linker unit is a polypeptide linker.

In some embodiment, the linker is a polyglycine/serine linker. Such linker has the advantage that it is highly soluble in water, has a flexible fold, is resistant against proteolysis and adopts either a random coil or an extended structure.

In some embodiments, the linker is a short linker composed of the amino acids: GGGGS (G₄S). Bispecific constructs comprising 1 to 4 repeats of G45 show superior anti-tumor activity. Bispecific constructs comprising 5 or more repeats of G45 show decreasing anti-tumor activity with longer linker length. Other amino acid compositions might be used to connect the binding units.

In some embodiments, the linker or linker unit comprises flexible regions of binding scaffolds described above or is a chemical cross-linker, wherein both binding units are covalently connected by the linker. A chemical cross-linker in the context of the present specification refers to a compound capable of covalently connecting the first and the second polypeptide ligand of the invention. Examples for such chemical crosslinkers include, without being restricted to, glutaraldehyde, bissulfosuccinimidyl suberate, carbodiimide, bis(succinimidyl)penta(ethylene glycol), bis(succinimidyl) nona(ethylene glycol), bis(sulfosuccinimidyl) suberate, dimethyl suberimidate, an ethylene glycol characterized by formula (—CH2OH—CH2OH—)ₙ, wherein n is 2, 3, 4, 5, 6, 7, 8, 9, 10, 11, 12, 13, 14, 15, 16, 17, 18, 19, 20, 21, 22, 23, 24 or 25 and one or both termini of the ethylene glycol are substituted by a succinimide or maleimide group, N-(κ-Maleimidoundecanoyloxy) sulfosuccinimide ester, sulfosuccinimidyl (4-iodoacetyl) aminobenzoate, 1,8-bismaleimidodiethyleneglycol and 1,11-bismaleimidotriethyleneglycol.

In some embodiments, the linker or linker unit is a dimerization domain or additional functional units inducing the dimerization of both binding units to connect both epitopes on HER2 or, in other words, dimerization domains.

A dimerization domain in the context of the present specification refers to a functional unit consisting of two polypeptides that are capable of specific binding to each other or dimerizing. The two polypetides may be part of the same polypeptide chain. Non-limiting examples for such dimerization domains are leucine zipper domains such as in GCN4 (UniProt. No. P03069), helix-helix domains, dimerization domains composed of beta-sheets, coiled coil helices such as in c-Jun (Uniprot. No. P05412) or c-Fos (Uniprot. No P01100), helix bundles like in the dimerization domain of the mip protein (Uniprot. No Q70YI1), helix-turn-helix motifs such as in the repressor protein cl (Uniprot. No. P03034) and antibody Fc regions.

Such linker unit may determine the anti-tumor activity of the bispecific targeting agent. The single binding units used in the examples disclosed here have no or only weak anti-tumor activity as single agents.

In some embodiments, linkers of other composition can be used, provided they bring said binding domains into a disposition leading to apoptosis in the targeted cell, as can be assayed by the methods provided herein.

The term “flexible linker” in the context of the present specification refers to a polypeptide connecting the first polypeptide ligand and the second ligand that is characterized by a random coil conformation or extended structure. A flexible linker may further be characterized by the absence of secondary structures such as helices or β-sheets or a maximal secondary structure content of 10%, 20% 30% or 40%.

The term “overlapping epitope” in the context of the present specification refers to an epitope that is partially identical to a certain epitope.

In some embodiments, binders to the most preferred epitopes are generated in using the display methods described above (phage display, ribosome display or yeast display). The DARPins 926, 929 or G3, whose sequences are disclosed in SEQ ID 14, SEQ ID 15, SEQ ID 16, SEQ ID 17, SEQ ID 18 SEQ ID 19, SEQ ID 20, SEQ ID 21, SEQ ID 25, SEQ ID 61, SEQ ID 62, SEQ ID 63 and SEQ ID 64 can be used as competitors. Their genes can be synthesized and they can be expressed and purified as detailed in Zahnd et al. (2007) J. Mol. Biol. 369, 1015-1028. When the pool of binders selected in ribosome display or in phage display to the HER2 domains immobilized on magnetic beads or in microtiter plates are exposed to the competing DARPins, the binders will be preferentially eluted which show the same epitope.

In one embodiment, the mode of binding for one bispecific molecule, constructed according to the invention, is intermolecular. The linker unit in the bispecific agents determines the mode of binding. To be more precise, the length of the linker, and the orientation imparted on the binding domains by the attachment points of the linker influence whether the bispecific molecule binds in an intermolecular way, i.e. connecting two HER2 molecules. Hence, upon binding on a cell, the bispecific agents connect domain 1 of one HER2 receptor molecule with the domain 4 of another HER2 receptor molecule. In some embodiments, the connection between both epitopes bound by the binding units of particularly active bispecific constructs is bridged by a short linker (5 amino acids or approx. 15 Å).

In the structure of the whole extracellular domain of HER2 (PDB ID: 1N8Z) (Cho H S, et al. (2003), Nature 421:756-760), the distance between the epitope on domain 1 and the epitope on domain 4 is at least 80 Å long, and it is thus impossible that the bispecific molecule binds in an intramolecular way to this structure of HER2 (i.e., the domain 1 binding moiety and the domain 4 binding moiety cannot bind to domains 1 and 4 of one and the same HER2 molecule).

Domain 4 of the HER2 receptor is close to the transmembrane helix of the HER2 receptor and therefore restricted in its motional freedom. Domains 1, 2 and 3 are connected to domain 4 by flexible hinges. As it is known for other EGFR receptors, domains 1, 2 and 4 can change their relative orientation upon ligand binding. The conformational change in other EGFR receptors occurs from a state where domain 2 and 4 are in direct contact and domain 1 and 3 are separated (tethered conformation) to a state where domain 2 and 4 separate and domain 1 and 3 are connected via the respective ligand (Mark A. Lemmon, Ligand-induced ErbB receptor dimerization, Experimental Cell Research, 315(4), 2009, Pages 638-664). However, even in the tethered conformation, the distance between domain 1 and domain 4 remains too large to be compatible with a 15 Å linker. Furthermore, the “tethered” conformation is thought to be absent in HER2, due several findings like e.g. the absence of stabilizing amino acids in the domain 4 contact region (e.g. G563 and H565 of HER3 are replaced with P and F) found in the crystal structure of HER2 (Cho et al., 2003 Nature 421: 756-760).

Hence, without wishing to be bound by theory, a conformation is postulated which is induced or stabilized by the bispecific targeting agents of the invention. This conformation is referred in the following as the stabilized inactive HER2 homodimer conformation. These stabilized inactive homodimers of HER2 may also exist in the context of larger HER2-HER2 interaction units like e.g. trimers, tetramers or up to HER2 clusters. The examples shown herein demonstrate that, in certain embodiments of the present invention, key tyrosine residues on the intracellular part of HER2 at the “phosphorylation tail” and in the kinase domain become dephosphorylated upon treatment with the bispecific targeting agents, while total HER2 levels remain quite constant in cancer cells that have not yet undergone apoptosis.

In certain embodiments, the stabilization of inactive HER2 homodimers by the bispecific targeting agents disclosed in the present invention consequently inhibits other HER2 interactions, e.g. with HER3. HER2 and HER3 receptor form a heterodimer with strong oncogenic, anti-apoptotic signaling. As a consequence of both inhibition of HER2 phosphorylation and HER3 phosphorylation, both downstream pathways PI3K-AKT and MAPK-ERK, and possibly other signaling pathways, become persistently inactivated and or down-regulated. Both pathways are down-regulated to such an extent that the pro-apoptotic protein BIM becomes increasingly expressed in the cancer cells, leading to caspase activation and finally apoptosis.

Delineation of the Invention: Design Criteria of Active Bispecific Molecules.

While the examples provided relate to the DARPins 9.26 or 9.29 linked to the DARPins G3 or H14 by a short flexible linker, a person skilled in the art can replace, in light of the information provided herein, any or both of said DARPins by other scaffolds or antibody Fab fragments or antibody scFv fragments or antibody domains, binding to an overlapping epitope on domain 1 or domain 4, respectively. If the orientation of the binding protein is not known from structural modeling or experimental structure determination, both linkages (BinderA-FL-BinderB and BinderA-FL-BinderB) can be readily constructed and tested in light of the information provided herein. The modular principle of the bispecific targeting agent makes it thus facile for the person skilled in the art to replace single parts in the construct by other binding or linking units.

Bispecific HER2 Targeting

The present invention is based on a binding molecule that functions as a HER2-specific molecular crosslinker, which leads to the formation of inactive HER2 homodimers, instead of inhibiting HER2 dimerization. The mechanism of action of the targeting molecule of the invention is thus radically different from the HER2-directed therapies so far described. The agents of the invention lead to HER2 homodimers being linked in such way that they become signalling-inactivated. The examples shown herein demonstrate the dephosphorylation of key tyrosine residues of the intracellular part of HER2. Hence, the so induced HER2 homodimers show a strongly reduced downstream signalling via the MAPK pathway, which is directly shown by the dephosphorylation of the MAP-kinase extracellular-signal regulated kinase 1 and 2 (Erk1/2).

In addition, these inactive HER2 homodimers fail to interact, in some embodiments, with other members of the EGF receptor family, most importantly with HER3. HER2-HER3 interactions and the corresponding phosphatidylinositol 3-kinase protein kinase B (PI3K-PKB, alternatively called PI3K-AKT) signalling pathway are known to drive cell proliferation and inhibit apoptosis in HER2-overexpressing cancer cells.

In still other embodiments, by preventing HER2-HER3 interactions by the stabilization of inactive HER2 homodimers, the downstream pathway PI3K-AKT becomes also inhibited. Hence, dephosphorylation of AKT was shown to result from application of the molecules of this invention. The simultaneous inhibition of both pathways, to a higher extent than achieved by the application of trastuzumab or pertuzumab or their combined action, stimulates, in yet other embodiments, the expression of Bcl-2-like protein 11 (BIM). The expression of BIM, mainly the short isoform BIMₛ, finally leads, in certain embodiments, to the induction of the cell's intrinsic apoptotic program. As shown, the mode of action of the bispecific targeting agents is not the sum of actions of known molecular formats, because the building blocks, the single binding units, do not necessarily need to have anti-tumor activity by themselves. However, the connection of both disclosed epitopes in a preferentially intermolecular manner of preferred geometric disposition generates the potent anti-tumor agent.

Disclosed herein are two epitopes that may be bound by the HER2 targeting molecule, at the level of single amino acids of the HER2 extracellular domain, which are derived from multiple crystal structures of HER2 in complex with the respective binding proteins. Furthermore disclosed is the construction plan of such a bispecific molecule, which enables a person having ordinary skill in the art to readily construct such molecules. In certain embodiments, the molecular structure is thus a bispecific binding molecule, which exhibits superior anti-tumor activity in comparison to trastuzumab and pertuzumab and induces apoptosis in HER2-dependent cancer cells. This bispecific binding molecule can, in certain embodiments, be further modified by fusing moieties like e.g. toxins, half life extending groups and other functionalities.

The invention is exemplarily shown with bispecific binding molecules that are built of designed ankyrin repeat proteins (Binz et al. (2004) Nat. Bio. Tech. 22 575-582; US20120142611 (A1)-2012-06-07). However, there are no DARPin-specific functions in the molecules according to this disclosure, and thus the DARPins can be substituted by other binding proteins that serve to juxtapose the same epitopes such that they bring two HER2 molecules into a similar inactive orientation on the cell surface.

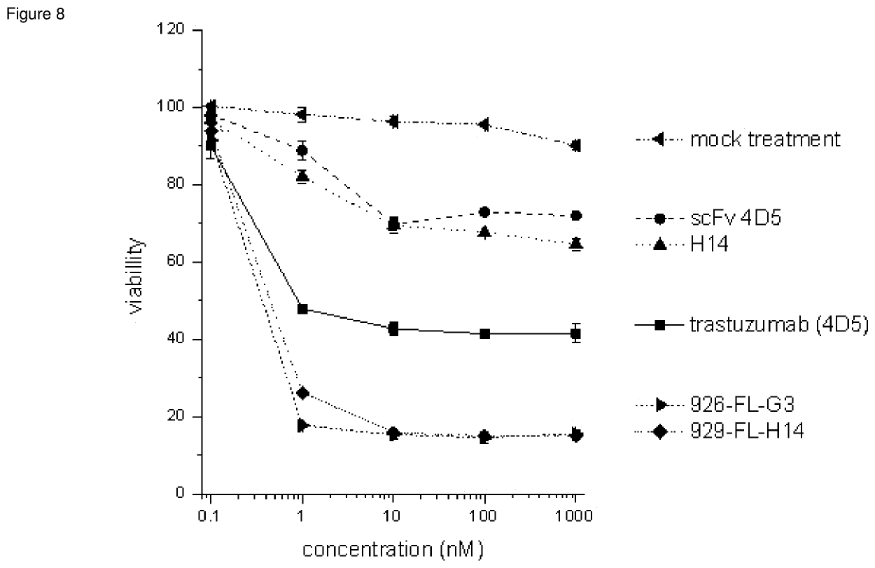

The agents and methods of the present invention are distinct from any method or reagent combination known in the art that binds to the same epitopes as the bispecific agent of the present invention. When converting IgGs into monovalent binding agents (by producing e.g. Fab fragments, or scFv fragments) the anti-tumor activity can vanish mostly or even completely. The results presented herein show that the scFv of 4D5 has only approx. 20% anti-tumor activity of the full length antibody in cell culture (measured in the absence of secondary functions like ADCC, FIG. 8).

Importantly, therefore, a bispecific agent comprising binding units that bind to the domain 1 of the ECD of HER2 and to domain 4 of the ECD of HER2 is not the sum of both modes of action that the respective antibody possesses, but is a new molecular entity according to the present invention.

Wherever alternatives for single separable features such as, for example, a first ligand, a second ligand, a bound epitope, a binding scaffold, a linker length or linker chemical constitution are laid out herein as “embodiments”, it is to be understood that such alternatives may be combined freely to form discrete embodiments of the invention disclosed herein. Thus, any of the alternative embodiments for a domain 1 epitope may be combined with any of the alternative embodiments of domain 4 epitope, and these combinations may be combined with any linker mentioned herein.

The invention is further illustrated by the following examples and figures, from which further embodiments and advantages can be drawn. These examples are meant to illustrate the invention but not to limit its scope.

Any U.S. patent or U.S. patent application cited in the present specification shall be incorporated herein by reference.

### EXAMPLES

### Example 1: Anti-Tumor Activity of the Bispecific Anti-HER2 Binding Agents in Comparison to Trastuzumab and Pertuzumab

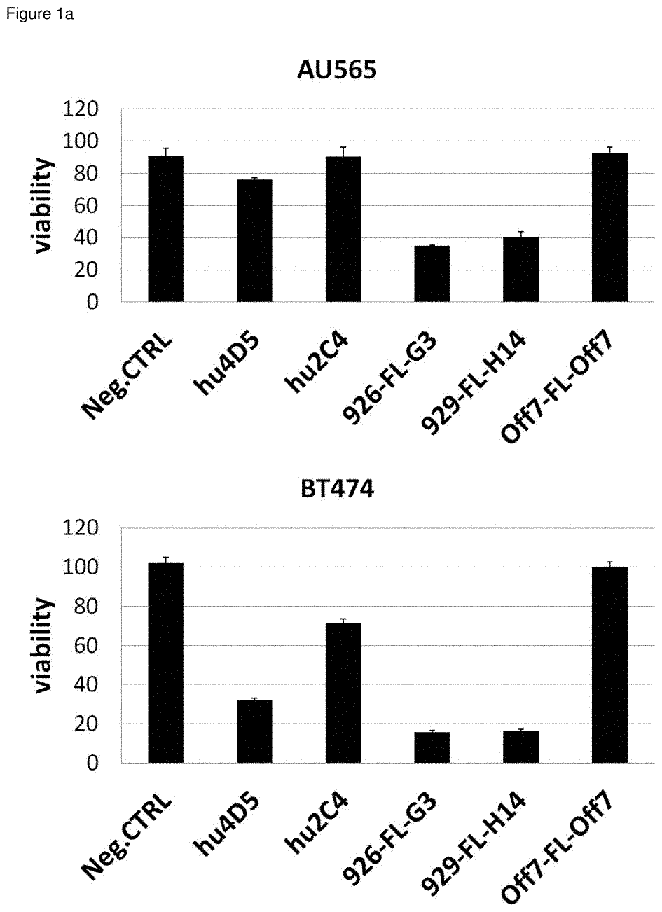

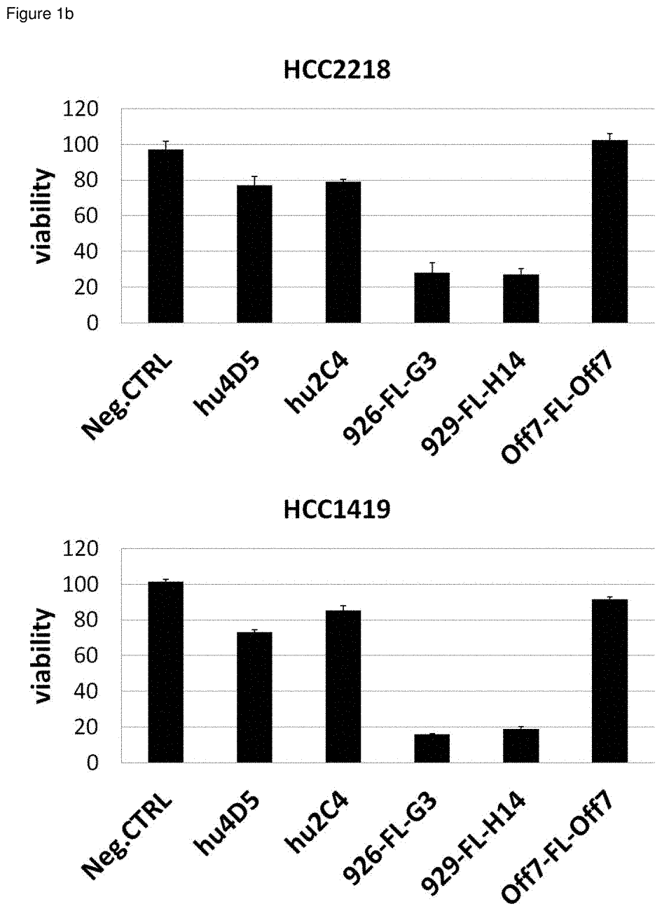

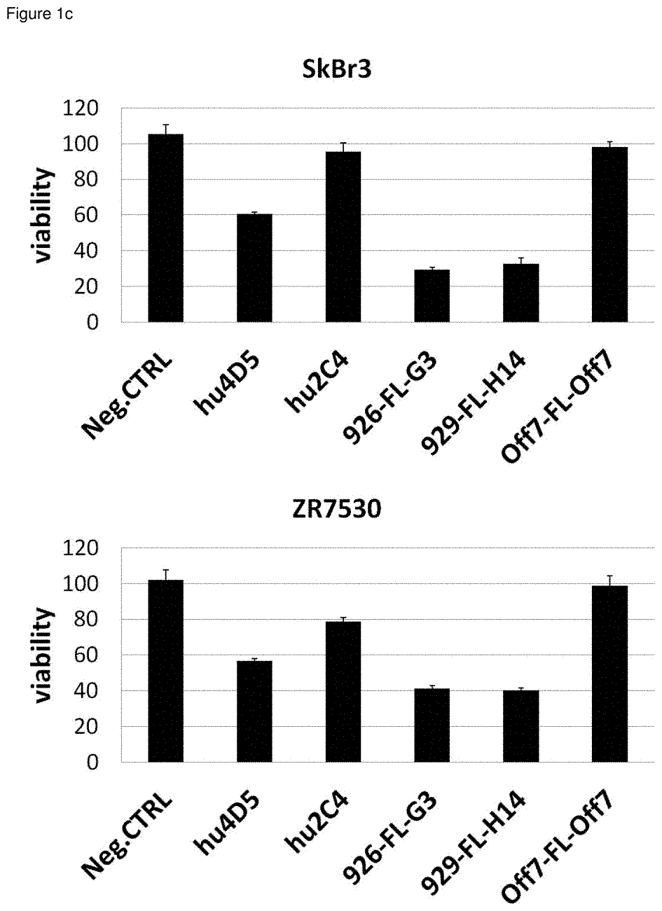

A XTT cell proliferation assay was performed with a panel of HER2 overexpressing cancer cell lines in 96-well tissue culture plates (FIG. 1). A defined number of cells were seeded in RPMI1640 medium containing 10% fetal calf serum (FCS). Cancer cells were treated for 4 days with 100 nM of anti-HER2 agents and controls. Measuring points were recorded in triplicates. XTT cell viability assays were developed according to the manufacturer's protocol. At a concentration of 100 nM, all anti-HER2 agents show maximal anti-tumor activity (titration not shown). The average of three data points is plotted with standard error. Data were normalized against the negative control on each plate, which corresponds to untreated cells (maximal growth). Bispecific targeting agents reduce cell growth of HER2-dependent cancer cells by 60-80%, while trastuzumab (hu4D5) reduces cell growth by only 20-60%. Bispecific targeting agents (926-FL-G3, 929-FL-H14) show consistently strong anti-tumor activity in all cell lines, while some cell lines show resistance against trastuzumab treatment. Sensitive cell lines can be roughly defined as HER2 dependent (e.g. HER2 overexpressing) and lacking any PI3K activating mutation.

FIG. 2 shows that bispecific targeting agents block entrance into S-phase and induce accumulation in G_{0/1}-Phase. BT474 cells were seeded 16 h before treatment in RPMI1640 containing 10% FCS. Anti-HER2 agents were added to a final concentration of 100 nM and cells were treated for 3 days. Afterwards, cells were fixed in 70% EtOH and stained with propidium iodide (PI). FACS measurements were gated to exclude cell debris in a forward vs. side scatter plot and 10⁴ events were recorded. PI fluorescence histograms were analyzed by FlowJo 7.2.5 software, and cell cycle distribution was fitted using the Dean-Jett-Fox algorithm, which excludes the apoptotic SubG1-population of cells. Treatment with bispecific targeting agents (926-FL-G3, 929-FL-H14) reduces S-phase and G_{2/M}-phase content in HER2-dependent cancer cells. It was shown that trastuzumab (hu4D5) treatment induces cell cycle arrest by blocking entrance into S-phase of sensitive HER2 dependent cancer cell lines. Here it is shown that bispecific targeting agents also induce cell cycle arrest in trastuzumab sensitive cell lines.

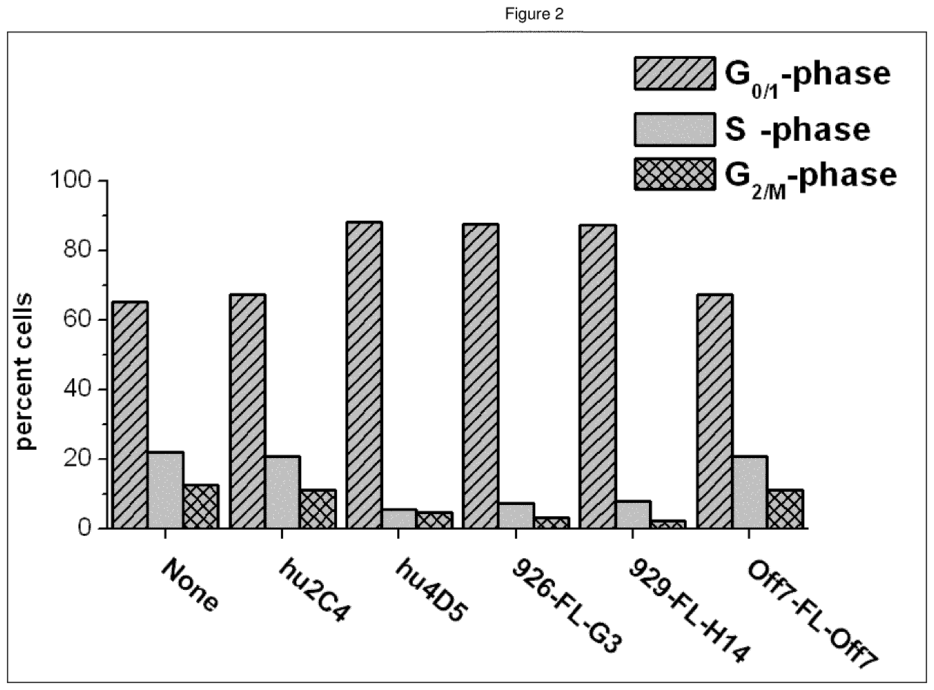

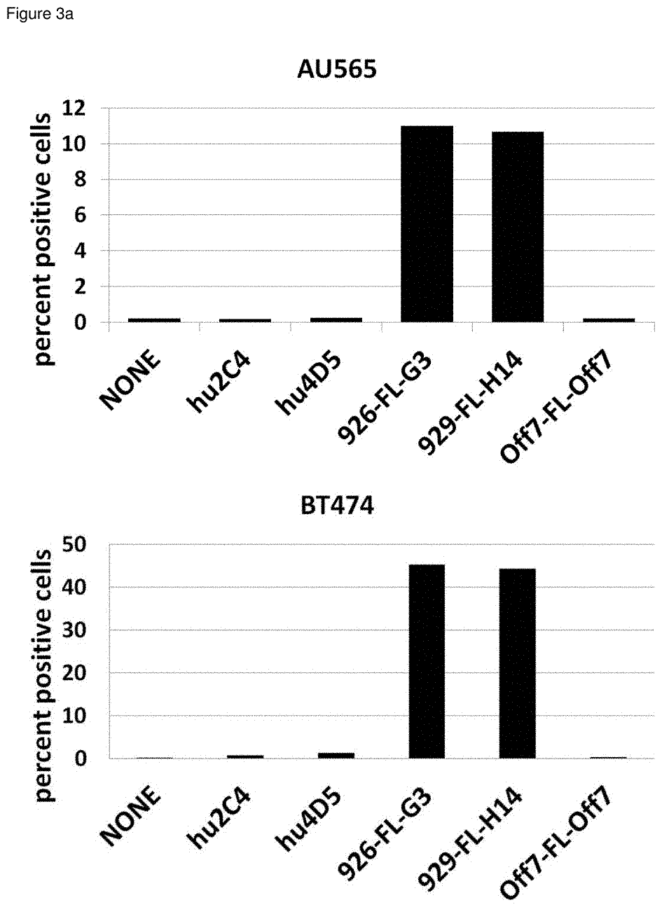

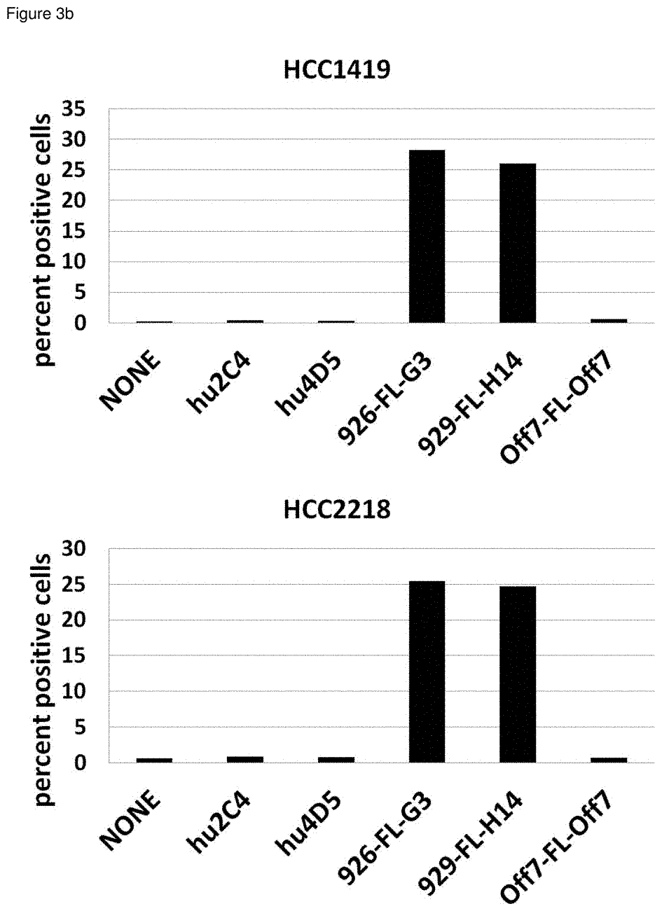

The terminal transferase dUTP nick end labeling (TUNEL) assay and quantification by flow cytometry was used to determine the portion of apoptotic cells upon treatment with anti-HER2 agents (FIG. 3). Cancer cells were seeded 16 h before treatment in RPMI1640 containing 10% FCS. Anti-HER2 agents (pertuzumab: hu2C4; trastuzumab: hu4D5; bispecific targeting agents: 926-FL-G3, 929-FL-H14; mock treatment: Off7-FL-Off7) were added to a final concentration of 100 nM and cells were treated for 3 days. Fractions of adherent and non-adherent cells were pooled. Cells were fixed in 2% paraformaldehyde, permeabilized in cold 0.1% sodium citrate containing 0.1% Triton X-100 for 2 min, washed three times with cold PBS and labeled with fluorescein-conjugated dUTP. FACS measurements were gated to exclude cell debris in a forward vs. side scatter plot and 10⁴ events were recorded. Measurements were plotted as an one parameter FL1 histogram plots (FITC fluorescence on the X-axis and counts on the Y-axis). Population of TUNEL positive (shift towards higher FL1) cells were quantified by one-dimensional regional gates which exclude TUNEL negative cells (auto fluorescence). Gates were applied according to negative control to exclude auto fluorescent cells. Treatment with bispecific targeting agents induces DNA degradation in HER2-dependent cancer cells, which is a hallmark of apoptosis. The number of TUNEL-positive cells correlates with the formation of a Sub-G₁ population, as determined by cell cycle analysis (data not shown). The quantification shows 30- to 80-fold higher TUNEL signals for the bispecific binding agents than for trastuzumab or pertuzumab in HER2-dependent cancer cells.

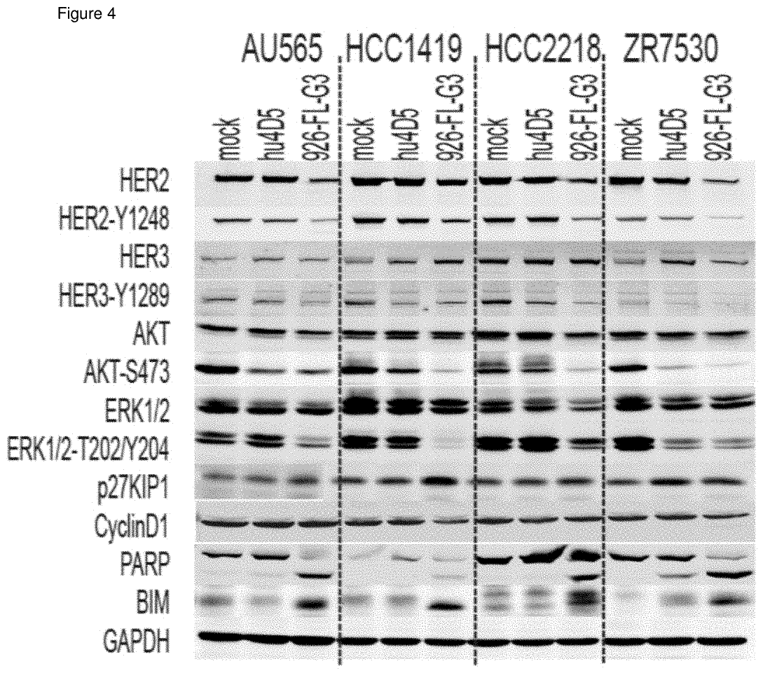

For Western blot analysis of the HER2/HER3 signalling pathway, PI3K/AKT and MAPK pathway and downstream targets of cell cycle and apoptosis, cancer cells were seeded 24 h before treatment in RPMI1640 containing 10% FCS. Anti-HER2 agents were added to a final concentration of 100 nM and cells were treated for 3 days. Afterwards, the fraction of detached apoptotic cells was collected and removed by centrifugation. Remaining attached cells were washed with cold PBS and scraped off into cold PBS_I (PBS containing protease inhibitors (Pefabloc, Leupeptin, Pepstatin, Marimastat) and phosphatase inhibitors (sodium orthovanadate, sodium metavanadate, sodium molybdate, β-glycerol phosphate, sodium fluoride)) on ice. Both cell fractions were pooled and washed in PBS_I. Afterwards, cells were lysed in PBS_I containing 1% Triton X-100 for 30 min at 4° C. on a rocker, and cell lysates were centrifuged at 20,000 g for 20 min at 4° C. Protein concentrations of the respective cell lysates were determined by BCA assays and samples were taken up in lithium dodecyl sulfate (LDS) loading buffer containing β-mercaptoethanol for complete reduction. Samples were heated for 5 min at 80° C. Samples were loaded on 10% SDS-PAGE and afterwards blotted on PVDF_FL membrane (Millipore) according to a BioRad protocol. After incubation with primary detection antibodies, western blots (FIG. 4.) were stained by secondary antibodies labeled with an infrared dye, and membranes were scanned on an Odyssey IR-fluorescence scanning system (LICOR). The following primary detection antibodies were used: Human Epidermal Growth Factor Receptor 2 (HER2); Phospho-Tyr 1248 Human Epidermal Growth Factor Receptor 2 (HER2-Y1248); Human Epidermal Growth Factor Receptor 3 (HER3); Phospho-Tyr 1289 Human Epidermal Growth Factor Receptor 3 (HER3-Y1289); Protein Kinase B (AKT); Phospho-Ser 473 Protein Kinase B (AKT-5473); p44/42 MAPK (ERK1/2); Phospho-Thr202/Tyr204 p44/42 MAPK (ERK1/2-T202/Y204); Cyclin-depended Kinase Inhibitor 1B (p27KIP1); CyclinD1 (CyclinD1); Poly ADP Ribose Polymerase (PARP); Bcl-2 Interacting Mediator of Cell Death (BIM); Glyceraldehyde 3-Phosphate Dehydrogenase (GAPDH).

For quantitative western blot analysis of the time course treatments, BT474 cells were seeded 24 h before treatment in RPMI1640 containing 10% FCS. Anti-HER2 agents were added to a concentration of 100 nM and cells were treated for 3 days. Afterwards, the fraction of only loosely adherent cells was washed away with cold PBS. Attached cells were scraped off in cold PBS_I (PBS containing protease inhibitors (Pefabloc, Leupeptin, Pepstatin, Marimastat) and phosphatase inhibitors (sodium orthovanadate, sodium metavanadate, sodium molybdate, β-glycerol phosphate, sodium fluoride)) on ice. Afterwards, cells were lysed in PBS_I containing 1% Triton X-100 for 30 min at 4° C. on a rocker and cell lysates were centrifuged at 20,000 g for 20 min at 4° C. Protein concentrations of the respective cell lysates were determined by BCA assays. HER2 receptor was immunoprecipitated by 901-FL-zHER2, a DARPin-affibody fusion construct, linked to Biosupport Ultra Link beads. HER2 receptor was depleted from BT474 cell lysate (corresponding to 1 mg protein in the lysate). Beads were washed three times with cold PBS_I. HER2 receptor was eluted from beads by heating to 80° C. for 5 min in LDS loading buffer containing β-mercaptoethanol for complete reduction. HER3 samples were heated for 5 min at 80° C. in LDS loading buffer containing β-mercaptoethanol for complete reduction. Samples were loaded on 10% SDS-PAGE and afterwards blotted on PVDF_FL membrane according to the BioRad protocol. Western blots were stained by secondary antibodies labeled with an infrared dye and membranes were scanned on an Odyssey IR-fluorescence scanning system (LICOR).

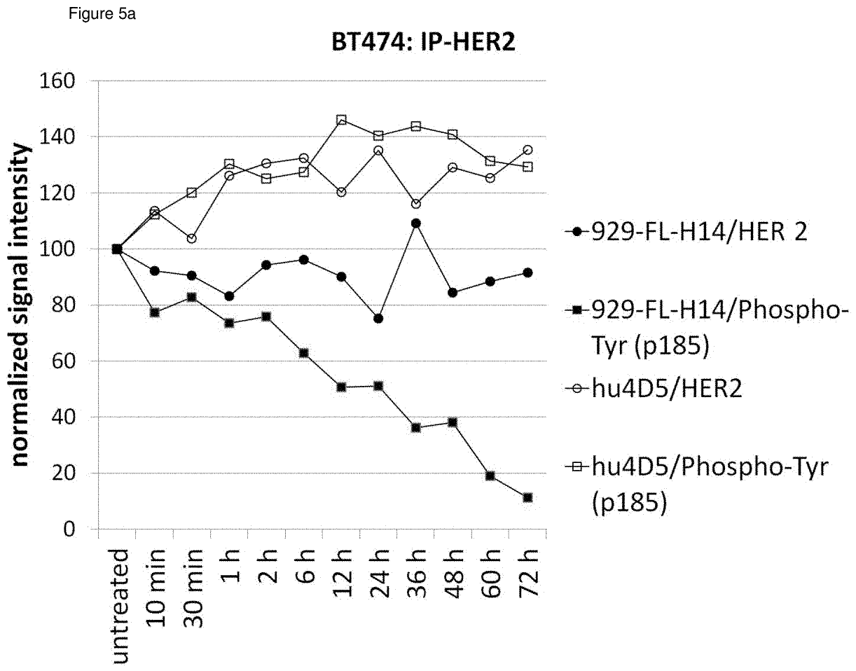

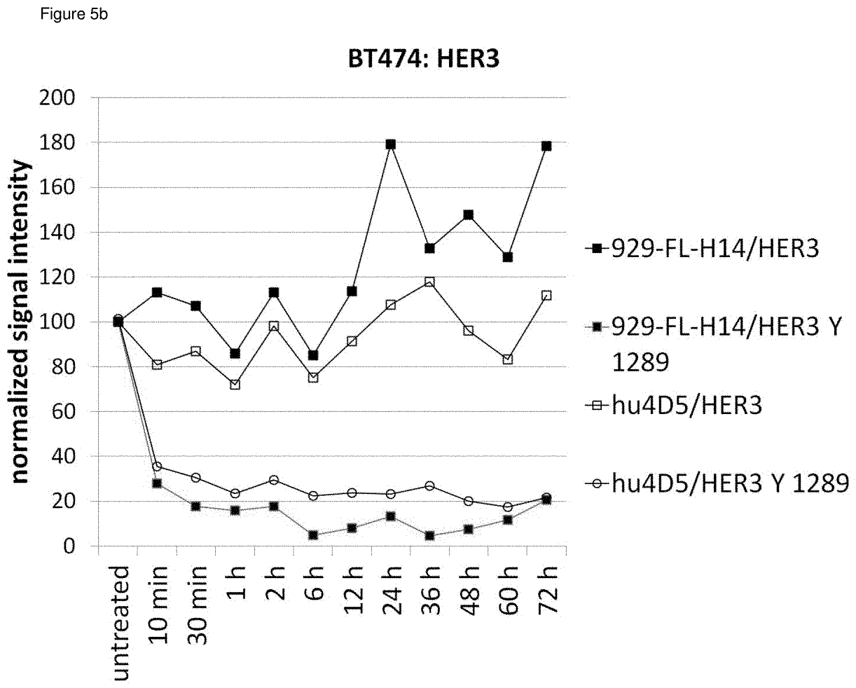

Bispecific agents down-regulate phospho-HER2 levels consistently in all HER2-dependent cancer cells. Down-regulation of phospho-HER2 can correlate with down-regulation of HER2 expression level, which was observed in the fractions of apoptotic cells (FIG. 4). Constant HER2 expression levels were observed in the fraction of attached cells for e.g. BT474 and SkBr3, while phospho-HER2 levels were strongly reduced (FIG. 5). Therefore, down-regulation of HER2 expression can be observed in the apoptotic fraction of HER2-dependent cancer cells but is probably not the cause for induction of apoptosis. Rather, down-regulation of phosho-HER2 simultaneously with reduction of phosho-HER3 is the cause for induction of apoptosis. Down-regulation of phospho-HER3 can be observed after treatment with bispecific targeting agents and trastuzumab. Bispecific targeting agents show stronger down-regulation of phospho-HER3 than trastuzumab. Up-regulation of HER3 expression can be observed after treatment with bispecific targeting agents. A feedback loop sensing inhibition of phospho-AKT and, consequently, up-regulation of HER3 expression has been proposed. Bispecific targeting agents reduce phospho-AKT (downstream HER3) and phospho-ERK (downstream HER2) signaling simultaneously. Trastuzumab treatment mainly down-regulates phospho-AKT, while in ZR7530 cells, trastuzumab treatment leads to a down regulation of phospho-ERK. Cell cycle regulators p27KIP1 (inhibitor of cyclin-dependent kinases) is up-regulated and CyclinD1, which mediates G1/S-phase transition, is down-regulated in several HER2-dependent cancer cell lines. Again, inhibition of the cell cycle is not necessarily observed by bispecific targeting agents, but cell cycle arrest is observed in cell lines which are sensitive to trastuzumab treatment. BIMₛ up-regulation and PARP cleavage (up-regulation of PARP p89) is observed in all HER2-dependent cancer cell lines, which show induction of apoptosis after treatment with bispecific targeting agents. ZR7530 and BT474 cells show also PARP cleavage after treatment with trastuzumab, but bispecific targeting agents show consistently stronger signals.

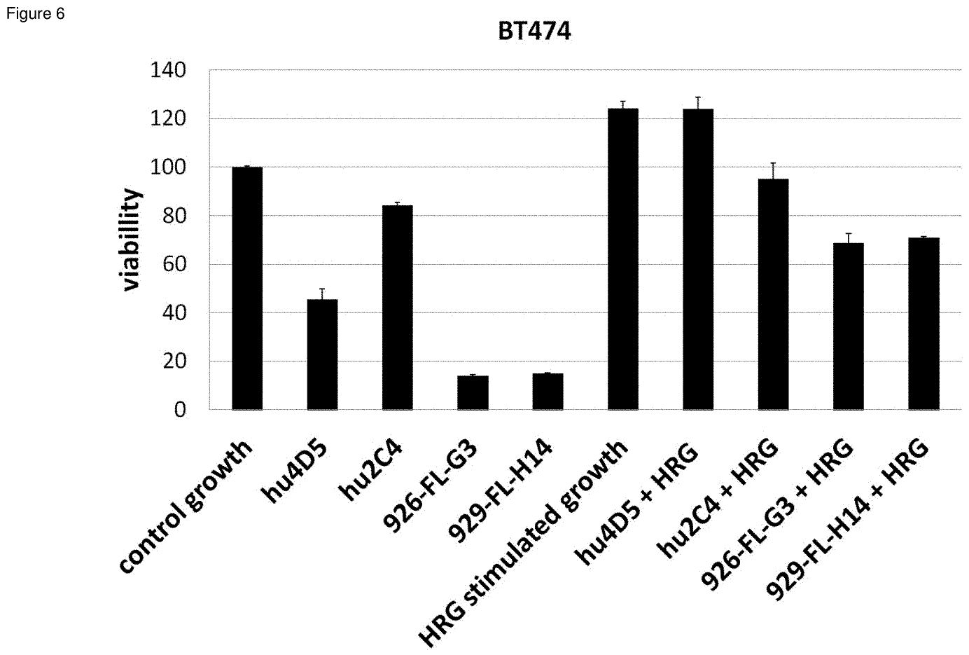

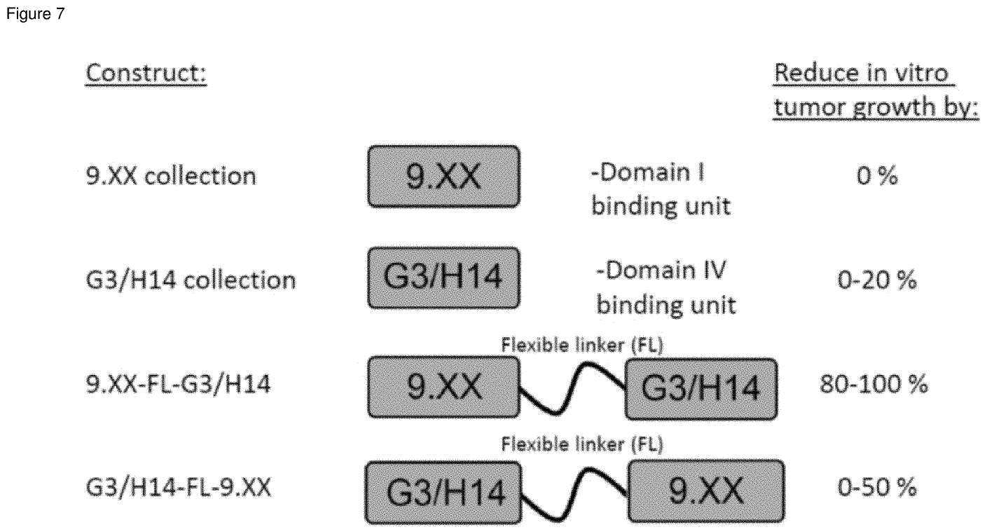

XTT cell proliferation assays were performed with BT474 cells in 96 well tissue culture plates (FIG. 6). Cells were seeded at a density of 10⁴ cells/cm² 16 hours before treatment in RPMI1640 containing 1% FCS (low concentration of additional growth factors). Cells were pre-treated with 100 nM anti-HER2 agents for 2 hours. Afterwards, cells were stimulated by adding heregulin beta-1 (HRG) to a concentration of 1 nM (Recombinant Human NRG1-β1/HRG1-β1: 26.9 kDa). HRG treatment leads to an increase of viable BT474 cells by 20%, compared to the control growth in 1% FCS alone (FIG. 6). The single treatments with anti-HER2 agents are thus compared to the corresponding controls in the absence (100% viability) or presence (120% viability) of HRG. Trastuzumab (hu4D5) treatment reduced viability by 50-60% in the absence of HRG, but did not show anti-tumor activity in ligand-stimulated cancer cells. Trastuzumab completely looses anti-tumor activity in presence of 1 nM HRG. Pertuzumab (hu2C4) treatment reduced viability by 20-30% in the presence or absence of HRG. Bispecific targeting agents reduced the viability by 80-90% in the absence of HRG and also showed 40-50% reduction in the presence of HRG. Therefore, the bispecific targeting agents show strongest anti-tumor activity both in the presence and in the absence of HRG. The additive effect of trastuzumab and pertuzumab resembles the individual maximal anti-tumor activity of the single agents (data not shown), but has no significant mechanistically synergism in in-vitro models. Therefore, the mechanism of action of bispecific targeting agents is superior to the treatment with trastuzumab combined with pertuzumab in in-vitro models. The treatment with the bispecific reagents exceeds the effect of the sum of effects from both antibodies.

A person skilled in the art will appreciate that the XTT-assay is a suitable test for the determination of the cytotoxicity and for the evaluation of the potential of anti-tumor candidate compounds (see Jost et al, (1992) Journal of Immunological Method, 147, 153-165; Scudiero et al. (1988) Cancer Research, 48, 4827-4833; Andjilani et al, (2005) Int. J. Cancer, 117, 68-81; Rubinstein et al. (1990) J Natl Cancer Inst, 82(13), 1113-1117; Monks et al. (1991) J Natl Cancer Inst, 83(11), 757-766).

### Example 2: Construction Plan of Bispecific Anti-HER2 Targeting Agents that Induce Apoptosis in HER2 Dependent Cancer Cells

Generation of Binding Agents that Form the Components of the Active Molecule

Binding molecules were obtained by ribosome display selection of ankyrin repeat protein libraries for specific binding to the full length extracellular domain of HER2 (ECD HER2) by methods previously disclosed (Zahnd et al. (2006) J. Biol. Chem. 279, 18870-18877).

Preparation of the Biotinylated HER2 Target

In order to obtain binders to the individual domains, the different individual domains of HER2 were individually expressed in insect cells, using a baculovirus expression system. Thereby, it is guaranteed that binders selected will be directed towards the domain of interest. Briefly, recombinant ErbB2-ectodomains carrying an N-terminal melittin signal sequence (MKFLVNVALVFMVVYISYIYA, SEQ ID 101) and an N-terminal His6 tag were expressed in Spodoptera frugiperda (Sf9) cells using baculoviral vectors. Sf9 cells were grown to a density of 4×10⁶ cells/mL and co-infected with the respective virus at a MOI of 1. 72 h post-infection, cells were harvested by centrifugation (30 min, 5,000 g, 4° C.) and the cleared medium was subjected to immobilized metal ion affinity chromatography (IMAC) purification with Ni-NTA Superflow purification resin.

To generate binders against any domain of the extracellular region, the extracellular domain (residues 1-621) of HER2 was used as target for the selection with ribosome display (Zahnd et al., J. Biol. Chem. (2006) 281: 35167-35175) or, to generate binders against the first three domains, HER2 residues 1-509 was used.

For immobilization, aliquots of these target proteins (200-600 μg) were chemically biotinylated using EZ-Link Sulfo-NHS-SS-Biotin. Due to the size difference of the target proteins, a variable molar excess of the biotinylating reagent relative to the target protein was used (6-fold for HER2 1-621 or 1-509, 3-fold for the single domains). Reaction conditions were used according to the supplier's manual. Successful biotinylation was confirmed by ELISA and Western blot experiments. The biotinylated HER2 constructs were dialyzed extensively against PBS150.

Target proteins had to be immobilized for selection. To avoid partial protein denaturation of the target proteins that may result from direct immobilization on solid plastic (i.e. polystyrene) surfaces, biotinylated target proteins were bound to neutravidin or streptavidin, which had been immobilized directly on a solid plastic surface, as follows: neutravidin (66 nM, 100 μl/well) or streptavidin (66 nM, 100 μl/well) in PBS was immobilized on MaxiSorp plates (Nunc, Denmark) by incubation at 4° C. overnight. The wells were blocked with 300 μl of PBSTB (PBS containing 0.1% Tween-20, 0.2% BSA) for 1 h at room temperature. Binding of the biotinylated target proteins (100 μl, 100 nM for selection) in PBSTB was allowed to occur for 1 h at 4° C. For the first selection round on immobilized target protein, requiring larger volumes, neutravidin (66 nM, 4 ml/tube) in PBS was immobilized on MaxiSorp Immunotubes by incubation at 4° C. overnight. The tubes were blocked with 4 ml of PBSTB for 1 h at room temperature. Binding of the biotinylated target proteins (4 ml, 100 nM) in PBSTB was allowed to occur for 1 h at 4° C. For selection on immobilized target protein, neutravidin and streptavidin were used alternately in selection rounds to avoid selection of binders against these proteins.

Ribosome Display

Ribosome display followed the published protocols (Dreier et al. (2012) Methods Mol. Biol.

805, 261-286; Zahnd et al. (2007) Nat. Methods 4, 269-279.) Typically 3 or 4 rounds were carried out. The first round was always carried out on plates, the later rounds in some of the selection on plates, in others in solution, where the biotinylated HER2 target is then bound to streptavidin-coated magnetic beads, as described in the protocols in detail (Dreier et al. (2012) Methods Mol. Biol. 805, 261-286; Zahnd et al. (2007) Nat. Methods 4, 269-279.).

In the forth round, the selection pressure was increased by applying off-rate selection. For this purpose, after the in vitro translation was stopped by 5-fold dilution into ice-cold WBT buffer (50 mM Tris acetate, pH 7.5, 150 mM NaCl, 50 mM Mg(CH3COO⁻)₂, 0.05% Tween 20), biotinylated HER2 construct was added to a final concentration of 10 nM, and the translation was allowed to equilibrate for 2 h at 4° C. The translation reaction was split into two aliquots, and non-biotinylated HER2 construct was added to a final concentration of 1 μM to each aliquot, corresponding to a 100-fold excess over biotinylated antigen. The aliquots were incubated for 2 and 20 h, respectively, to increase the selection stringency for slower off rates. Ribosomal complexes were recovered using 30 μl of streptavidin-coated magnetic beads. In a subsequent round, 175 nM biotinylated HER2 construct was immobilized on a NeutrAvidin-coated Maxisorp plate, i.e. rather non-stringent conditions to collect the binder (“collection round”) (Dreier et al. (2012) Methods Mol. Biol. 805, 261-286; Zahnd et al. (2007) Nat. Methods 4, 269-279.)

In all selection rounds on solid-phase immobilized HER2 construct, a prepanning step of 30 min on a neutravidin-coated Maxisorp plate was performed as described (Dreier et al. (2012) Methods Mol. Biol. 805, 261-286; Zahnd et al. (2007) Nat. Methods 4, 269-279.). After prepanning, the translation extracts were allowed to bind for 45 min to HER2 construct-coated Maxisorp plates. Retained complexes were extensively washed with WBT buffer.

Phage Display

Phage display of the DARPin library followed the published protocol (Steiner et al. (2008) J. Mol. Biol. 382, 1211-1227). The immobilization of the various biotinylated HER2 constructs has been described above.

Unless stated otherwise, all steps of the phage display selection were carried out at room temperature. Selection rounds were performed either on biotinylated target protein in solution with subsequent capturing on streptavidin-coated magnetic beads (referred to as: “target protein in solution”) or on biotinylated target protein bound to neutravidin or streptavidin, which had been directly immobilized on a solid plastic surface (referred to as: “immobilized target protein”), as described below. Very good results were obtained when performing the first selection round of selection on immobilized target protein, presumably because of the greater efficiency of capturing binders (especially important in the first round), followed by further rounds on target protein in solution, presumably because of the lower enrichment of background binders

Selection on Target Proteins in Solution

When the first selection cycle was done in solution, about 2.5×10¹³ phage particles of the phage DARPin library were incubated for 1 hour with 100 nM biotinylated target protein in 2 ml PBSTB for the first round of selection. In subsequent selection rounds, about 10¹² phage particles were used (see below). The phage-antigen complexes were then captured on 100 μl streptavidin-coated paramagnetic beads (10 mg/ml) for 20 min. After washing the beads eight times with PBST (PBS, 0.1% Tween-20) the phage particles were eluted with 200 μl of 100 mM triethylamine (Et3N, pH not adjusted) for 6 min, followed by 200 μl of 100 mM glycine-HCl, pH 2, for 10 min. Eluates were neutralized with 100 μl of 1 M Tris-HCl, pH 7, or 18 μl of 2 M Tris-base, respectively, combined and used to infect 5 ml of exponentially growing E. coli XL1-Blue cells. After shaking for 1 hour at 37° C., cells were expanded into 50 ml of fresh 2YT medium (5 g NaCl, 10 g yeast extract, 16 g tryptone per liter) containing 10 μg/ml cam and incubated at 37° C. with shaking. After a maximum of 5 h (shorter times if OD₆₀₀=0.5 was reached earlier), isopropyl-β-D-thiogalactoside (IPTG) was added to a final concentration of 0.2 mM and 15 minutes later the phage library was rescued by infection with VCS M13 helper phage at 10¹⁰ pfu (plaque forming units) per ml (multiplicity of infection 20). Cells were grown overnight at 37° C. without the addition of kanamycin. Cells were removed by centrifugation (5600 g, 4° C., 10 min) and 40 ml of the culture supernatant was incubated on ice for 1 hour with one-fourth volume of ice-cold PEG/NaCl solution (20% polyethylene glycol (PEG) 6000, 2.5 M NaCl). The precipitated phage particles were then collected by centrifugation (5600 g, 4° C., 15 min) and redissolved in 2 ml of PBS and used for the second round of selection.

For the subsequent selection rounds, about 10¹² of the amplified phage particles were used as input and incubated with 100 μl of streptavidin-coated paramagnetic beads for 1 h to remove unspecific and streptavidin binding phage particles. After removing the beads, phage particles were incubated for 1 hour with 100 nM biotinylated target protein, complexes were captured on fresh beads, beads were washed 12 times with PBST, phages eluted with 400 μl of 100 mM glycine-HCl, pH 2, for 10 min, the eluate neutralized with 36 μl of 2 M Tris-base and phage particles amplified and purified as described above. After three rounds, enrichment of phage particles displaying DARPins binding specifically to the HER2 target construct was monitored by phage ELISA. About 5×10¹⁰ phage particles (estimated spectrophotometrically) of the initial library and the amplified pools of each selection round were pipetted to wells with and without immobilized target protein and incubated at RT for 2 h. After washing the wells four times with 300 μl of PBST, bound phage particles were detected with mouse anti-M13 antibody horseradish peroxidase conjugate and soluble BM Blue peroxidase (POD) substrate.

Selection on Immobilized Target Proteins

For the first selection cycle about 3.5×10¹³ phage particles of the phage DARPin library were added to an immunotube containing the immobilized target protein (biotinylated target protein bound to neutravidin, which had been directly immobilized on the solid plastic surface) and incubated with rotation for 2 h. After rinsing the tube ten times with PBST, the phage particles were eluted with 500 μl of 100 mM Et₃N (pH not adjusted) for 6 min, followed by 500 μl of 100 mM glycine-HCl, pH 2, for 10 min. Eluates were neutralized with 250 μl of 1 M Tris-HCl, pH 7, or 45 μl of 2 M Tris-base, respectively, combined and used to infect 13 ml of exponentially growing E. coli XL1-Blue cells. After shaking for 1 hour at 37° C. cells were expanded into 130 ml of fresh 2YT medium containing 10 μg/ml chloramphenicol (cam) and incubated at 37° C. with shaking. Phage amplification and precipitation was done as described above.

In the subsequent selection rounds about 10¹² of the amplified phage particles were first incubated in a blocked immunotube (coated either with neutravidin or streptavidin used for immobilization of the target protein in the previous round of selection and BSA) one hour to remove neutravidin, streptavidin or unspecific binding phage particles. For the binding selection the phage particles were incubated for one hour in four wells containing the immobilized biotinylated target protein (directly coated neutravidin or streptavidin were alternately used in subsequent selection rounds). The wells were washed 12 times with PBST, phages eluted from each well with 100 μl of 100 mM glycine-HCl, pH 2, for 10 min, the combined eluates neutralized with 36 μl of 2 M Tris-base and phage particles amplified and purified as described above. After three rounds, enrichment was determined by phage ELISA as described above.

Phage Display from Antibody Library

Single-chain antibody fragments (scFv) were selected for binding to HER2, which have a molecular weight of 30 kDa, from HuCAL-1 (Knappik et al., 2000), a library of synthetic human antibody fragments. The library has a diversity of about 2×10⁹ members (Knappik et al., JMB, 2000, 296(1), 57-86). M13 phages presenting the HuCAL-1 scFv library as a fusion to the CT domain of g3p coat protein were selected for binding to soluble biotinylated HER2 domain 1 or domain 4, which was immobilized on neutravidin or streptavidin on microtiter wells as described above.

Phage selections were performed by incubating 50 pmol of biotinylated antigen with 1 pmol of phages in 100 μl PBS 0.5% BSA for 1 h at 4° C. The complexes were captured with 1 mg of BSA-blocked streptavidin magnetic particles and washed 10 times with PBS 0.5% BSA. Bound phages were eluted with 100 mM glycine, pH 2.2, and neutralized with the same volume of 1 M Tris, pH 8. E. coli TG1 cells were infected with eluted phages and plated on LB agar plates containing 1% glucose and 34 mg/l chloramphenicol. The plates were incubated overnight at 30° C., and bacteria were scraped off to inoculate 2×YT medium containing 1% glucose and 34 mg/l chloramphenicol. The culture was incubated at 37° C. and at OD₆₀₀=0.5 the phage library was rescued by infection with VCS M13 helper phage (Stratagene). The bacteria were harvested by centrifugation and resuspended in 2×YT medium containing 30 mg/l kanamycin, 34 mg/l chloramphenicol, 0.1 mM IPTG and grown overnight at 30° C. Phages were precipitated from the culture supernatant by addition of polyethylene glycol PEG-6000 (3.3% final concentration), NaCl (0.4 M final concentration). Phages were resuspended in H₂O, precipitated by addition of polyethylene glycol PEG-6000 (3.3% final concentration), NaCl (0.4 M final concentration) and resuspended in PBS.

After the fourth and fifth round of phage display, pools of selected scFv-encoding sequences were subcloned via restriction sites XbaI and EcoRI into the expression plasmid pMX7 (Knappik et al., JMB, 2000, 296(1), 57-86). E. coli SB536 cells were transformed with the constructed vector. Bacteria were grown at 37° C. in 2×YT medium containing 0.1% glucose and 34 mg/l chloramphenicol. At OD₆₀₀=0.5 cultures were induced with 1 mM IPTG. ScFv fragments are secreted to the periplasm of E. coli. For small-scale expressions, cultures were incubated for 5 h after induction at 30° C. For periplasmic extracts, cells were collected by centrifugation and incubated overnight in 300 mM boric acid, 150 mM NaCl, 2 mM EDTA, pH 8, at 4° C. After centrifugation, the supernatant was used for enzyme linked immuno-sorbent assay (ELISA) screening.

For large-scale expression of scFv fragments, cultures were incubated for 20 h at 22° C. Bacteria were collected by centrifugation and resuspended in 50 mM NaH₂PO₄, 300 mM NaCl, pH 8. After addition of a spatula tip of DNAseI and 2 mM MgCl₂, bacteria were lysed in a French pressure cell. The lysate was filtered and purified on Ni-NTA agarose, washing with 16 column volumes of 50 mM NaH₂PO₄, 300 mM NaCl, pH 8; 12 column volumes of 50 mM NaH₂PO₄, 900 mM NaCl, pH 8; 16 column volumes of 50 mM NaH₂PO₄, 300 mM NaCl, 0.1% Triton X-100, pH 8; and 8 column volumes of 50 mM NaH₂PO₄, 300 mM NaCl, pH 8. Eluates were concentrated by ultra-centrifugation and buffer-exchanged to PBS using Micro BioSpin P-6 columns. For proliferation assays, samples were additionally purified on Detoxi-Gel endotoxin removal columns and eluted with PBS. When stored at 4° C. under sterile conditions, purified scFv fragments maintained unchanged binding activity for more than 3 months.

Bispecific scFv1-Linker-scFv2 Constructs

Antibody scFv fragments binding to either HER2 domain 1 or HER2 domain 4 were identified by ELISA as described above. From these scFv fragments, a series of bispecific scFv1-linker-scFv2 constructs (bispecific tandem scFv), where always a HER2 domain 1 binder was connected to a HER2 domain 4 binder (in either orientation), was constructed as follows: Since all HuCAL scFv fragments have common internal restriction sites, a vector could be constructed, pHu202, in which the upstream scFv fragment is connected via a flexible linker to the downstream fragment, which does not have a signal sequence, resulting in the arrangement phoA-scFv1-linker-scFv2, where phoA is the secretion signal. The linker segment can be exchanged via unique restriction sites that have been engineered into this fragment at its flanks, NotI and SfiI. Thus, all combinations of potential active bispecific antibodies were conveniently constructed by ligating the linker-scFv2 unit into the secretion vector containing phoA-scFv1, downstream of scFv1. After the active combinations had been identified, the linker was systematically varied in these constructs, by exchanging it into a series of linkers with different length, ligating it via NotI and SfiI. For large-scale expression of the scFv1-linker-scFv2 fragments, cultures were incubated for 20 h at 22° C. Bacteria were collected by centrifugation and resuspended in 50 mM NaH₂PO₄, 300 mM NaCl, pH 8. After addition of a spatula tip of DNAseI and 2 mM MgCl₂, bacteria were lysed in a French pressure cell. The lysate was filtered and purified on Ni-NTA agarose, washing with 16 column volumes of 50 mM NaH₂PO₄, 300 mM NaCl, pH 8; 12 column volumes of 50 mM NaH₂PO₄, 900 mM NaCl, pH 8; 16 column volumes of 50 mM NaH₂PO₄, 300 mM NaCl, 0.1% Triton X-100, pH 8; and 8 column volumes of 50 mM NaH₂PO₄, 300 mM NaCl, pH 8. Eluates were concentrated by ultra-centrifugation and buffer-exchanged to PBS using Micro BioSpin P-6 columns. For proliferation assays, samples were additionally purified on Detoxi-Gel endotoxin removal columns and eluted with PBS.

Bispecific Diabodies

The cloning of the bispecific diabodies is similar to that of tandem scFvs, but with some important differences. We needed to clone two genes, phoA-VH1-VL2, followed by phoA-VH2-VL1. For simplicity, we opted for two promoters, each driving one of the genes. VH1 and VL1 are the heavy and light chain variable regions of svFv1, and VH2 and VL2 correspondingly of svFv2, but in the diabody arrangement they are now connected to the partner chain of the other scFv. The modularity of the synthetic HuCAL library with its conserved restriction sites within the synthetic genes makes this cloning very convenient. As can be seen, it was only necessary to exchange VH (or VL) between to scFv fragments, using the unique restriction sites by which VH and VL are flanked in the scFv fragment (Knappik et al., 2000). The whole cassette, promoter-phoA-VH1-linker-VH2 had been flanked by Not1 and SfiI sites in the newly created vectors pDia202, while in pDia203, the same sites had been engineered downstream of the scFv expression cassette. Thus, the complete unit promoter-phoA-VH1-linker-VH2 could be cloned into a vector already containing promoter-phoA-VH2-linker-VH1. Thus, both chains of the diabody were encoded on the same plasmid. Both are secreted to the periplasm where they assemble. For large-scale expression of the diabodies, cultures were incubated for 20 h at 22° C. Bacteria were collected by centrifugation and resuspended in 50 mM NaH₂PO₄, 300 mM NaCl, pH 8. After addition of a spatula tip of DNAseI and 2 mM MgCl₂, bacteria were lysed in a French pressure cell. The lysate was filtered and purified on Ni-NTA agarose, washing with 16 column volumes of 50 mM NaH₂PO₄, 300 mM NaCl, pH 8; 12 column volumes of 50 mM NaH₂PO₄, 900 mM NaCl, pH 8; 16 column volumes of 50 mM NaH₂PO₄, 300 mM NaCl, 0.1% Triton X-100, pH 8; and 8 column volumes of 50 mM NaH₂PO₄, 300 mM NaCl, pH 8. Eluates were concentrated by ultra-centrifugation and buffer-exchanged to PBS using Micro BioSpin P-6 columns. For proliferation assays, samples were additionally purified on Detoxi-Gel endotoxin removal columns and eluted with PBS.

In addition, single-chain diabody constructs were constructed as described in Example 5, (analogous to constructs described by Völkel et al. (2001), Protein Engineering 14, 815-823).

Analysis of Single Binding Agents

Binding agents were characterized by means of enzyme-linked immunosorbent assay (ELISA). ELISAs, using the full length extracellular domain of HER2 (ECD HER2) for coating, were carried out to show binding of all individual binding agents. ELISA, using a truncated form of ECD HER2 (domain 1-3) as target, were performed to show specific binding of the DARPins to this part of HER2 ECD. This was originally applied to the collection of the 9XX series of binders (molecules originating from the HER2_509 selection). Domain 4 binders G3 and H14 were identified by binding to full length ECD HER2 but an absence of binding to the truncated ECD HER2 comprising only domains 1 to 3.

Specific binding experiments were carried out on the surface of viable HER2 overexpressing cancer cells e.g. BT474, SkBr3, SkOv3, using standard flow cytometry methods. Multiple fluorescent detection systems, like e.g. detection of the His-Tag by an anti His-tag antibody, followed by a secondary antibody labeled with Alexa488, or alternatively, genetic superfolder GFP (sfGFP) fusions with the binding molecules or using directly Alexa488-labeled binding reagents, were used to confirm specific binding of all single binding reagents to the surface of HER2 overexpressing cancer cells. The binding to a single epitope was confirmed by the analysis of mean fluorescence intensities, resulting in similar values for all binders at saturation, and more importantly, by complete inhibition of the signal when competed to an unlabeled control binding to said epitope. The single binding reagents also passed different quality control measurements like e.g. size exclusion chromatography, multi-angle light scattering and polyacrylamide gel electrophoresis (PAGE).

Competitive Binding Analysis of Binding Reagents

Competitive binding analysis was performed to characterize the epitopes of the binding agents of the 9XX collection. All binding agents of the 9XX collection compete for binding to a similar epitope on domain 1 of HER2, except binder 9.01. Competitive binding FACS analysis was also performed with domain 4 binding agents versus trastuzumab. Groups of competing and non-competing binding agents were identified. Importantly, binding to the trastuzumab epitope is not a prerequisite for the anti-tumor activity of the bispecific molecules (G3 does not compete with trastuzumab for binding). Binder H14 does compete with G3 and does show competition with trastuzumab.

Competitive binding FACS analysis performed with the 9XX binding molecules versus pertuzumab binding did not show competition. None of the single binding agents binds to the pertuzumab epitope. ELISA, using the domain 1 of the ECD HER2 as target, was performed to show specific binding of the 9XX collection.

Table 1 summarizes properties of preferential binding units (that can be components of bispecific molecules with bioactivity) and control binding units (which do not contribute bioactivity) for the construction of bispecific binding agents with superior anti-tumor activity. Listed are the single domains of the extracellular part of HER2 that are bound by the single agents. The epitope is characterized by inhibition of a binding assay performed in ELISA or on the surface of HER2 overexpressing cancer cells by means of flow cytometry. Crystal structure data are available for the indicated binding agents, which characterize the specific epitopes in detail on the single amino acid level. For the construction of potent bispecific anti-tumor agents, a binding agent which targets domain 1 of HER2 is preferentially fused to a binding agent that targets domain 4 of HER2 from the list of indicated binding agents.

TABLE 1
Summary of single binding agents

| | Binds to | | | Strong anti-tumor |
|---|---|---|---|---|
| | HER2 | Competitive Binding to | Crystal Structure | activity in |
| | domain: | HER2 known with: | available: | bispecific setup: |
| G3 | IV | H14 | YES | YES |
| H14 | IV | G3; 4D5 | — | YES |
| 902 | I | 929; 926 | — | YES |
| 903 | I | 929; 926 | — | YES |
| 910 | I | 929; 926 | — | YES |
| 916 | I | 929; 926 | — | YES |
| 926 | I | 929; 926 | YES | YES |
| 929 | I | 929; 926 | YES | YES |
| 930 | I | 929; 926 | — | YES |
| H01 | I | 929; 926 | — | YES |
| H03 | I | 929; 926 | — | YES |
| Off7 | none | none | YES | — |
| 4D5, trastuzumab | IV | H14, Nanobody, Zybody | YES | YES |
| 2C4, pertuzumab | II | Nanobody, Zybody | YES | — |
| zHER2 | III | none | YES | — |
| A21 | I | none | YES | YES |

The domain 1-binding scFv A21 is described in example 5.

Expression of Bispecific Binding Agents

The genes or coding sequences of the bispecific molecules were constructed in a vector pQiBi-01- (or -11-; -12-; -22-; -23-; -33-); using conventional restriction digest and ligation techniques with a BamHI/HindIII restriction site for the N-terminal binding molecules and BglII/BsaI restriction sites for the C-terminal binding molecules. This vector is derived from pQE30, but encodes the lacl^{q} gene and unique restriction sites (BamHI/HindIII and BglII/BsaI, respectively) to clone one binder upstream, the other downstream of a linker via BamHI/HindIII. The numbers indicate the different linker lengths, where each unit is a (Gly₄Ser) unit. E.g., the pQiBi-22-vector encodes 4 (Gly₄Ser) units between the binders. Bispecific constructs were expressed in E. coli strains XL1blue or E. coli BL21 using the lac-operon induction system by isopropyl-β-D-thiogalactopyranoside (IPTG). Bacteria were lysed by the French press method or by sonification. Filtered bacterial lysates were loaded on NiNTA-agarose bench top columns, washed with TBS_W (50 mM Tris, 400 mM NaCl, 20 mM imidazole, pH 7.5) and in addition washed with 70 CV PBS containing 0.1% Triton X-114 for endotoxin removal. Proteins were eluted in PBS containing 250 mM imidazole. Proteins were further purified by size exclusion chromatography using PBS buffer. Limulus amebocyte lysate (LAL)-assays were performed to assess endotoxin content. Protein concentrations were determined by absorbance spectroscopy at 280 nm and or by a BCA-assay.

Further bispecific agents are described in Examples 5 and 6.

Analysis of Bispecific Binding Reagents

Bispecific binding reagents passed quality control measurements for molecular weight, monomeric status and binding to ECD HER2. Bispecific binding agents comprising trastuzumab-competing binders (in the example, DARPin H14) also compete with trastuzumab in the bispecific setup, as expected. Bispecific binders that do not contain a trastuzumab-competing unit did not show competition in the bispecific setup, also as expected. Competitive binding ELISA, using full length HER2 ECD as target, was performed with all bispecific binding agents also versus pertuzumab. None of the bispecific binding agents competes with pertuzumab for binding to full length ECD HER2 in ELISA. Binding to the surface of viable HER2 overexpressing cancer cells was shown by flow cytometry.

For determination of the anti-tumor activity of the bispecific agents (FIG. 8), BT474 cells were seeded into 96 well plates 16 h before treatment at a density of 10×10⁴ per cm² in RPMI1640 containing 10% FCS. Titrations from 100 pM to 1 μM of each agent (final concentrations) were added and cells were treated for 4 days in a cell culture incubator. XTT viability assays were used according to the manufacturer's protocol to assess the remaining viability of the cancer cells. The targeting agents can be grouped according to their anti-tumor activity. The single binding agents scFv 4D5 and DARPin H14 reduced the cell growth by a similar extent, by 20-30%. Trastuzumab reduced the cell growth by an extent of approx. 50%. The flexible bispecific agents 926-FL-G3 and 929-FL-H14 reduced the cell growth by a similar extent of 80-90%.

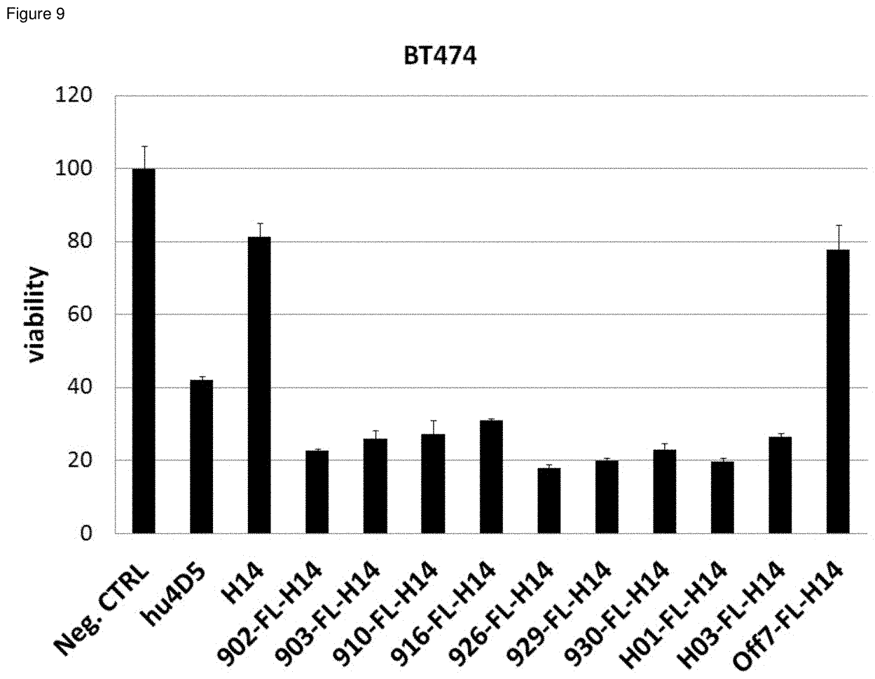

All bispecific constructs that share a similar epitope with e.g. monovalent DARPin 929 on domain 1 of HER2 ECD show strong anti-tumor activity in cell proliferation assays (FIG. 9). BT474 cells were seeded into 96 well plates 16 h before treatment at a density of 10⁴ per cm² in RPMI1640 containing 10% FCS. Anti-HER2 binding agents were added to a concentration of 100 nM (final concentration), and cells were treated for 4 days. XTT cell proliferation assays were developed according to the manufacturer's protocol. All bispecific agents containing 9XX at the N-terminus, which showed competitive binding with 926 and 929 in ELISA to ECD HER2, reduced the viability of the cancer cells by 70-80%, i.e. to a higher extent than trastuzumab.

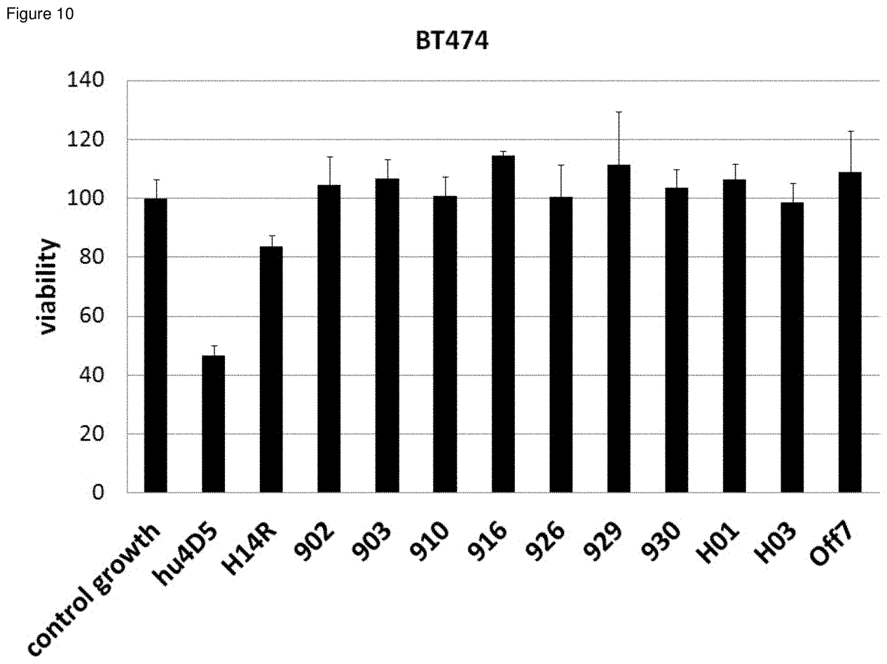

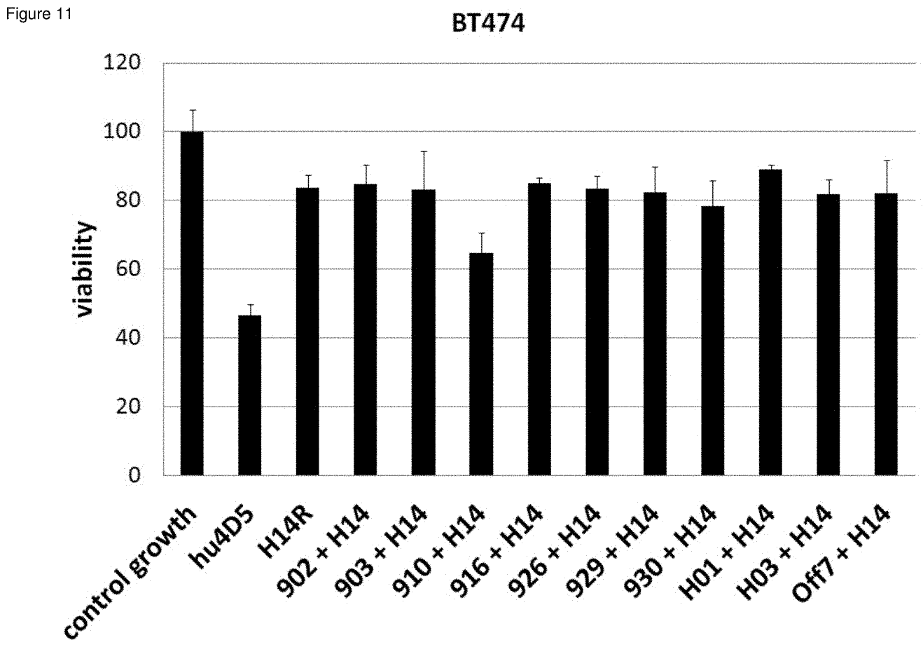

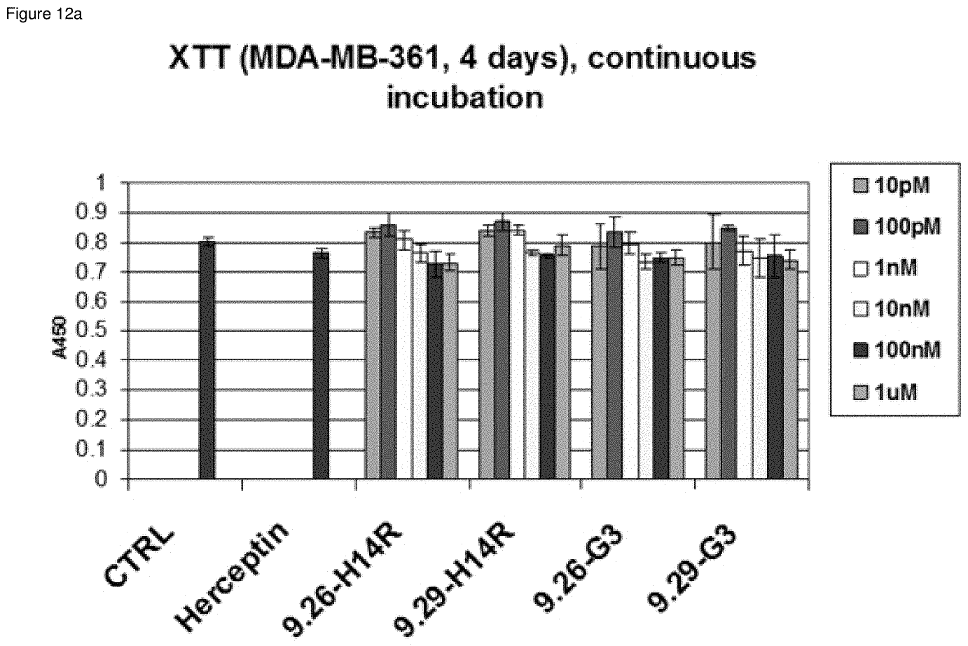

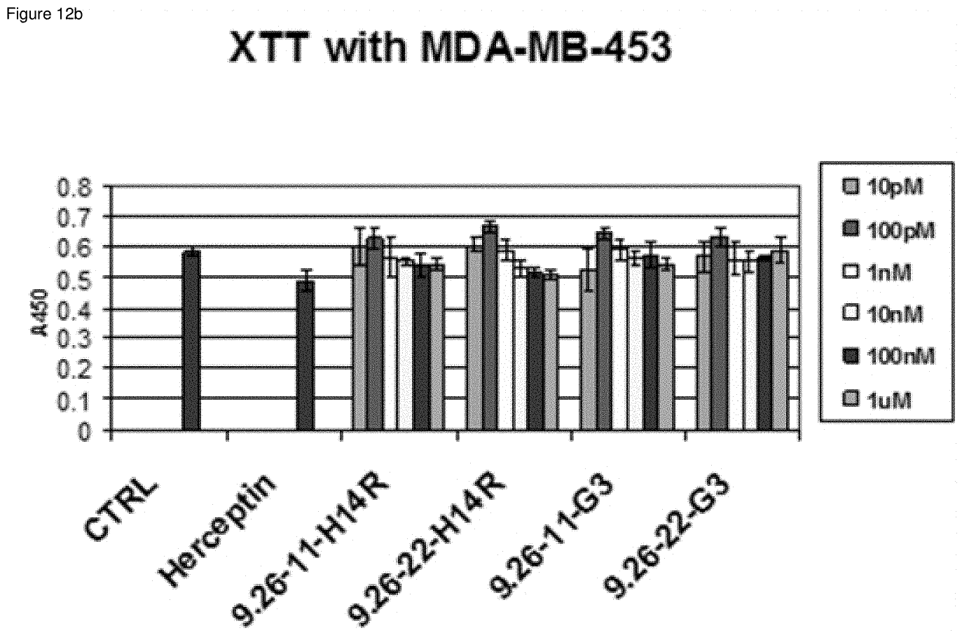

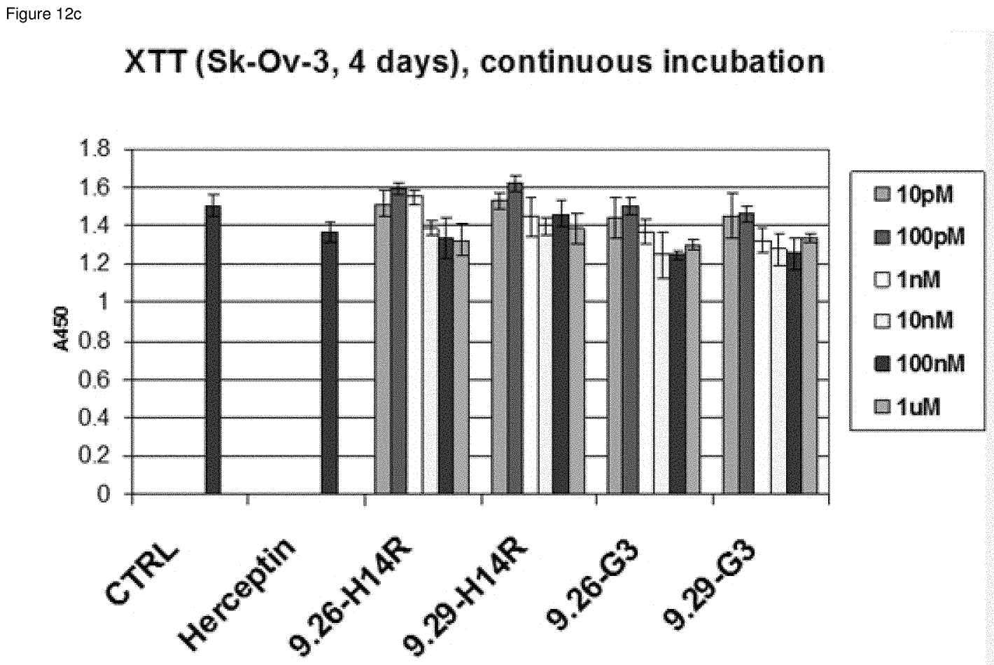

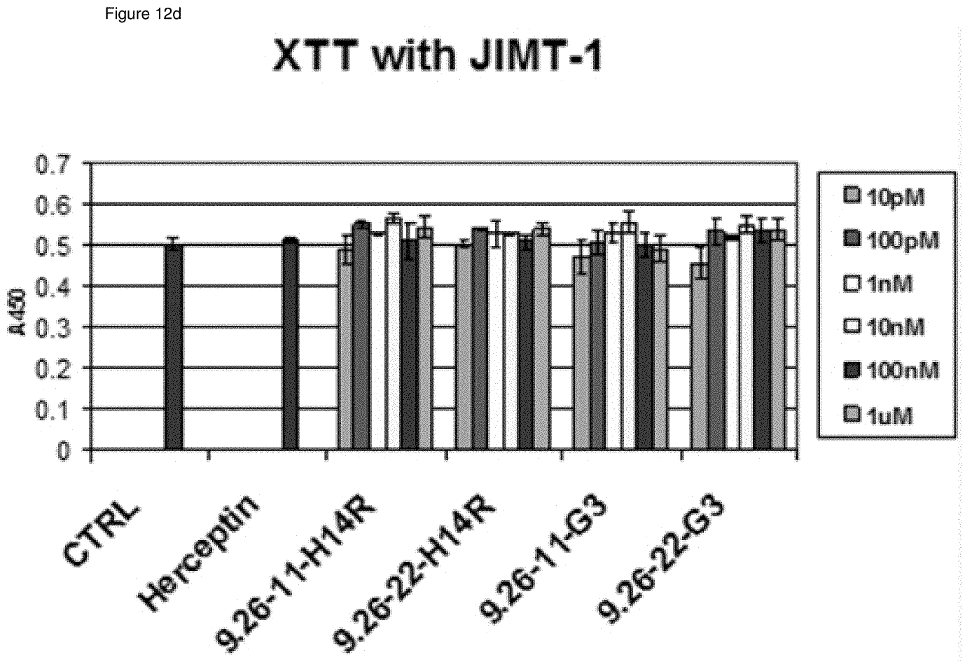

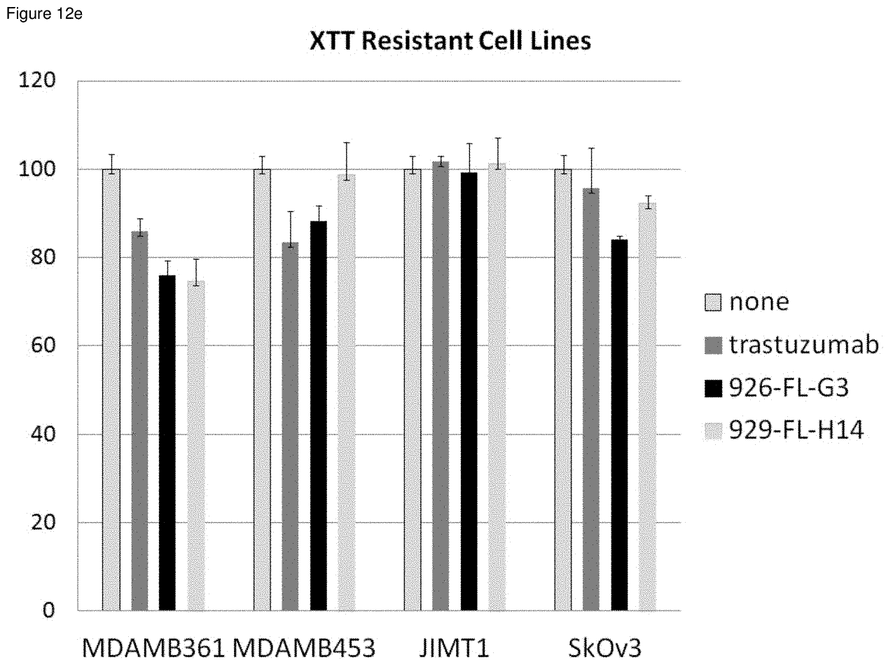

For determination of the anti-tumor activity of single binding agents, BT474 cells were seeded into 96 well plates 16 h before treatment at a density of 10⁴ per cm² in RPMI1640 containing 10% FCS. Anti-HER2 binding agents were added to a concentration of 100 nM, and cells were treated for 4 days. XTT cell proliferation assays were developed according to the manufacturer's protocol. H14, the HER2 domain 4 binding agents which competes for binding with trastuzumab (hu4D5), reduces tumor growth by 20%. The 9XX domain 1 binding agents do not show any anti-tumor activity as single binding agents (FIG. 10). The combination treatment of the single anti-HER2 binding agents is shown in FIG. 11. BT474 cells were seeded into 96 well plates 16 h before treatment at a density of 10⁴ per cm² in RPMI1640 containing 10% FCS. Anti-HER2 binding agents were added to a concentration of 100 nM, and cells were treated for 4 days. XTT cell proliferation assays were developed according to the manufacturer's protocol. The 9XX domain 4 binding agents do not show an additive effect to anti-tumor activity of H14. Thus, the strong anti-tumor activity requires that the binding agents are connected into a bispecific molecule. Cell proliferation assays with trastuzumab-resistant cell lines are shown in FIG. 12. Cancer cells were seeded into 96 well plates 16 h before treatment. A serial dilution of anti-HER2 binding agents was added and cells were treated for 4 days. XTT cell proliferation assays were developed according to the manufacturer's protocol. The anti-tumor activity of bispecific targeting agents is similarly modest to trastuzumab in trastuzumab-resistant cell lines.

### Example 3: Differentiation from Prior Art Constructs: Comparison of Apoptosis Induced by 7C2 in Combination with 4D5 Versus Bispecific Targeting Agents

As was demonstrated in the patent (U.S. Pat. No. 7,371,376 B1; US20110033460 (A1) ANTI-ErbB2 ANTIBODIES), the antibody 7C2 is competent as a single agent to induce apoptosis in the following cell lines BT474, SkBr3, SkOv3 or Calu-3. The epitopes on domain 1 of the ECD HER2 bound by 7C2 and 7F3 are different from the epitopes bound by the 9XX collection (see below), and are also different from those of scFv fragment A21 (see example 5 below) The bispecific targeting agents disclosed here induce apoptosis in BT474 and SkBr3 cells, but not in SkOv3 cells. The absence of anti-tumor activity in SkOv3 cells can be explained by the activating mutation H1047R of the PI3-Kinase. The induction of apoptosis by the bispecific targeting agents is thus correlated with a non-mutated, wild-type downstream signaling pathway of HER2 and HER3.

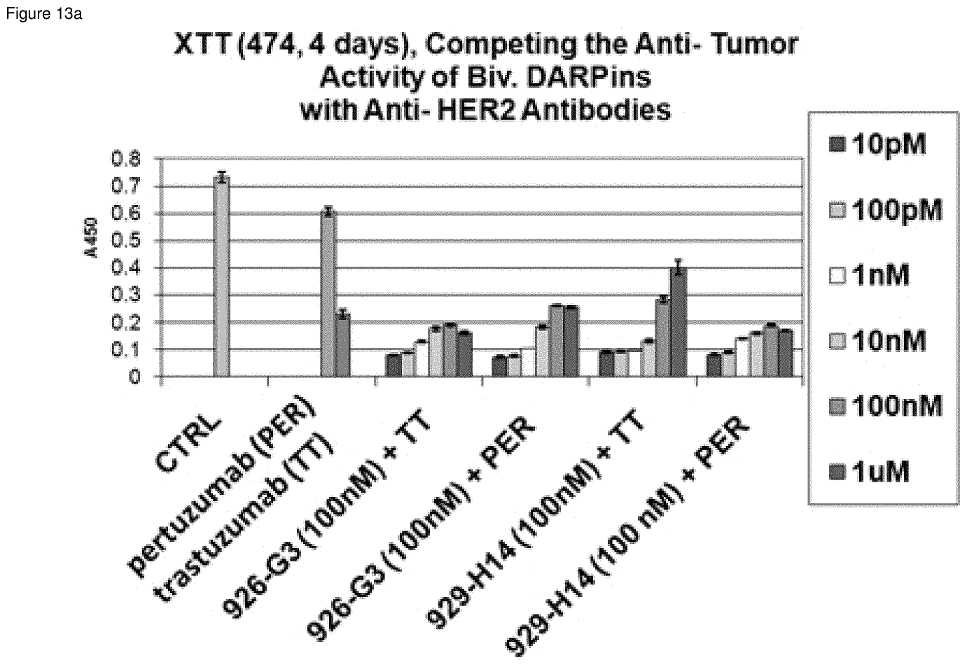

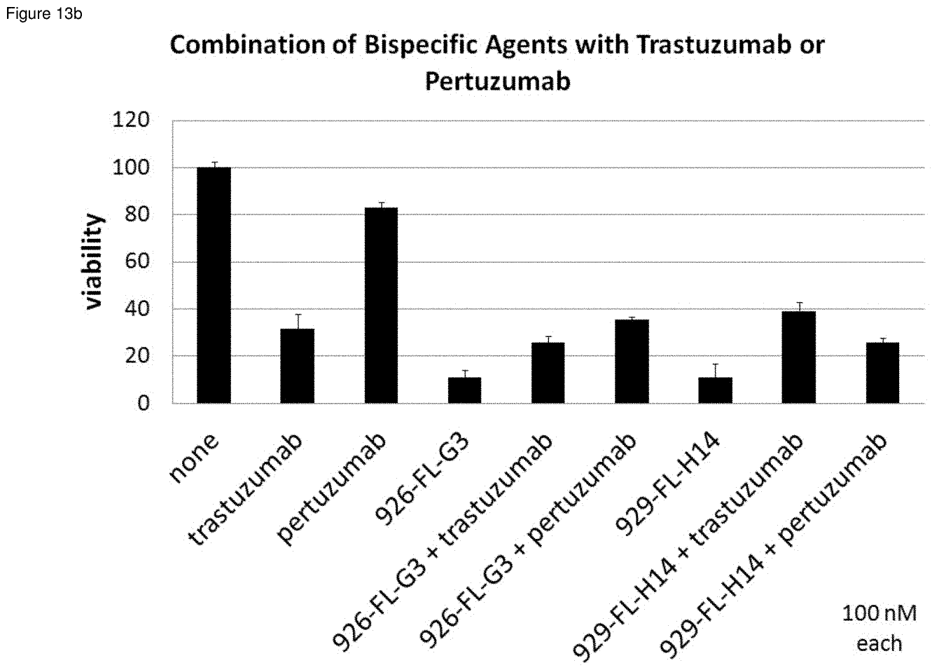

The absence of anti-tumor activity is another difference to the antibodies 7C2 and 7F3, which show anti-tumor activity as single agents. In US20110033460A1, an additive effect of 7C2 and 4D5 (trastuzumab) to anti-tumor activity is shown. In contrast, the anti-tumor activities of bispecific targeting agents disclosed here are significantly reduced in combination with trastuzumab (FIG. 13).

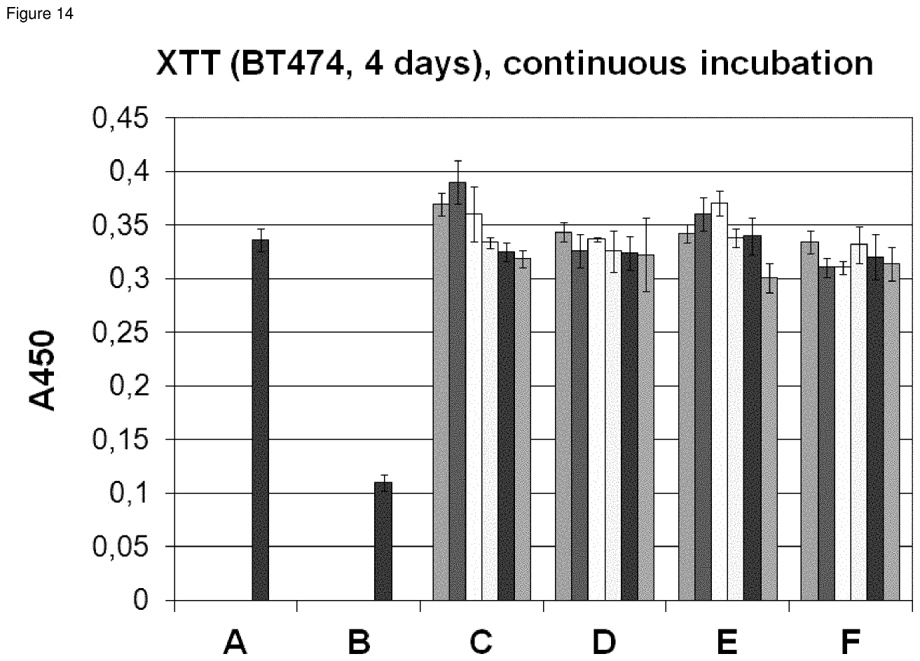

Even more importantly, the monospecific, bivalent constructs made in analogy to the targeting agents disclosed here, are not active when mixed (FIG. 14; see detailed description of this experiment below). This is in contradistinction to the mixture of the antibody 7C2 with 4D5 and 7F3 with 4D5. This underlines that the mechanism of action of said antibody mixtures is completely different to the bispecific targeting reagents disclosed herein. For the bispecific targeting reagents disclosed herein, the covalent linking of a domain I binding unit to a domain IV binding unit is essential for the mode of action.

In the case of H14 fusions this reduced activity can be explained by simple competition for binding to the same epitope, while in the case of G3 fusions, trastuzumab and G3 do not compete for binding to domain 4. Hence, trastuzumab blocks the formation of inactive HER2 homodimers that are induced by the bispecific molecules according to the invention. Therefore, the modes of action of 7C2 in combination with 4D5, in comparison to the bispecific targeting agents according to our invention, are different. Furthermore, the concept for induction of apoptosis in HER2 overexpressing cancer cells is completely different. Here it is shown that through the strong inhibition of the internal cell signalling in these HER2-dependent cancer cells, apoptosis is induced by the bispecific binding molecules. In contrast, 7C2, a homobivalent IgG, is shown to induce apoptosis but not inhibition of cell growth. This mode of action uncouples signalling from apoptosis and is therefore more similar to e.g. death receptor signaling (FAS or TNF receptor). The inventors believe, without wishing to be bound by theory, that the bispecific reagents according to the present invention work mainly by preventing formation of active dimers and act thus at the level of signaling. Downregulation of receptors is not likely to form an intrinsic part of the mechanism of the bispecific molecules disclosed here. In contrast, it may be part of the mechanism of action of the combination of 7C2 in combination with 4D5.

The antibodies trastuzumab (TT, 4D5) and pertuzumab (PER, 2C4) disrupt the inactive HER2 homodimers formed by bispecific targeting agents (FIG. 13). BT474 cells were seeded into 96 well plates more than 16 h before treatment at a density of 10⁴ per cm² in RPMI1640 containing 10% FCS. The bispecific targeting agents 926-G3 and 929-H14 were added at a concentration of 100 nM. Subsequently, titration from 10 pM to 1 μM of an anti-HER2 antibody, either trastuzumab (TT, 4D5) or pertuzumab (PER, 2C4), was added. BT474 cells were treated for 4 days in a cell culture incubator at 37° C. and 5% CO₂. XTT cell viability assays were performed according to manufacturers protocol. The absorbance at 450 nm correlates with the number of viable cells. By increasing concentrations of trastuzumab or pertuzumab in the presence of the bispecific agents 926-G3 and 929-H14, the antitumor activity of the bispecific targeting agents is significantly reduced. This indicates that the anti-tumor effect of the bispecific molecules according to this invention is greater than that of trastuzumab or pertuzumab.

The anti-tumor activity of bispecific targeting agents is not caused by random cross-linking of receptors (FIG. 14; A-control; C—926-22-926/H14R-22-H14R; D—926AvantE-22-926AventE/H14R-22-H14R; E—926AvantE-22-926AventE/H14AvantE-22-H14AventE; F—926-22-926/G3-22-G3; first column=10 pM, second column=100 pM, third column=1 nM, forth column=10 nM, fifth column=100 nM and sixth column=1 μM of C, D, E and F, respectively). BT474 cells were seeded into 96 well plates more than 16 h before treatment at a density of 10⁴ per cm² in RPMI1640 containing 10% FCS. Combinations of homo-bivalent targeting agents were titrated from 10 pM to 1 μM. The combination of both homo-bivalent targeting agents did not show any signification reduction in the viability of the cancer cells.

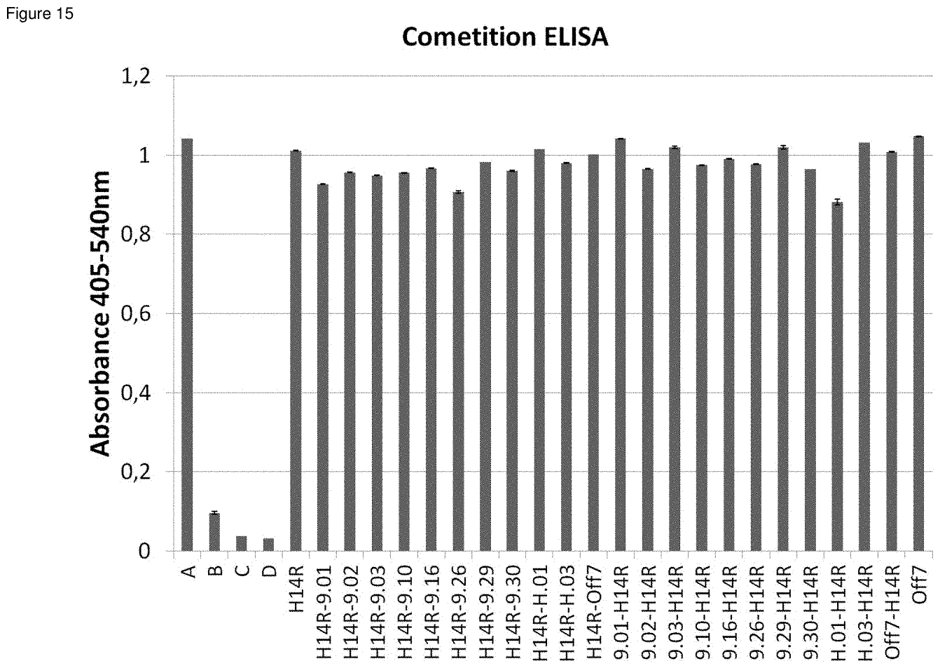

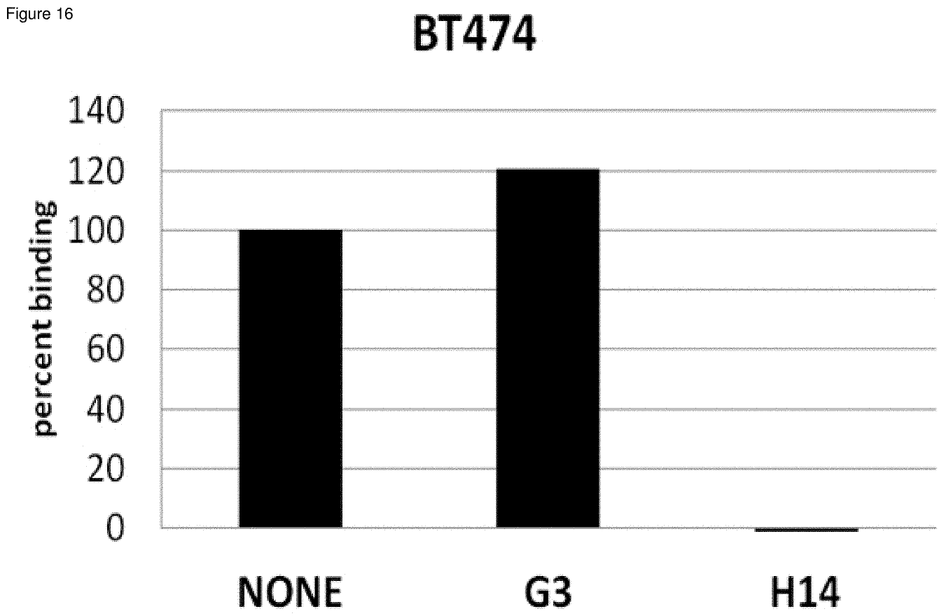

Bispecific targeting agents do not compete for binding with pertuzumab in ELISA (FIG. 15, A-pertuzumab, 2ndAb (no competitor), B-2nd Ab, C-pertuzumab, 2nd Ab (no ErbB2), D-2nd Ab (no ErbB2)). Wells of the MaxiSorp plate were coated with 100 μl PBS containing 66 nM streptavidin for 12 hours at 4° C. Liquids were removed completely after each step. The plastic surface was blocked by PBS_TB (PBS containing 0.1% Tween20, 0.2% BSA) for 1 hour at room temperature with continuous shaking. Afterwards, 20 nM of truncated ErbB2-avidin conjugate was added in 100 μl PBS_TB and incubated for 1 hour. The plate was washed four times with PBS_TB. Then, bivalent DARPins were added to 1 μM in PBS_TB, and binding took place for 3 hours on a shaker. Next, 1 nM of pertuzumab was added and incubated for 30 min. The plate was washed four times in PBS_TB. The secondary anti-human antibody coupled to alkaline phosphatase was incubated in 100 μl PBS_TB for 1 hour. The plate was washed four times with PBS_TB. Finally, 100 μl of freshly prepared and filtered pNPP buffer (3 mM pNPP, 50 mM NaHCO₃, 50 mM MgCl₂) was added and the color reaction was developed for 5 min at room temperature. Absorbance was detected on an ELISA plate reader at the wavelength of 405 nm. Analysis of competitive binding to domain 4 of HER2 was measured by flow cytometry (FIG. 16). 10⁵ BT474 cells were incubated with either 1 μM of G3 or H14 for 30 min at room in 100 μl PBS_BA (PBS, 0.2% NaN₃, 1% BSA). Subsequently, Alexa₄₈₈-trastuzumab, which had been labeled with Alexa₄₈₈-succinimidyl ester, was added to a concentration of 100 nM and incubated for 30 min at room temperature. Afterwards, cells were washed twice using PBS_BA. Flow cytometry measurements were performed on a Cyflow space system. 10⁴ events were recorded in a FSC/SSC gate to measure cells with proper size. Mean fluorescence intensities were calculated by FlowJo software and data were normalized to the MFI of Alexa₄₈₈-trastuzumab binding. G3 does not compete with the binding of trastuzumab, while H14 and trastuzumab bind to a very similar epitope and therefore show 100% competition for binding.

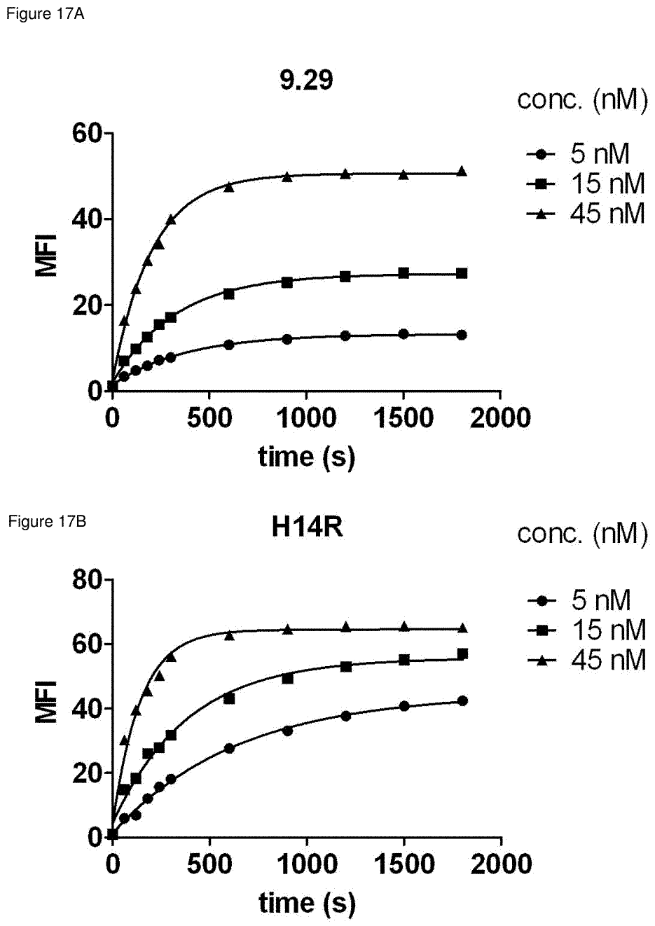

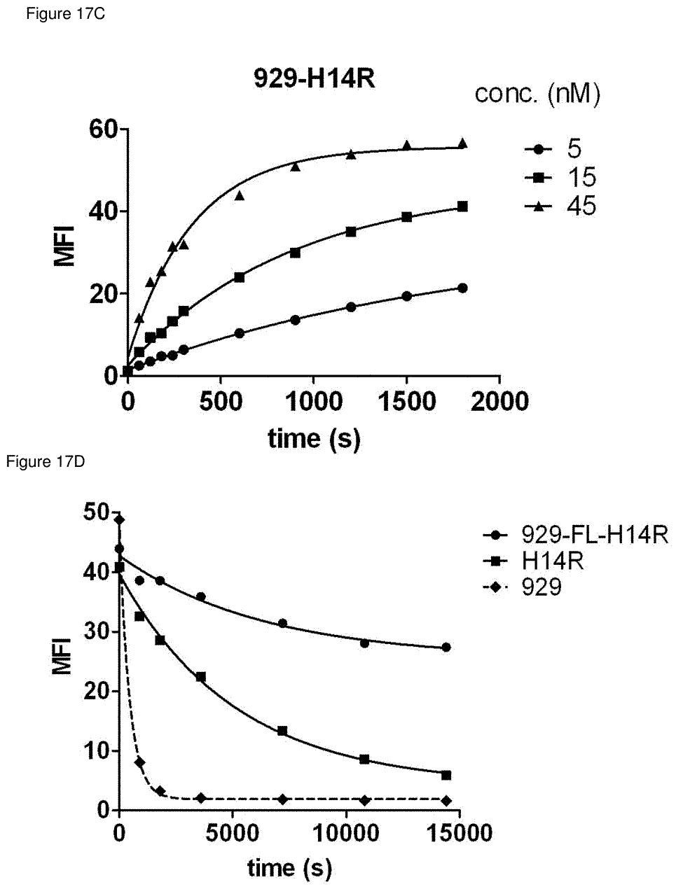

Bivalent binding of the bispecific targeting agent to HER2 at the surface of cancer cells is a prerequisite for strong anti-tumor activity. To confirm the binding of bispecific agents, the association rate constant kₒₙ and dissociation rate constant k_{off} on intact cells can be measured by flow cytometry (FIG. 17) (Tamaskovic et al. (2012) Methods Enzymol. 503, 101-134).

The following tables show the determined binding affinities of single and bispecific binding agents and certain DARPins.

| | average | average | average | average |
|---|---|---|---|---|
| | kₒₙ (M⁻¹s⁻¹) | k_{obs} (s⁻¹) | k_{off} (s⁻¹) | K_{d} (M) |
| 929 | 68977 | 0.0035 | 2.21 × 10⁻³ | 33.47 × 10⁻⁹ |
| H14 | 196244 | 0.0037 | 1.79 × 10⁻⁴ | 0.97 × 10⁻⁹ |
| 929-FL-H14 | 77959 | 0.0015 | 3.99 × 10⁻⁵ | 0.52 × 10⁻⁹ |

| DARPin | K_{D} (nM) | kₒₙ (10⁵M⁻¹s⁻¹) | k_{off} (10⁻³s⁻¹) |
|---|---|---|---|
| 916 (domain 1 binder) | 6.9 | 1.2 | 0.9 |
| 926(domain 1 binder) | 1.4 | 0.7 | 0.1 |
| 929 (domain 1 binder) | 3.8 | 2.0 | 0.8 |
| H14 (domain 4 binder) | 0.2 | 4.1 | 0.1 |

Preparation of Cancer Cells for Flow Cytometry Measurements

Cells were detached by collagenase and EDTA for 5 min at 37° C. The solution was quenched by addition of medium and centrifuged at 300 g for 3 min. Cells were washed twice in warm PBS. Cell densities were determined with a CASY cell analyzer and adjusted to 10⁶ cells per sample. Internalization was blocked by incubation in PBS containing 0.2% NaN₃ and 1% BSA for 30 min at 37° C.

Flow Cytometry Measurements

Samples were resuspended in 1 ml cold PBS and measured on flow cytometer. 10,000 cells per sample were recorded. Results were gated for FSC vs SSC of the cells. Green fluorescence was detected with the FL1 detector. Data were processed by the FlowJo 7.2.5 software.

Measuring Association of Binding Agents on the Surface of Cancer Cells

For on-rate determinations, BT474 cells are incubated at a concentration of 1×10⁶ cells/ml with 2.5, 7.5, and 22.5 nM DARPin-Alexa Fluor-488 conjugates in PBSBA at room temperature for defined time intervals, ranging from 1 to 60 min. For each time point, a 1 ml aliquot of cells is withdrawn and subjected to FACS. Since the applied concentrations of the labeled ligand conjugates are very low, and since the time resolution of the measurement is to be maintained to ensure the accuracy of the on-rate determination, the samples are processed without further washing. For each time point, at least 10⁴ intact cells (gated as a uniform population on a FSC/SSC scatter plot) are counted, and the MFI (mean fluorescence intensity) is recorded.

Measuring Dissociation of Binding Agents on the Surface of Cancer Cells

10⁶ cells per time point were incubated with 1 μM Alexa488 labeled binding agents in 100 μl PBS (0.2% NaN₃, 1% BSA) for 1 hour at 4° C. on the shaker. Corresponding to 100 μl cell suspension, samples were washed twice in 1 ml PBS (0.2% NaN₃, 1% BSA) and centrifuged at 600 g for 30 sec at room temperature. Cells were resuspended in 1 ml PBS (0.2% NaN₃, 1% BSA) containing 100 nM of equivalent unlabeled binding agent. The dissociation reaction was incubated for the indicated times (15, 30, 60, 120, 180 and 240 min) at room temperature while continuously stirring in the dark. Dissociation was stopped by placing the cell pellets on ice. Each sample was washed once with 1 ml cold PBS.

### Example 4: Additional Data Regarding Construction and the Effects of Mono- and Bivalent Constructs on Cell Proliferation and Cell Death

DARPins that had been selected by phage display or ribosome display to target the full-length ectodomain of HER2 without showing any cross-specificity against other EGFR-family members were characterized concerning which of the four HER2-subdomains forms the epitope. Since DARPins typically recognize conformational epitopes, subdomains were expressed alone and in combination in insect cells using a baculovirus system. To minimize glycosylation for subsequent crystallization, the Asn residues were replaced in predicted N-linked glycosylation sites by Asp. ELISAs on these proteins showed that the epitopes recognized by DARPins 9_26 and 9_29 are located on HER2-I, while DARPin G3 bound to HER2-IV. Competition for binding to HER2-overexpressing cells measured by flow cytometry revealed that DARPins 9_26 and 9_29 compete for the same epitope. DARPin G3, which binds to HER2 subdomain IV, did not compete with trastuzumab but competed with a different HER2-specific DARPin, H.14, which in turn competed with trastuzumab.

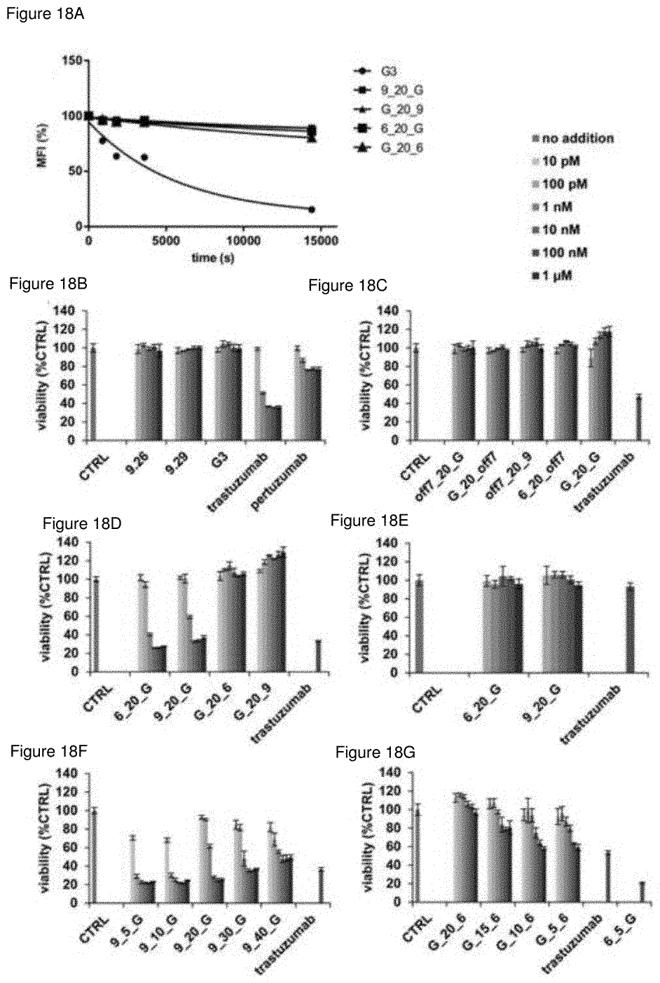

Various bivalent and bispecific constructs were generated by genetically fusing two DARPins by (G₄S)ₙ linkers of different lengths. To target two non-overlapping epitopes with a single molecule, DARPins 9_29 or 9_26 were connected to DARPin G3 by a 20 amino acid linker, with either an ECD-I binder at the N-terminal end and the ECD-IV binder at the C-terminus or in opposite orientation. The four different bispecific binders (e.g., 9_26-(G₄S)₄-G3, abbreviated “6_20_G” for the two DARPins and the linker length of 20 amino acids) were tested regarding their binding to HER2-overexpressing cells. G3 with a KD of 90 pM has the highest affinity of the three HER2-binders used in this study, compared to a KD of 1 nM for 9_26 and 1 nM for 9_29. Kinetic experiments on cells in the presence of a competing DARPin (to prevent rebinding) revealed that the off-rates of the bispecific binders were 10 times lower than the off-rates of monovalent G3 (FIG. 18A). The slower off-rate and higher KD of the bispecific constructs, compared to their monovalent building blocks, can be attributed to an avidity effect and indicates bispecific binding to HER2 on the cell.

The influence of the different DARPin constructs on cell proliferation and cell survival were tested in XTT assays, using BT474 cells as an example of a HER2-addicted cell line. MCF7-cells, which express HER2 at much lower levels than BT474 cells, were used as a control. Calibration experiments showed that a signal decrease by 60%, compared to untreated cells, corresponded to lack of cell proliferation over the 4 days of cell growth before the XTT assay—a larger decrease indicated cell death. The XTT assay were performed as described in example 1.

None of the characterized monovalent DARPins affected the number of viable cells measured by the XTT assay (FIG. 18B). Mixtures of two different DARPins proved to be equally inert, as did control constructs in which one of the two DARPins in the bispecific molecule had been replaced by a non-HER2-binding DARPin (DARPin off7, targeting maltose-binding-protein) (FIG. 18C). A monospecific bivalent DARPin G_20_G even stimulated cell proliferation (FIG. 18C).

Bispecific constructs composed of a subdomain I binder at the N- and the subdomain IV binder at the C-terminus (6_20_G or 9_20_G) showed a concentration-dependent decrease of cell viability by up to 75%, while treatment with trastuzumab decreased viability by ˜50% (FIG. 18D). The constructs with reverse orientation (G_20_9) either lacked any effect on cell-growth (G_20_6) or even slightly promoted cell growth. Similar to trastuzumab, bispecific constructs did not affect the cell-proliferation of MCF7-cells (FIG. 18E), suggesting the restriction of the observed effects to HER2-addicted cells. Comparison of constructs with 5, 10, 20, 30 and 40 amino acid linkers showed that for 9_x_G constructs, specific activity and potency decreases with increasing linker length. The most potent constructs proved to be 6_5_G and 9_5_G, with (G₄S)-linkers of only five amino acids. They decreased the cell viability in XTT-assays after four days of growth by more than 80% as compared to untreated cells, and showed a half-maximal effect already at a concentration of less than 100 pM compared to ca. 1 nM for 6_20_G and 9_20_G. Conversely, increasing the linker length to forty amino acids, as in 6_40_G and 9_40_G, decreased the biological activity (growth reduction of only 40%) (FIG. 18F). The constructs with inverse orientation, G_x_6 and G_x_9, inactive or even stimulatory at a linker length of 20 amino acids, gained anti-proliferative activity at short linker lengths, but the best construct was found to be only as active as trastuzumab (FIG. 18G).

Neither the single DARPins nor the bispecific constructs affected internalization or degradation of HER2, as determined by flow cytometry.

### Example 5: Bispecific HER2 Bindings Agent with One or Two Antibody Fragments

To demonstrate the cytotoxic activity of bispecific HER2 binding agents constructed from antibody fragments, bispecific constructs of the type scFv1-linker-scFv2; DARPin-linker-scFv; and scFv-linker-DARPin constructs were constructed. Here, in each fusion protein, one of the units (scFv1, scFv2, scFv or DARPin) binds to domain 1, the other one binds to domain 4.

For a description of scFv1-linker-scFv2 constructs, cf. p. 37.

To generate a domain 1-binding scFv, the scFv chA21 (A21) was used described in Hu S. et al., (2008) Proteins 70:938-949.). The crystal structure in complex with HER2 had been determined, verifying the binding of this scFv to domain 1. The protein sequence of the heavy and light chain of the scFv A21 was obtained from the PDB file (PDB ID: 2GJJ). A flexible glycine serine linker of 4×GGGGS units (GGGGS GGGGS GGGGS GGGGS, SEQ ID 54) was introduced to connect the heavy and the light chain in either orientation: Two orientations were thus obtained, by either fusing the N-terminal heavy chain to the light chain (A21 HL, SEQ ID 65) or the N-terminal light chain to the heavy chain (A21 LH, SEQ ID 66 or SEQ ID 93) within one single protein sequence connected by the said glycine-serine linker.

The respective gene sequences were synthesized by Genescript Inc., and they additionally contain a BamHI/HindIII cloning site for directional cloning (see below).

To generate a domain 4-binding scFv, the scFv of the antibody hu4D5 was constructed. The crystal structure of the corresponding Fab fragment (hu4D5, trastuzumab; Herceptin) in complex with HER2 had been determined, verifying the binding of this scFv to domain 4, as described in Cho et al., (2003) Nature 421:756-760. The protein sequence of the heavy and light chain for the construction of the scFv 4D5 was obtained from the PDB file (PDB ID: 1N8Z). A flexible glycine serine linker of 4×GGGGS units (GGGGS GGGGS GGGGS GGGGS) was introduced to connect the heavy and the light chain in either orientation: Two orientations were thus obtained, by either fusing the N-terminal heavy chain to the light chain (4D5HL, SEQ ID 67) or the N-terminal light chain to the heavy chain (4D5LH, SEQ ID 68 or SEQ ID 92) within one single protein sequence connected by the said glycine-serine linker. Also, an additional scFv 4D5LH (SEQ ID 69) with an alternative has been created.

The respective gene sequences were synthesized by Genescript Inc., and they additionally contain a BamHI/HindIII cloning site for directional cloning (see below).

Construction of scFv1-Linker-scFv2; DARPin-Linker-scFv; and scFv-Linker-DARPin Fusion Proteins

For the gene construction of bispecific fusions proteins, which contain a HER2 domain 1 and a domain 4 binding moiety, a generic vector (pMxAC) was used. This vector is based on pMx9 (Rauchenberger, R. et al. (2003) J. Biol. Chem. 278, 38194-38205), and contains an OmpA signal sequence for periplasmic expression in E. coli. The OmpA signal sequence was exchanged by a DsbA signal sequence taken from the vector pDSt066 (see description in Steiner et al. (2008) J. Mol. Biol., 382:1211-1127). In addition, a new multiple cloning site was introduced into the vector pMx9 containing the DsbA signal sequence, in which restriction sites allowed specific cloning on either side of the flexible gly-ser linker. These cloning cassettes therefore allowed the preparation of fusion proteins with different lengths of linkers originating from the plasmid pQiBi-22-(4×GGGGs flexible linker, SEQ ID 54); pQiBi-11-(2×GGGGs flexible linker, SEQ ID 52) and pQiBi-01-(1×GGGGs flexible linker, SEQ ID 51) (Boersma et al. (2011), J. Biol. Chem. 286, 41273-41285.)

The new vectors were termed pMxAC-22-(4×GGGGs flexible linker, SEQ ID 54); pMxAC-11-(2×GGGGs flexible linker, SEQ ID 52) or pMxAC-01-(1×GGGGs flexible linker SEQ ID 51) respectively.

These pMxAC vectors contain a BamHI/HindIII cloning site for inserting the N-terminal binding construct (upstream of the linker) and a BgIII/BsaI site (compatible with BamHI/HindIII cloning sites) cloning site for introducing the C-terminal binding moiety (downstream of the linker). In addition, the construct contains a C-terminal 6×His-tag for purification and detection and a FLAG-tag M1 for detection of periplasmic export (Knappik et al. (1994) Biotechniques 17, 754-761.).

Map of the ORF in the pMxAC-22- vector
(SEQ ID 94)
MKKIWLALAGLVLAFSASADYKDDIGS

[symbol]

(SEQ ID 95)
KLGGGGSGGGGSGGGGSGGGGSRS

[symbol]

(SEQ ID 96)
KLGSHHHHHH
Legend, explaining the different elements:
MKKIWLALAGLVLAFSASA: DsbA-signal sequence, which
gets cleaved off
DYKDDI: FLAG-Tag M1
GS: BamHI cloning site [symbol]
N-terminal protein of interest,
either scFv or DARPin
KL: HindIII cloning site
GGGGSGGGGSGGGGSGGGGS: Flexible linker (-22-/FL4,
SEQ ID 54)
RS: BgIII cloning site [symbol]
C-terminal protein of interest,
either scFv or DARPin
KL: Bsa1 cloning site
GS: flexible spacer
HHHHHH: 6xHis-Tag

Alternative Vectors for scFv/DARPin Fusion Proteins

In addition to the periplasmic expression in E. coli described above, expression of the scFv/DARPin fusion proteins was performed by secretion from Spodoptera frugiperda (Sf9) cells using the Multibac system as described previously (Fitzgerald et al. (2006) Nature Methods 3:1021-32.). In brief, the coding sequences of the fusion proteins were subcloned via ligation-independent cloning (LIC) into the donor vector pFLmLIC introducing an N-terminal melittin signal sequence (SEQ ID 99). The donor vectors were used to introduce the fusion protein coding sequences into the bacmid EmBacY. Baculoviruses for infection of Sf9 cells were generated through transfection of the bacmid DNA into Sf9 cells. For expression, Sf9 cells were grown to a density of 4×10⁶ cells/mL and co-infected with the respective virus at a MOI of 1.72 h post infection, cells were harvested by centrifugation (30 min, 5000 g, 4° C.) and the cleared medium was subjected to immobilized metal ion affinity chromatography (IMAC) purification with Ni-NTA Superflow (Qiagen) purification resin.

The following table shows the scFv/DARPin fusion proteins which were expressed in Sf9 cells or in E. coli. Note that the N-terminal melittin signal sequence (MVVYISYIY, SEQ ID 99) is cleaved upon protein secretion and not present in the secreted and purified proteins.

| scFv/DARPin fusion protein | SEQ ID |
|---|---|
| A21HL_L4_G3 | 70 |
| A21LH_L4_G3 | 71 |
| A21HL_L4_H14 | 72 |
| H14_L4_A21LH | 73 |
| H14_L4_A21HL | 74 |
| G3_L4_A21LH | 75 |
| G3_L4_A21HL | 76 |
| A21HL_L1_G3 | 77 |
| 9.29_L1_4D5LH | 78 |
| 926E-L4-4D5HL | 88 |
| 926E-L4-4D5LH | 89 |
| 929-L4-4D5HL | 90 |
| 929-L4-4D5LH | 91 |

Expression scFv1-Linker-scFv2 Constructs in the Periplasm of E. coli

ScFv1-linker-scFv2 constructs were co-expressed with periplasmic chaperones in the periplasm of E. coli. For this purpose, the pMxAC scFv1-linker-scFv2 plasmids were co-transformed with the plasmid pCH-A1 (Schaefer and Plückthun (2010) Improving expression of scFv fragments by co-expression of periplasmic chaperones, in: Antibody Engineering, Kontermann, and Dübel, eds., Vol. 2, 2nd edit., pp. 345-361, Springer Verlag, Berlin Heidelberg, Germany) into E. coli SF130 (Meerman and Georgiou (1994); Biotechnology (N Y) 12:1107-1110). After transformation, single clones of E. coli were adapted to Terrific Broth growth medium (TB; Cold Spring Harbor Protocols) overnight and transferred to 1 L TB expression culture to an initial OD₆₀₀ of 0.1. ScFv fusion construct expression was induced by isopropyl-β-D-thiogalactopyranoside (IPTG), and expression was performed overnight at 25° C.

Purification of scFv1-Linker-scFv2 Constructs from E. coli Expression Culture

Expression cultures were pelleted by centrifugation, washed with Tris buffer (50 mM Tris base, 150 mM NaCl, pH 7.5) and resuspended in cold Tris buffer containing protease inhibitors (Roche—complete protease inhibitor cocktail) and DNasel (Roche) and kept at 4° C. during the entire process. E. coli were lysed with a French press and centrifuged for 30 min at 20,000 g. The supernatant was adjusted to a final concentration of 20 mM imidazole, 400 mM NaCl, 10% glycerol, pH 7.5, and applied to Ni-NTA bench-top columns. Columns were washed with 30 CV of Tris buffer containing 20 mM imidazole, 400 mM NaCl and 10% glycerol, high-salt washed with 30 CV Tris buffer containing 1 M NaCl, low-salt washed with 30 CV Tris buffer containing 10 mM NaCl. The bound fraction was eluted with Tris buffer containing 300 mM imidazole. Ni-NTA-eluted protein was loaded on a protein-A bench-top column, and endotoxin-washed with 80 CV phosphate buffer saline (Dulbecco's PBS) containing 0.1% Triton X-114, washed with 30 CV PBS and eluted with 4 CV 100 mM glycine buffer pH 3.6 into 4 CV of 1.5 M Tris buffer pH 8, 150 mM NaCl. Proteins were concentrated and dialyzed against HEPES buffer (25 mM HEPES, 150 mM NaCl, pH 7.5).

The following table shows the scFv1-linker-scFv2 constructs that have been expressed in E. coli:

| scFv1-linker-scFv2 | SEQ ID |
|---|---|
| 4D5HL-L1-A21HL | 80 |
| 4D5HL-L4-A21LH | 81 |
| 4D5LH-L1-A21HL | 82 |
| 4D5LH-L4-A21HL | 83 |
| 4D5LH-L4-A21LH | 84 |
| A21HL-L4-4D5LH | 85 |
| A21LH-L1-4D5LH | 86 |
| A21LH-L4-4D5LH | 87 |
| 4D5LH-L1-A21LH | 100 |

Diabody A21H 4D5LH A21L

The gene of the diabody construct (analogous to constructs described by Völkel et al. (2001), Protein Engineering 14, 815-823), consisting domains from scFv fragments of 4D5 and A21, was synthesized at Genescript Inc. and carries additionally BamHI/HindIII cloning sites for directional cloning into pcDNA3 (see below).

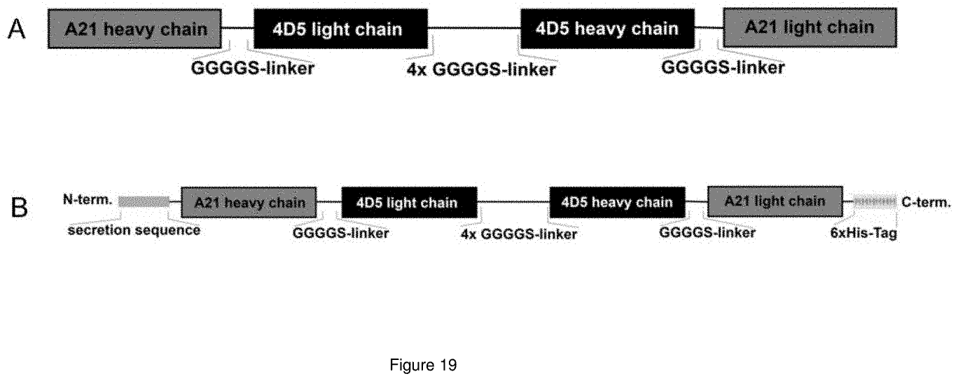

The diabody construct A21H_4D5LH_A21L (SEQ ID 79) consists of a first moiety consisting of the A21 heavy chain connected to the 4D5 light chain by a glycine/serine linker characterized by SEQ ID 51, and second moiety consisting of the 4D5 heavy chain connected to the A21 light chain by a glycine/serine linker characterized by SEQ ID 51, wherein the first moiety is connected to the second moiety by a glycine/serine linker characterized by SEQ ID 54 (FIG. 19A).

Expression of Diabody Constructs in CHO Cells

For the expression of the diabody construct A21H_4D5LH_A21L a vector plasmid based on pcDNA3.1(+) Hygro has been constructed. A poly linker (multiple cloning site) was synthesized that carries a N-terminal signal sequence of the mouse Ig Kappa light chain followed by BamHI/HindIII cloning site and a C-terminal 6×His-tag (FIG. 19B). The vector was termed pcDNA3.1 Seq mIgk.

(SEQ ID 97)
METDTLLLWVLLLWVPGSTGS

[symbol]

(SEQ ID 98)
KLHHHHHH
METDTLLLWVLLLWVPGST: mouse Ig Kappa light chain
signal sequence
GS: BamHI site
KL: HindIII site
HHHHHH: 6xHis Tag

Chinese hamster ovarian cells (CHO) FreeStyle from Invitrogen adapted for serum free suspension growth have been used for transient expression of the diabody construct. The diabody plasmid (pcDNA3.1 Seq mIgk A21H_4D5LH_A21L) was transfected into CHO cells by TransIT-PRO (Mirus) transfection reagent using the manufacturer's protocol. Expression was performed in bioreactors (Sigma) for 1 week in CHO-FreeStyle medium (Invitrogen).

Purification of Diabody Constructs from Supernatant of CHO Cells

After expression, the supernatant was collected by centrifugation, filtered and concentrated to a small volume. The supernatant was dialyzed against Tris buffer (50 mM Tris base, 150 mM NaCl, pH 7.5) and afterwards adjusted to 20 mM imidazole, 400 mM NaCl, 10% glycerol and loaded on a Ni-NTA bench top column. The column was washed with 30 CV of Tris buffer containing 20 mM imidazole, 400 mM NaCl, 10% glycerol, pH 7.5, 30 CV of Tris buffer and eluted in 2 CV Tris buffer pH 7.5 containing 300 mM imidazole. Samples were concentrated and dialyzed against HEPES buffer (25 mM HEPES, 150 mM NaCl, pH 7.5).

Anti-Tumor Activity of the Bispecific HER2 Binding Agents in Comparison to Trastuzumab

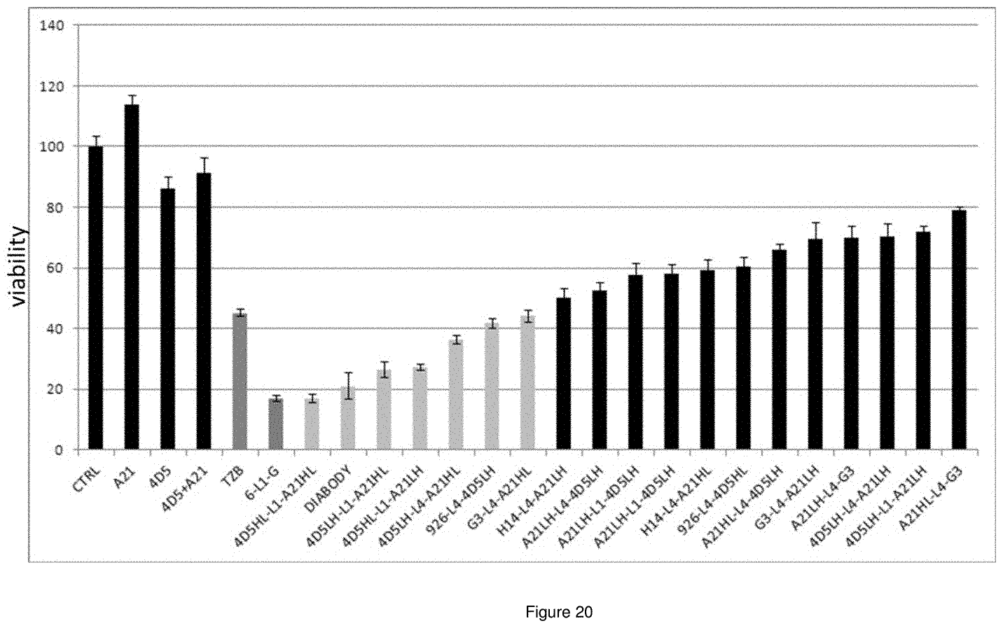

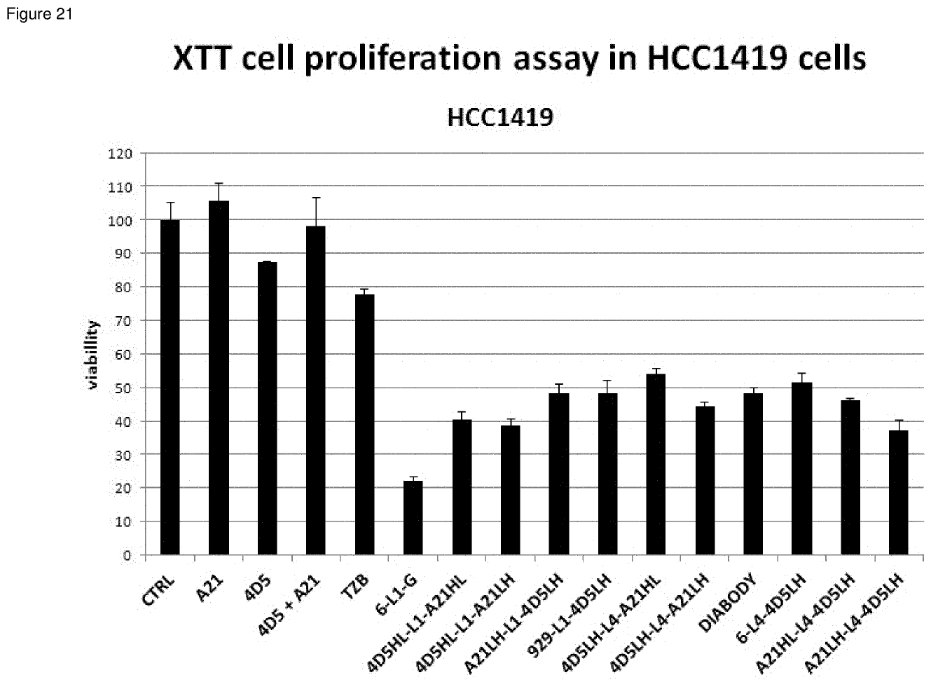

To test the cytotoxic activity of the bispecific HER2 binding agents described above, XTT-viability assay were performed as described in example 1. FIG. 20 show the results of the tests in BT474 cells, and FIG. 21 the results of the tests in HCC1419 cells, wherein CTRL means control, no addition; A21 the scFv fragment A21, 4D5 the scFv fragment 4D5; A21+4D5, a mixture of scFv fragment A21 and scFv fragment 4D5; and TZB trastuzumab. Note that the diabody (SEQ ID 79) was used at only 10 nM in the experiments shown in FIG. 21, while all other agents were used at 100 nM.

These results show that the principle of connecting a binder of domain 1 of HER to a binder of domain of HER2 by a linker leads in order to obtain a compound with strong cytotoxic and/or anti-proliferative effects does work, no matter whether the binder is or comprises an antibody fragment or a DARPin.

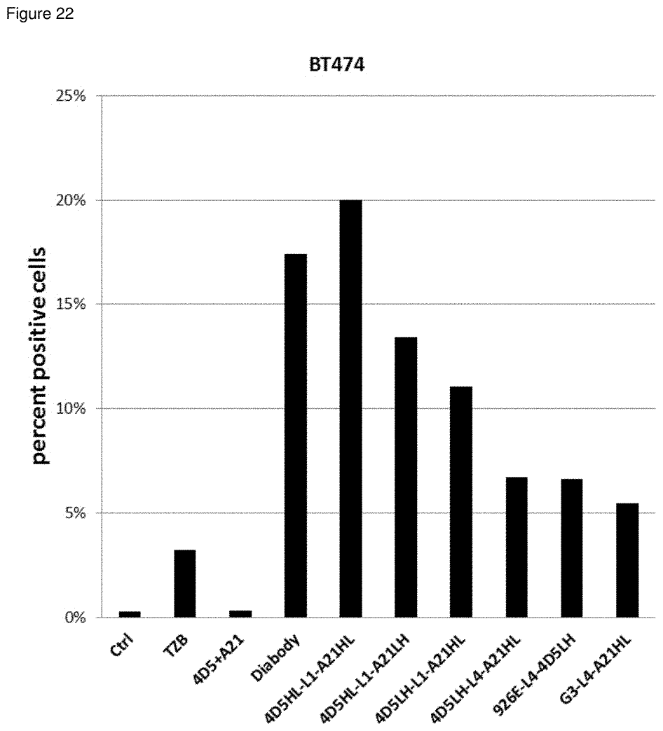

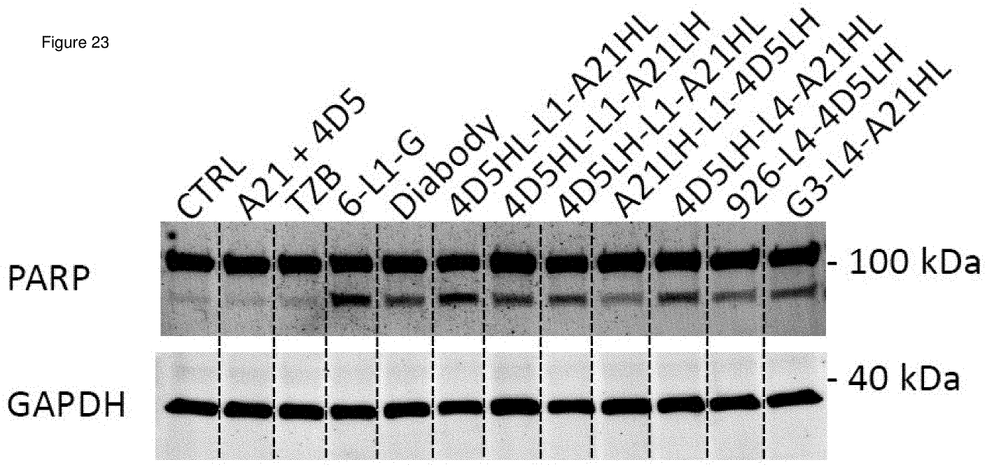

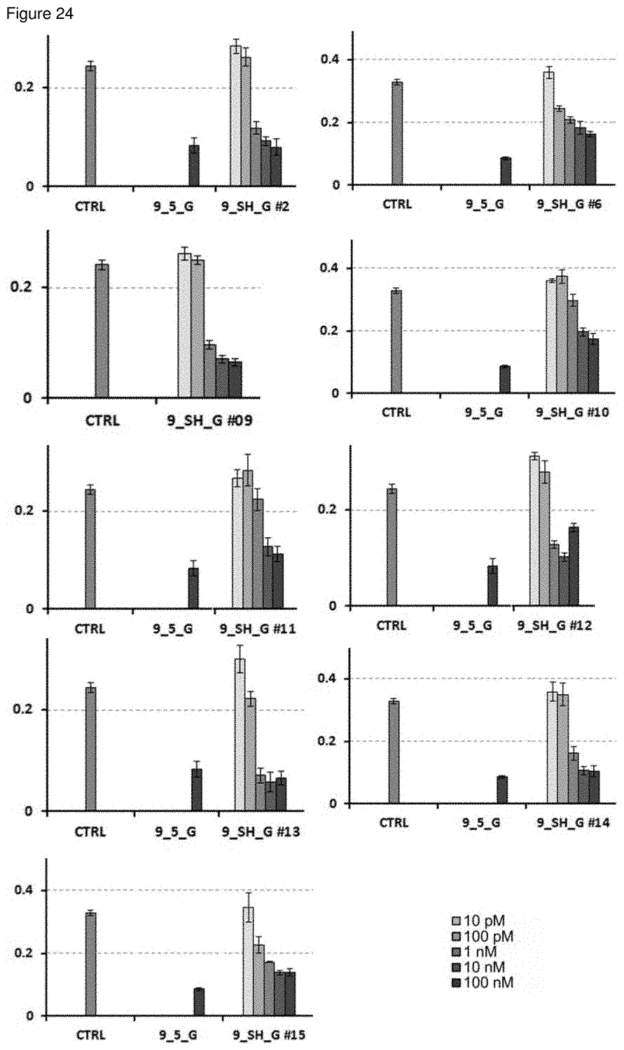

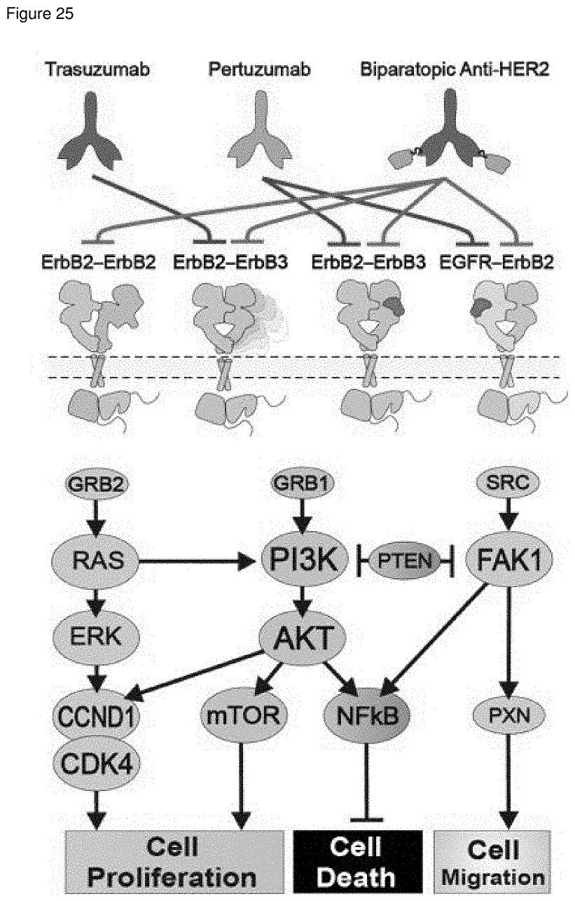

Additionally TUNEL assays as described in example 1 were performed with the above mentioned bispecific HER2 binding agents. As shown in FIG. 22, the percentage of TUNEL-positive cells is significantly higher for the tested bispecific agents than for trastuzumab. These results were verified by Western blot analysis, wherein the apoptosis was detected by the cleavage of Poly ADP Ribose Polymerase (FIG. 23). The Western blot analysis was performed as described in example 1.

In summary, it could be shown that the bispecific HER2 binding agents comprising one or two antibody fragments are able to trigger apoptosis of the targeted cell much better than trastuzumab.

### Example 6: Bispecific HER2 Bindings Agent Comprising Two DARPins Connected by a Shared Helix

The principle of the bispecific constructs, namely that an HER2_I and an HER2_IV binder are fused in order to bring the respective domains of two different HER2-molecules into proximity, does in principle work with flexible linkers of different lengths. As an alternative to this, DARPin constructs have been created in which the two DARPins have been fused rigidly in different angles and tested in cell viability assay as described in example 1. All 9 tested constructs 9.29_SH_G3 #2 (SEQ ID 102), 9.29_SH_G3 #6 (SEQ ID 103), 9.29_SH_G3 #9 (SEQ ID 104), 9.29_SH_G3 #10 (SEQ ID 105), 9.29_SH_G3 #11 (SEQ ID 106), 9.29_SH_G3 #12 (SEQ ID 107), 9.29_SH_G3 #13 (SEQ ID 108), 9.29_SH_G3 #14 (SEQ ID 109), and 9.29_SH_G3 #15 (SEQ ID 110) have strong anti-proliferative activity in cell viability assays with HER2-dependent cancer cells (BT474), however to varying degrees.

Without wishing to be bound by theory, it is supposed that the target (HER2) can orient in various orientations over the membrane insertion point. Still, in all different orientations, the two transmembrane helices of the bound receptors will be kept at a distance sufficient to inactivate the kinase activity. This blueprint allows some flexibility in the epitopes bound on HER2_I and HER2_IV and in the orientation with which these epitopes are bound.

### Example 7: Biparatopic Anti-HER2 Binding Agents

The inventors have developed another class of HER2 inhibitors, biparatopic anti-HER2 binding agents based on designed ankyrin repeat proteins (DARPins; e.g. 6L1G, 9L1H), which block all signaling-active interactions of HER2 receptor with itself (active homodimers) and with other receptor tyrosine kinases (heterodimers) (Jost et al., 2013; Tamaskovic et al., 2016). These pan-ErbB inhibitors block p-HER2 and p-HER3 to a similar extent as a combination of a small molecule kinase inhibitor against HER2 (such as ARRY-380) in combination with trastuzumab treatment. Furthermore, the biparatopic anti-HER2 DARPin agents robustly induced apoptosis in HER2-amplified breast cancer cell lines with a PI3K WT background (Tamaskovic et al., 2016).

Based on the same construction principles, the inventors have generated also biparatopic IgG derivatives. In contrast to other available biparatopic HER2-targeting antibodies, e.g. the antibody-drug conjugate (ADC) from Medimmune MEDI4276 (Li et al., 2016), these IgGs show very strong anti-tumor activity as “naked” binding proteins, i.e., without attached drug (Kast et al., in preparation). Thus, these novel biparatopic anti-HER2 IgGs combine the mechanisms of action of trastuzumab plus pertuzumab plus the action of small molecule kinases inhibitors against HER2 in one single molecule. In addition, potential off-target effects of the biparatopic anti-HER2 IgGs are expected to remain far below those of ADC fusions, such as T-DM1 or MEDI4276, as they can only act on HER2-addicted cells, while ADCs can via their toxin act in many healthy tissue. This opens up the therapeutic windows for new combination therapies. Furthermore, pan-ErbB inhibition by polymerization of HER2 receptors may passively block compensatory activation of other receptor tyrosine kinases (RTKs). The biparatopic anti-HER2 binding agents interfere with the free lateral movement of HER2 receptors on the cell surface of HER2-amplified cancer, yet without inducing signaling competent complexes, which may block the activation of other RTKs. Consequently, biparatopic anti-HER2 binding agents may show strong synergies with small molecule inhibitors, which tend to induce expression of compensatory RTKs that eventually drives escape from therapy. Therefore, biparatopic anti-HER2 IgGs bear a very high potential to elicit strong anti-tumor synergies in combination with small-molecule inhibitors on a broad panel of HER2-amplified cancers. The potential for synergies with small-molecule inhibitors is superior to current single-specificity antibodies or antibody combinations.

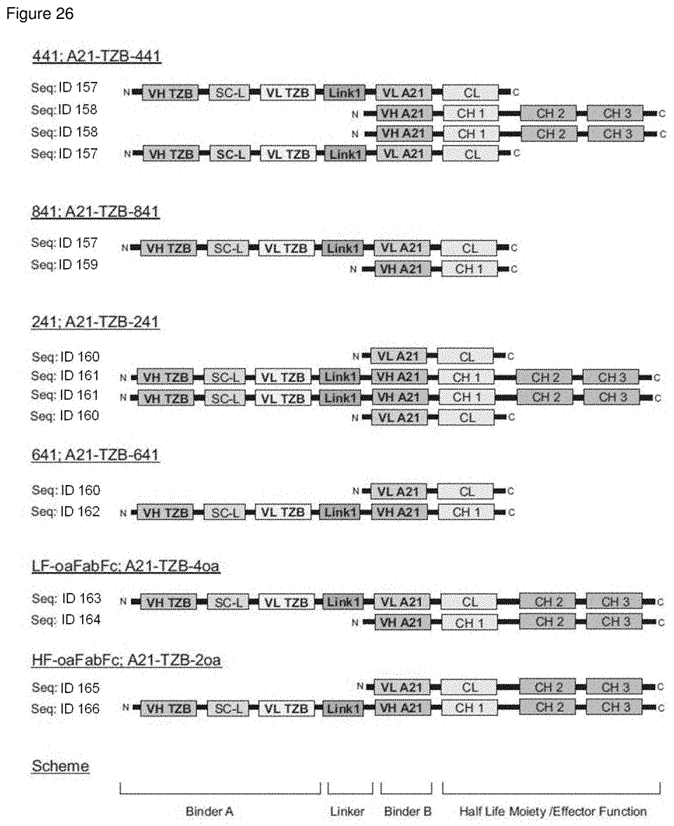

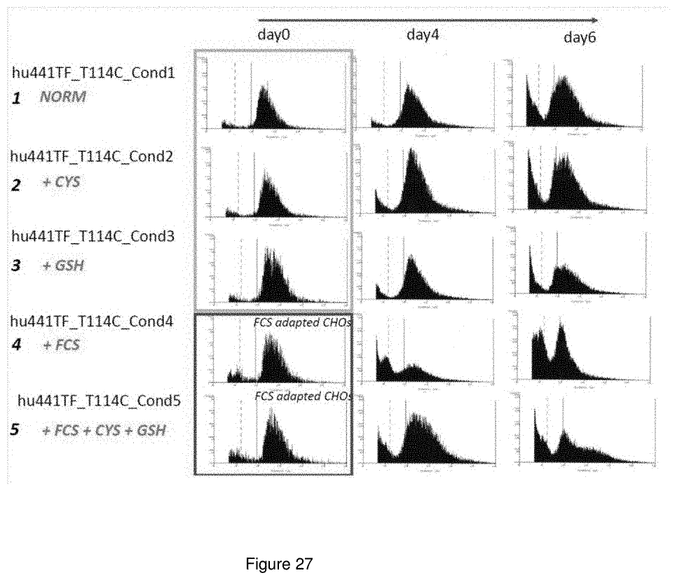

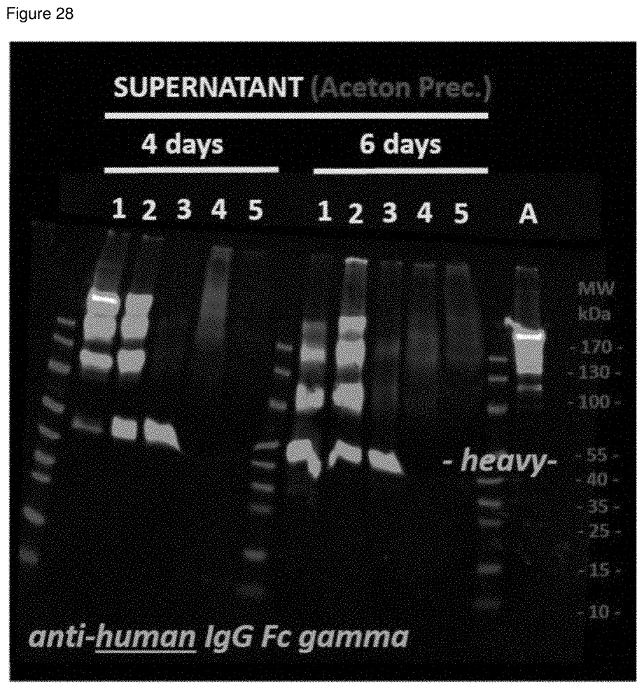

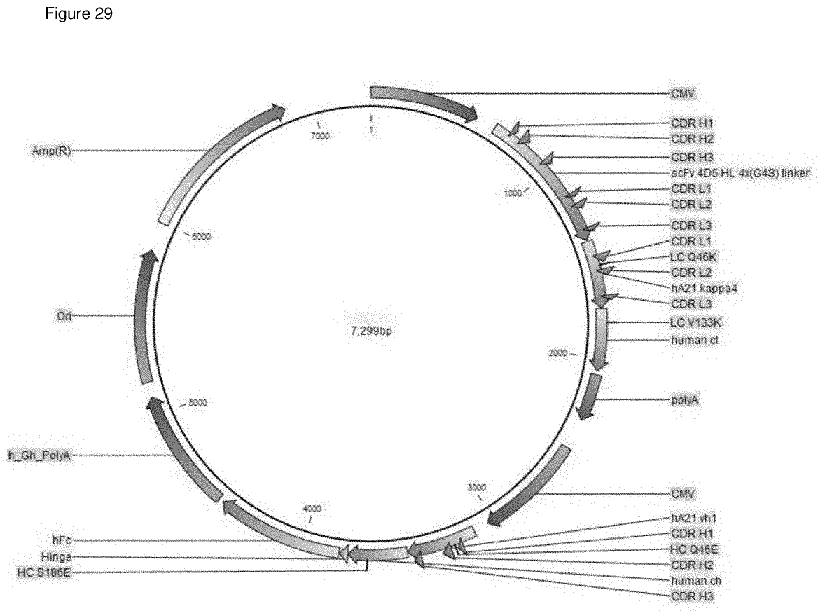

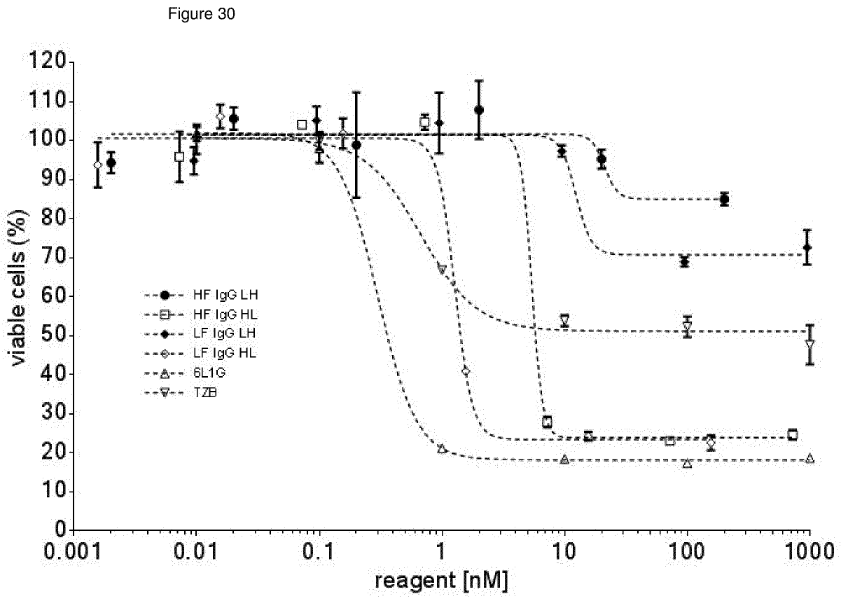

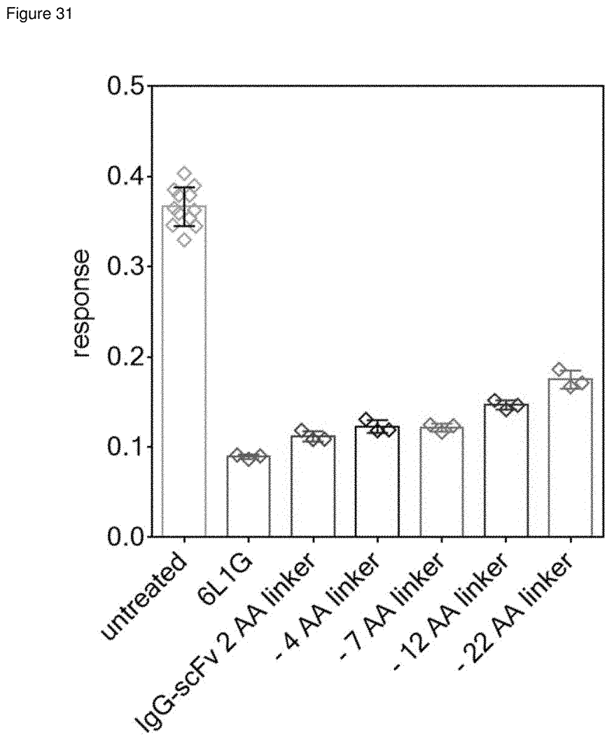

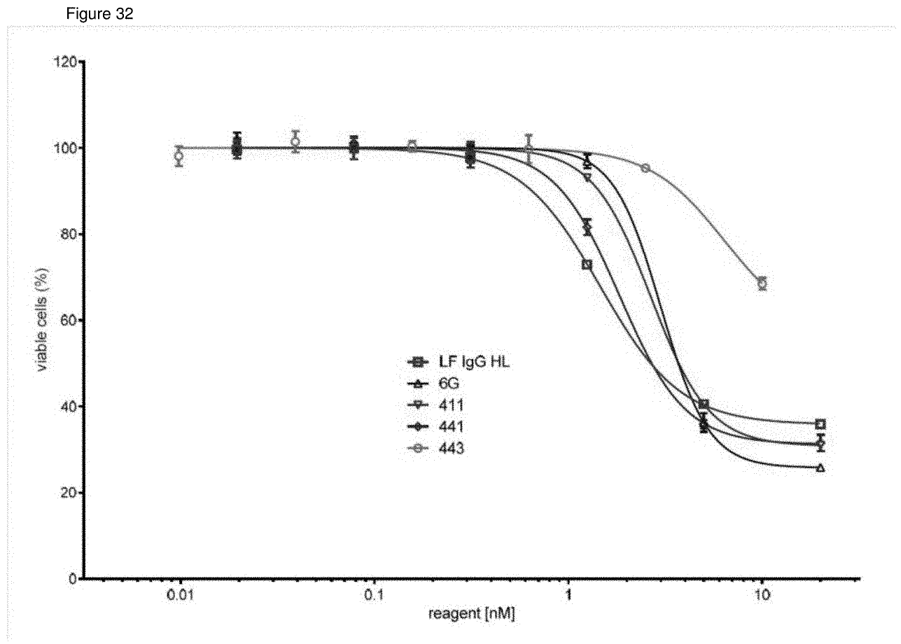

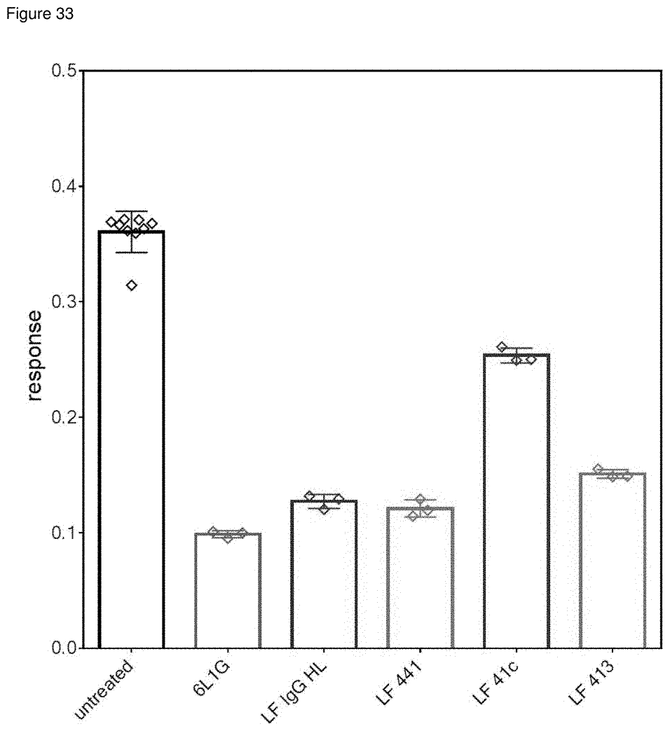

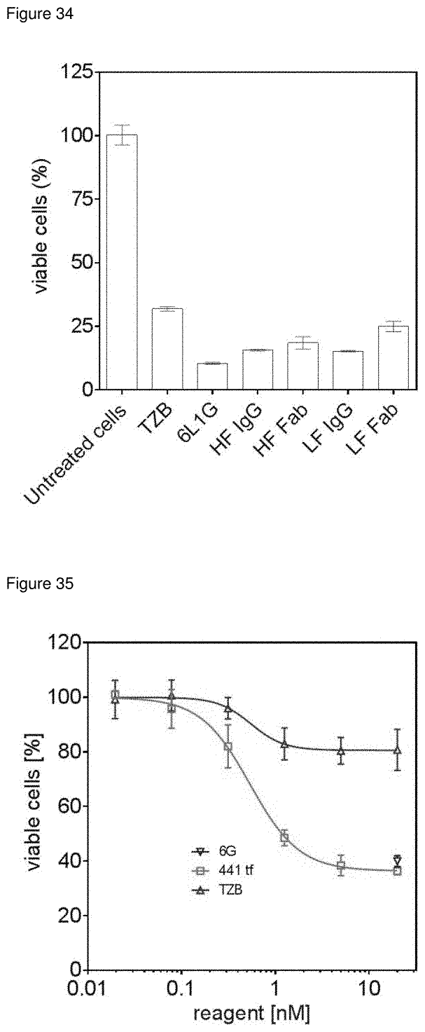

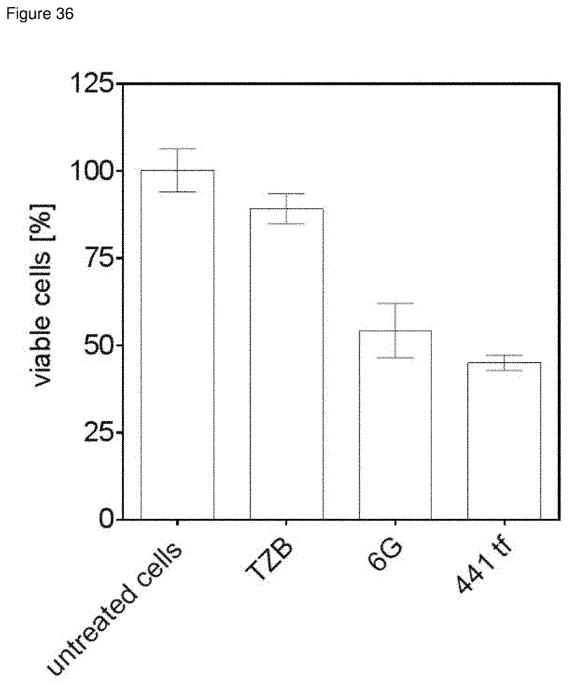

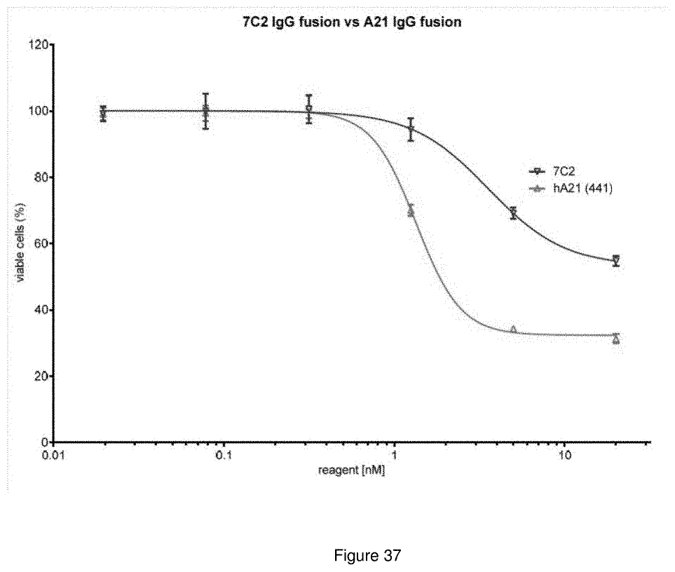

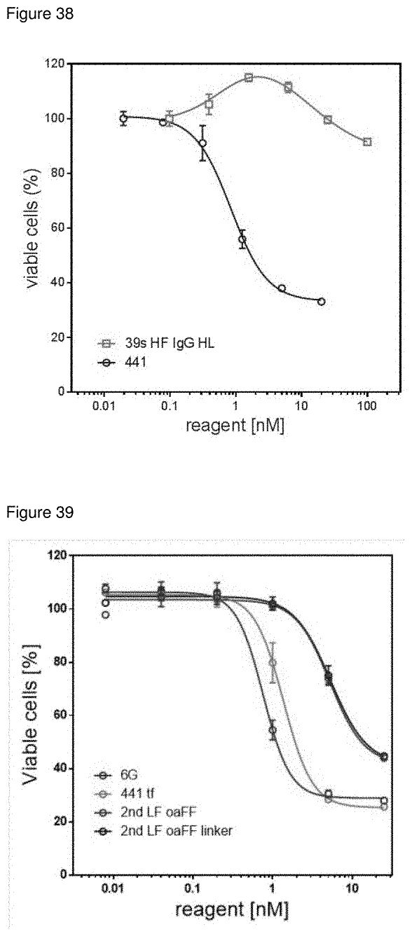

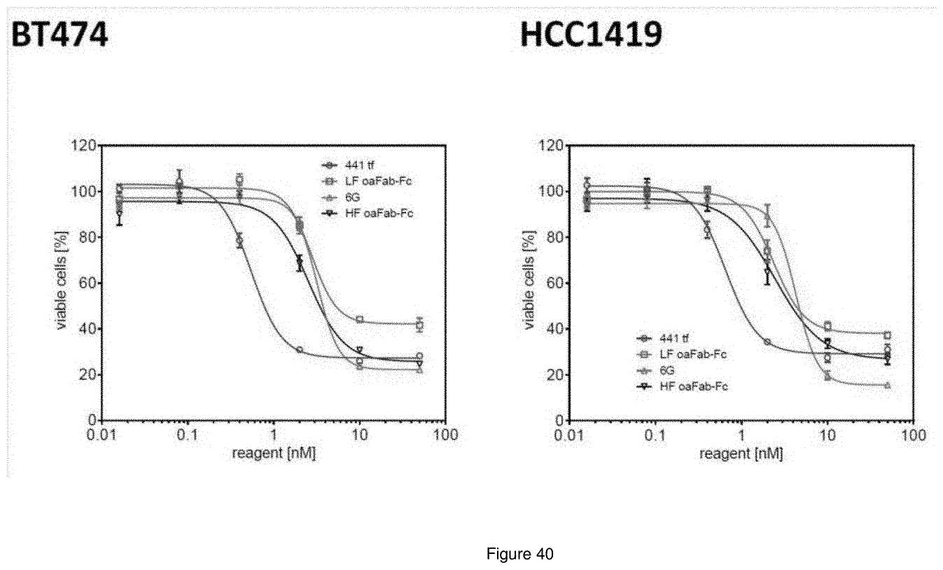

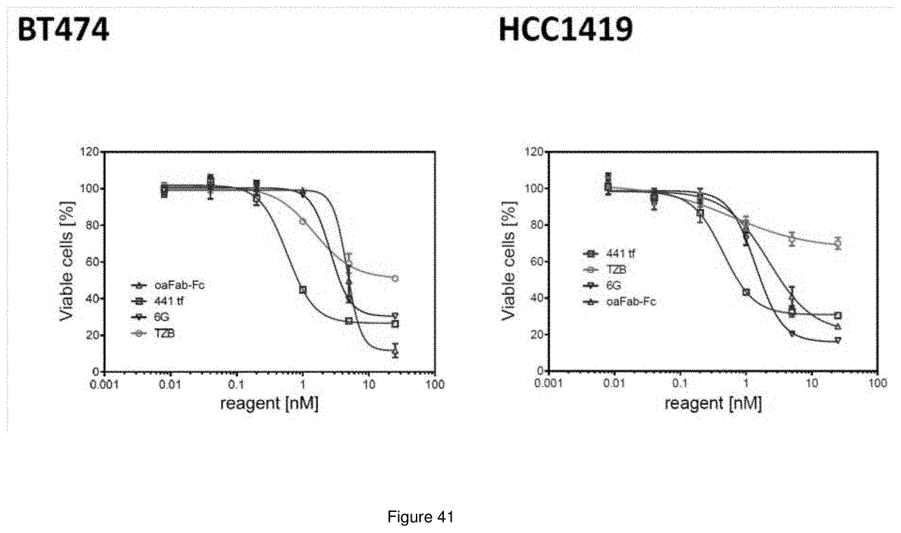

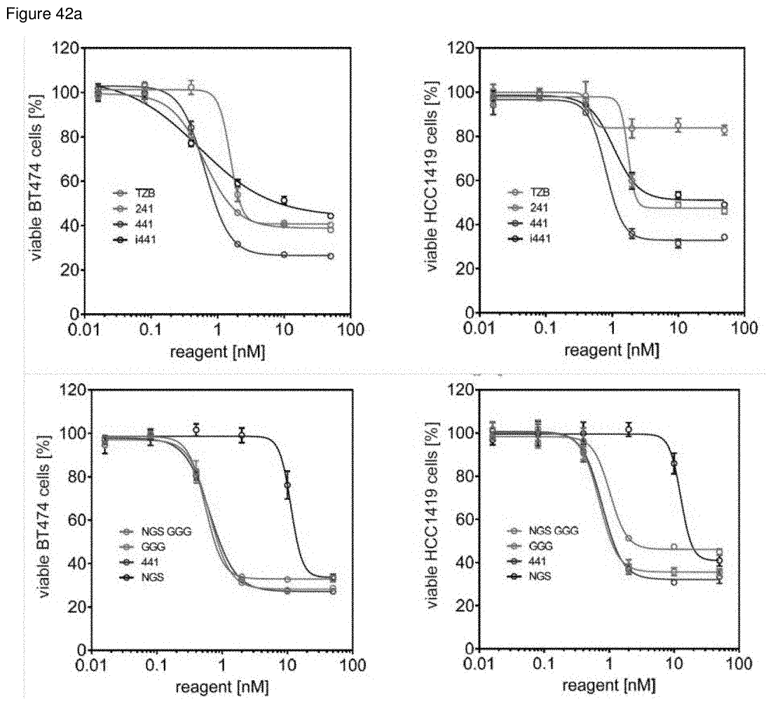

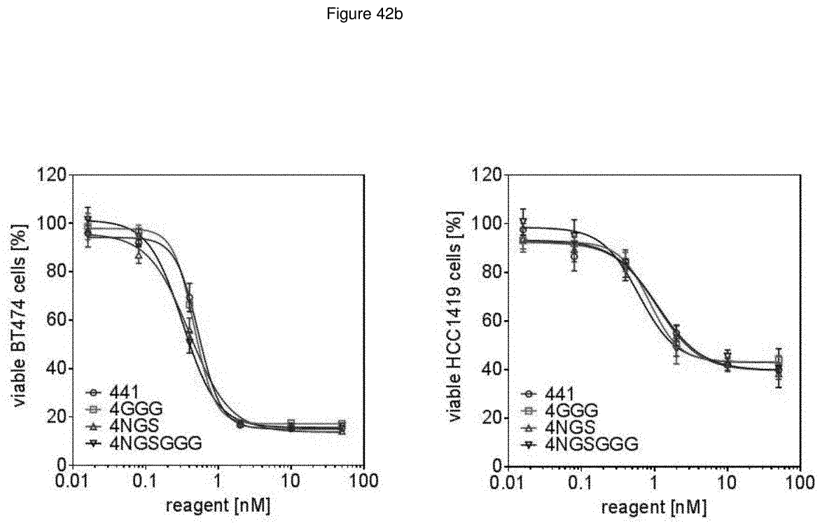

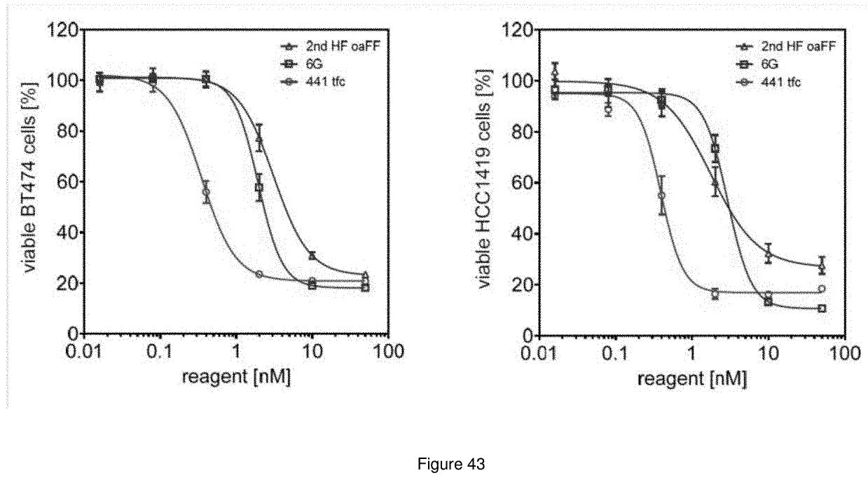

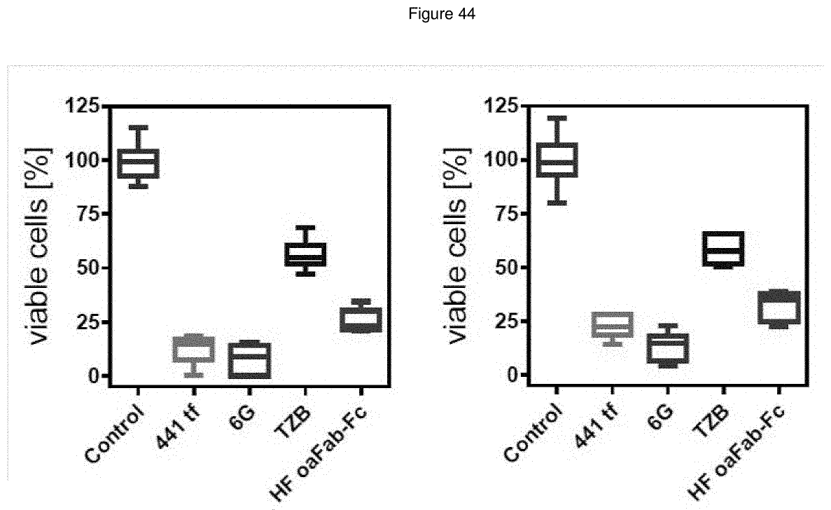

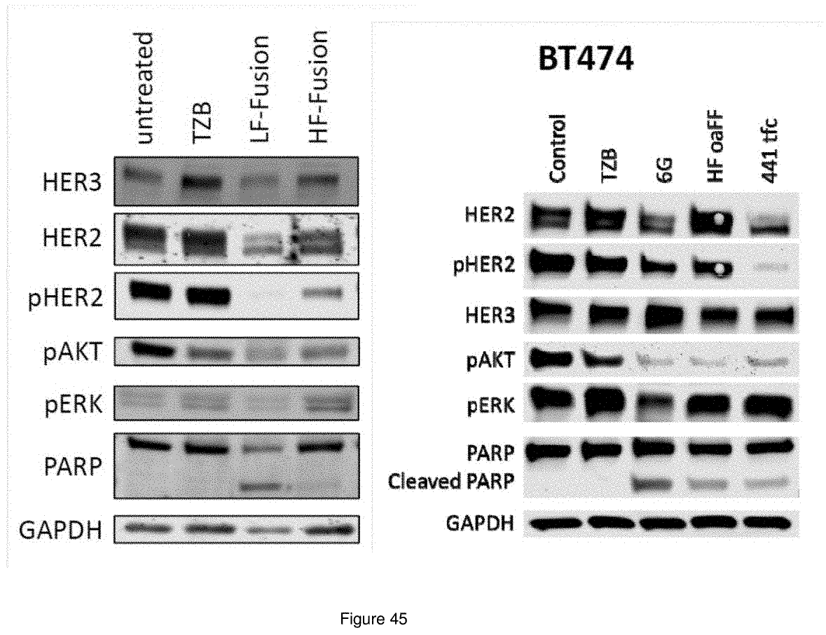

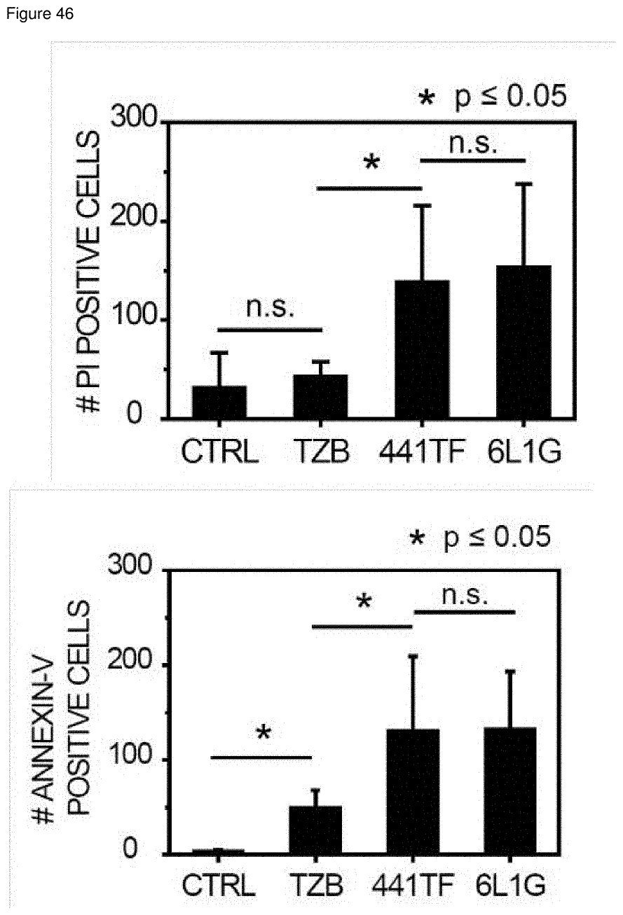

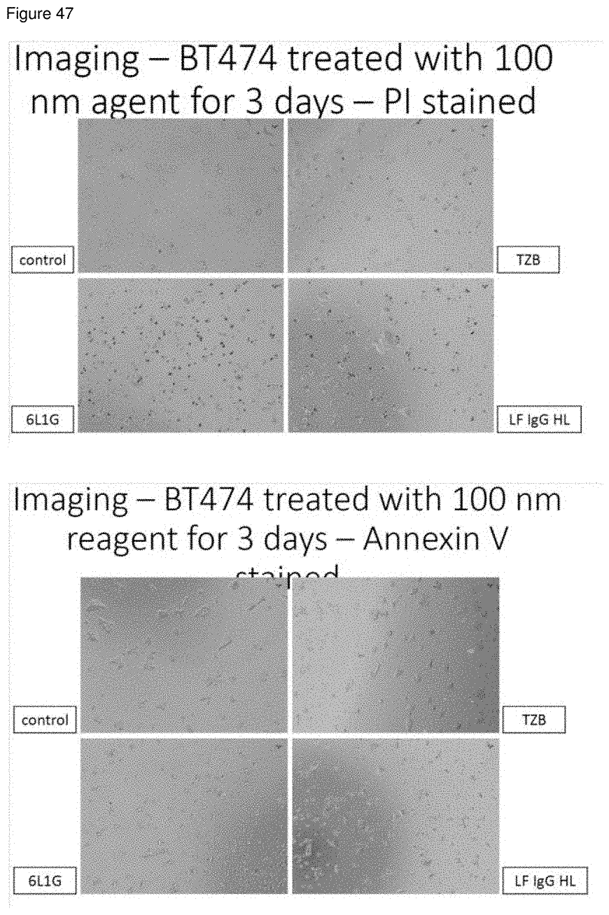

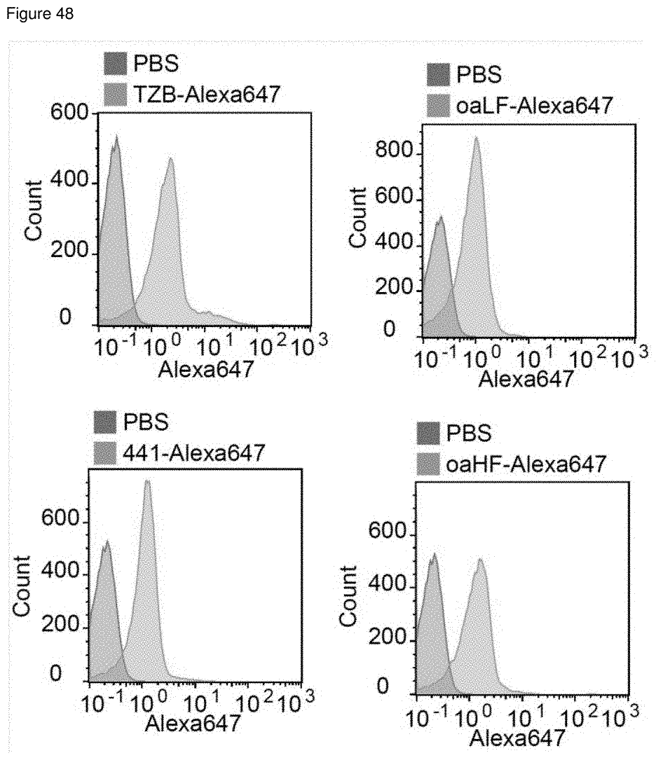

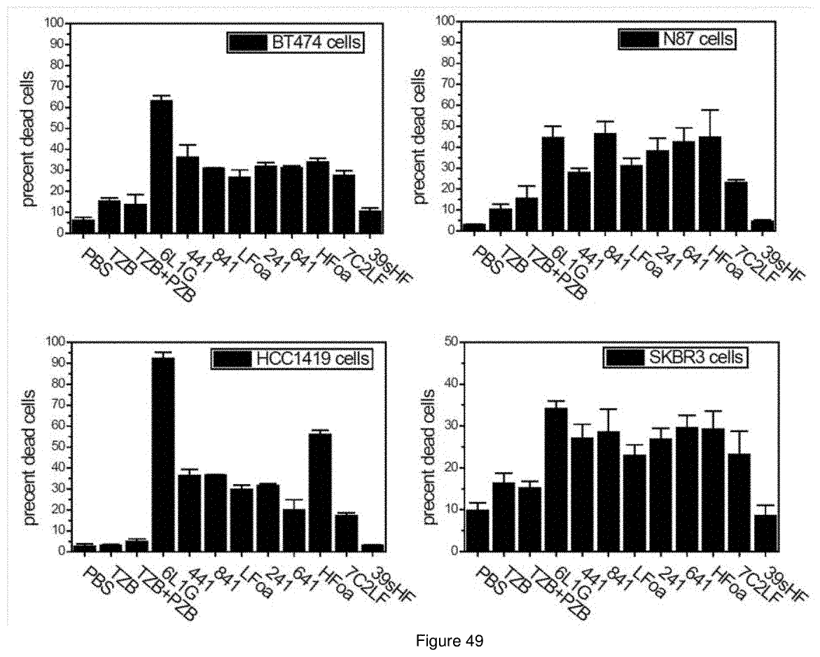

Illustrative schemes of preferred biparatopic IgG constructs are shown in FIG. 26 Data regarding preparation, and biological activity of the biparatopic IgG constructs are shown in FIGS. 27 to 50.

Protocol for Transient Production of Biparatopic IgGs in CHOs cells

PEI transfection of CHO-S cells in roller or shaker bottles

Materials

Polyethylenimine, Linear (MW 25,000) PolySciences Inc. (Stock of 1 mg/ml)

Dissolve by lowering pH to around 3 by use of 1 N HCl

Titrate back to pH of 7 by addition of 1 N NaOH

Sterilize by filtration (0.22 μm)

Freeze aliquots −20° C.

Media without antibiotics and antifungal agents

DNA endotoxin free, high quality

Day −1

Split cells to around 2.0*106 per ml in fresh media

Day 0

Per ml use 3 μg of PEI and 1.25 μg of DNA.

Split cells to 4.0*106 per ml in 200-250 ml of fresh media (TPP600, max 300 ml)

Add DNA and PEI sequentially and swirl in between

Incubate for desired days at 31° C. or 37° C.

On day 5-12 harvest cells by centrifugation (1200 g/4° C./30 min)

Filter SN through 0.22 μm and adjust pH according to next purification step

For estimation of transfection efficiency, use 2% GFP plasmid co-transfection. Analyse resulting transfection efficiency via flow cytometry of 1 ml washed cell suspension

Purification of Biparatopic IgGs from CHOgro Medium after Expression

Different biparatopic anti-HER2 IgG-scFv constructs could successfully be purified with a protein A purification procedure. 10×HEPES buffer pH 7.5 was added to expression medium to adjust the pH to protein A binding conditions. Supernatants were filtered through a 0.22 μm PVDF membrane prior loading to protein A columns (e.g. HiTrap rProtein A FF (GE Healthcare)). Afterwards, proteins were eluted with citric acid pH 2.5 to 2.8 and fractions of interest were subsequently neutralized by addition of 1 M Tris pH=8.

Protein A Purification of Fab-scFv Fragments

Also different biparatopic Fab-scFv fragments could successfully be purified with a protein A purification procedure of. 10×HEPES buffer pH 7.5 was added to the expression medium to adjust the pH to protein A binding conditions. Supernatants were filtered through a 0.22 μm PVDF membrane prior loading to protein A columns (e.g. HiTrap rProtein A FF (GE Healthcare)). Afterwards, proteins were eluted with citric acid pH 2.5 to 2.8 and fractions of interest were subsequently neutralized by addition of 1 M Tris pH=8.

Prep. SEC Purification of Fab-scFv

Fab-scFv fragments were purified to higher purity by preparative size exclusion chromatography on Superdex200 columns (GE Healthcare). The peak at around 14.5 ml corresponds to the desired monovalent full length product.

Purification of Biparatopic Anti-HER2 IgGs by Ion Exchange Chromatography

Different biparatopic anti-HER2 IgGs constructs could be effectively separated by either anion or cation exchange chromatography using Mono-Q ore Mono-S column materials. Resource Q or Resources S columns from GE Healthcare were used. BIS-TRIS buffer pH 6.75 was used for cation exchange. TRIS buffer pH 8 was used for anion exchange chromatography. Elution was performed with the same buffer containing 1 M NaCl.

Purification of Biparatopic Anti-HER2 IgGs by Size Exclusion Chromatography

Different biparatopic anti-HER2 IgG constructs could also be effectively separated by molecular size. Superdex200 from GE Healthcare were used for purification.

Purifity of the biparatopic IgG constructs was confirmed by Analytical SEC, LS-SEC, PAGE and MS.

We claim:

1. A bispecific HER2-targeting polypeptide comprising:
a first polypeptide ligand that binds to HER2 extracellular domain 1;
a second polypeptide ligand that binds to HER2 extracellular domain 4; and
a linker covalently attaching said first polypeptide ligand to said second polypeptide ligand, wherein said linker consists of 5 to 25 amino acids, and is composed of at least 50% glycine, alanine, proline, threonine, and/or serine residues;
wherein
I) the second polypeptide ligand is an antibody targeting HER2 domain 4, and the first polypeptide ligand is a polypeptide targeting HER2 domain 1 selected from an immunoglobulin variable domain, Fab fragment, scFv Fragment and an ankyrin based polypeptide, wherein said polypeptide is connected to
i) the N-terminus of a heavy chain of said antibody,
ii) the C-terminus of a heavy chain of said antibody,
iii) the N-terminus of a light chain of said antibody, or
iv) the C-terminus of a light chain of said antibody,
wherein said antibody targeting HER2 domain 4 comprises at least one CDR1 sequence selected from SEQ ID NO 116 and SEQ ID NO 119, at least one CDR2 sequence selected from SEQ ID NO 117 and 120, and at least one CDR3 sequence selected from SEQ ID NO 118, and SEQ ID NO 121, and
wherein said polypeptide targeting HER2 domain 1 is at least one sequence selected from the group consisting of SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 61, SEQ ID NO 62, SEQ ID NO 63, SEQ ID NO 64, SEQ ID NO 65, SEQ ID NO 66, SEQ ID NO 93, SEQ ID NO 122, SEQ ID NO 123, SEQ ID NO 124, SEQ ID NO 125, SEQ ID NO 126, SEQ ID NO 127, SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 143, SEQ ID NO 144, SEQ ID NO 145, SEQ ID NO 146, SEQ ID NO 147, SEQ ID NO 148, SEQ ID NO 149, SEQ ID NO 150, and SEQ ID NO 151;
or
II) the first polypeptide ligand is an antibody targeting HER2 domain 1, and the second polypeptide ligand is a polypeptide targeting HER2 domain 4 selected from an immunoglobulin variable domain, Fab fragment, scFv Fragment and an ankyrin based polypeptide, wherein said polypeptide targeting HER2 domain 4 is connected to
i) the N-terminus of a heavy chain of said antibody,
ii) the C-terminus of a heavy chain of said antibody,
iii) the N-terminus of a light chain of said antibody or
iv) the C-terminus of a light chain of said antibody,
wherein said antibody targeting HER2 domain 1 comprises at least one CDR1 sequence selected from SEQ ID NO 122 and SEQ ID NO 125, at least one CDR2 sequence selected from SEQ ID NO 123 and SEQ ID NO 126, and at least one CDR3 sequence selected from SEQ ID NO 124 and SEQ ID NO 127, and
wherein said polypeptide targeting HER2 domain 4 is at least one sequence selected from the group consisting of SEQ ID NO 25, SEQ ID NO 26, SEQ ID NO 27, SEQ ID NO 28, SEQ ID NO 29, SEQ ID NO 67, SEQ ID NO 68, SEQ ID NO 69, SEQ ID NO 92, SEQ ID NO 116, SEQ ID NO 117, SEQ ID NO 118, SEQ ID NO 119, SEQ ID NO 120, SEQ ID NO 121, SEQ ID NO 128, SEQ ID NO 129, SEQ ID NO 130, SEQ ID NO 131, SEQ ID NO 132, and SEQ ID NO 133.

2. The bispecific HER-targeting polypeptide of claim 1, wherein
the antibody targeting HER2 domain 4 comprises at least one sequence selected from one of SEQ ID NO 128, SEQ ID NO 129, SEQ ID NO 130, SEQ ID NO 131, SEQ ID NO 132, and SEQ ID NO 133, or
the antibody targeting HER2 domain 1 comprises at least one sequence selected from one of SEQ ID NO 134, SEQ ID NO 135, SEQ ID NO 136, SEQ ID NO 137, SEQ ID NO 138, SEQ ID NO 139, SEQ ID NO 140, SEQ ID NO 141, SEQ ID NO 142, SEQ ID NO 144, SEQ ID NO 145, SEQ ID NO 146, SEQ ID NO 147, SEQ ID NO 148, SEQ ID NO 149, SEQ ID NO 150, and SEQ ID NO 151.

3. The bispecific HER2-targeting polypeptide of claim 1, wherein the antibody comprises a Fc domain comprising a sequence selected from SEQ ID NO 152, SEQ ID NO 153, SEQ ID NO 154, SEQ ID NO 155, and SEQ ID NO 156.

4. The bispecific HER2-targeting polypeptide of claim 1, wherein the first polypeptide ligand and the second polypeptide ligand are attached to each other by an oligopeptide linker, and the first polypeptide ligand, second polypeptide ligand and linker form one continuous polypeptide chain.

5. The bispecific HER2-targeting polypeptide of claim 4, wherein the first polypeptide sequence is located at the N-terminus of the continuous polypeptide chain, the second polypeptide sequence is located at the C-terminus of the continuous polypeptide chain, and the linker is located between the first and the second polypeptide ligand.

6. The bispecific HER2-targeting polypeptide according to claim 1, wherein the linker is a polyglycine/serine linker comprising one or a multiple of an amino acid sequence set forth herein as SEQ ID NO 51 ((GGGGS)ₙ), wherein n is 1, 2, 3, 4 or 5.

7. The bispecific HER2-targeting polypeptide of claim 1, wherein the linker comprises a sequence selected from one of SEQ ID NO 51, SEQ ID NO 52, SEQ ID NO 53, SEQ ID NO 54, SEQ ID NO 111, SEQ ID NO 167, SEQ ID NO 168, SEQ ID NO 169, SEQ ID NO 170, SEQ ID NO 171, SEQ ID NO 172, SEQ ID NO 173, SEQ ID NO 174, SEQ ID NO 175, SEQ ID NO 176, SEQ ID NO 177, SEQ ID NO 178, SEQ ID NO 179, SEQ ID NO 180, SEQ ID NO 181, SEQ ID NO 182, SEQ ID NO 183, SEQ ID NO 184, and SEQ ID NO 185.

8. The bispecific HER2-targeting polypeptide of claim 1, wherein the linker comprises a sequence selected from one of SEQ ID NO 167, SEQ ID NO 168, SEQ ID NO 169, SEQ ID NO 170, SEQ ID NO 171, SEQ ID NO 172, SEQ ID NO 173, SEQ ID NO 174, SEQ ID NO 175, SEQ ID NO 176, SEQ ID NO 177, SEQ ID NO 178, SEQ ID NO 179, SEQ ID NO 180, SEQ ID NO 181, SEQ ID NO 182, SEQ ID NO 183, SEQ ID NO 184, SEQ ID NO 185, and SEQ ID NO 186.

9. A bispecific HER2-targeting peptide molecule comprising at least one an amino acid sequence selected from any one of SEQ ID NO 157, SEQ ID NO 158, SEQ ID NO 159, SEQ ID NO 160, SEQ ID NO 161, SEQ ID NO 162, SEQ ID NO 163, SEQ ID NO 164, SEQ ID NO 165, SEQ ID NO 166, and SEQ ID NO 187.

10. An isolated nucleic acid molecule encoding the bispecific HER2-targeting polypeptide of claim 1.

11. A method for treating a HER2 positive cancer, comprising administering to a patient in need thereof the bispecific HER2 polypeptide of claim 1.
